SEQUENCE LISTING (68 sequences)

SEQ ID NO: 1 - protein, 227 aa, Homo sapiens
MQDEDGYITLNIKTRKPALISVGSASSSWWRVMALILLILCVGMVVGLVALGIWSVMQRNYLQDENENRTGTLQQLAKRCQYVVKQSELKGTFKGHKCSPCDTNWRYYGDSCYGFFRHNLTWEESKQYCTDMNATLLKIDNRNIVEYIKARTHLIRWVGLSRQKSNEVWKWEDGSVISENMFEFLEDGKGNMNCAYFHNGKMHPTCENKHYLMCERKAGMTKVDQLP

SEQ ID NO: 2 - protein, 170 aa, Homo sapiens
QRNYLQDENENRTGTLQQLAKRCQYVVKQSELKGTFKGHKCSPCDTNWRYYGDSCYGFFRHNLTWEESKQYCTDMNATLLKIDNRNIVEYIKARTHLIRWVGLSRQKSNEVWKWEDGSVISENMFEFLEDGKGNMNCAYFHNGKMHPTCENKHYLMCERKAGMTKVDQLP

SEQ ID NO: 3 - protein, 180 aa, Artificial sequence
  His-tagged CLEC-2
HHHHHHHHHHQRNYLQDENENRTGTLQQLAKRCQYVVKQSELKGTFKGHKCSPCDTNWRYYGDSCYGFFRHNLTWEESKQYCTDMNATLLKIDNRNIVEYIKARTHLIRWVGLSRQKSNEVWKWEDGSVISENMFEFLEDGKGNMNCAYFHNGKMHPTCENKHYLMCERKAGMTKVDQLP

SEQ ID NO: 4 - DNA, 82 bp, Artificial sequence
  Synthetic oligomer
tctcggatcctcagcgagtcgtctgnnnnnnnnnnnnnnnnnnnnnnnnnnnnnnnnnnnnnnnnccgcatcgtcctcccta

SEQ ID NO: 5 - DNA, 40 bp, Artificial sequence
  Synthetic oligomer
actgggctcatattcccgcgcatcttactacctcggttat

SEQ ID NO: 6 - RNA, 40 nt, Artificial sequence
  Synthetic oligomer
acugggcucauauucccgcgcaucuuacuaccucgguuau

SEQ ID NO: 7 - DNA, 80 bp, Artificial sequence
  Synthetic oligomer
gggaggacgatgcggactgggctcatattcccgcgcatcttactacctcggttatcagacgactcgctgaggatccgaga

SEQ ID NO: 8 - RNA, 80 nt, Artificial sequence
  Synthetic oligomer
gggaggacgaugcggacugggcucauauucccgcgcaucuuacuaccucgguuaucagacgacucgcugaggauccgaga

SEQ ID NO: 9 - RNA, 80 nt, Artificial sequence; identical to SEQ ID NO: 8
  Synthetic oligonucleotide

SEQ ID NO: 10 - RNA, 80 nt, Artificial sequence; identical to SEQ ID NO: 8
  Synthetic oligomer

SEQ ID NO: 11 - RNA, 55 nt, Artificial sequence
  Synthetic oligomer
gggaggacgaugcggacugggcucauauucccgcgcaucuuacuaccucgguuau

SEQ ID NO: 12 - RNA, 49 nt, Artificial sequence
  Synthetic oligomer
gggaggacgaugcggacugggcucauauucccgcgcaucuuacuaccuc

SEQ ID NO: 13 - RNA, 45 nt, Artificial sequence
  Synthetic oligomer
gggaggacgaugcggacugggcucauauucccgcgcaucuuacua

SEQ ID NO: 14 - RNA, 39 nt, Artificial sequence
  Synthetic oligomer
gggaggacgaugcggacugggcucauauucccgcgcauc

SEQ ID NO: 15 - RNA, 33 nt, Artificial sequence
  Synthetic oligomer
gggaggacgaugcggacugggcucauauucccg

SEQ ID NO: 16 - RNA, 35 nt, Artificial sequence
  Synthetic oligomer
gggagaugcggacugggcucauauucccgcgcauc

SEQ ID NO: 17 - RNA, 37 nt, Artificial sequence
  Synthetic oligomer
gggagaugcggacugggcucauauucccgcgcaucuc

SEQ ID NO: 18 - RNA, 39 nt, Artificial sequence
  Synthetic oligomer
gggagaugcggacugggcucauauucccgcgcaucuccc

SEQ ID NO: 19 - RNA, 33 nt, Artificial sequence
  Synthetic oligomer
gggagagcggacugggcucauauucccgcgcuc

SEQ ID NO: 20 - RNA, 33 nt, Artificial sequence
  Synthetic oligomer
gggagaucggacugggcucauauucccgcgauc

SEQ ID NO: 21 - RNA, 33 nt, Artificial sequence
  Synthetic oligomer
gggagaugggacugggcucauauucccgccauc

SEQ ID NO: 22 - RNA, 33 nt, Artificial sequence
  Synthetic oligomer
gggaugcggacugggcucauauucccgcgcauc

SEQ ID NO: 23 - RNA, 33 nt, Artificial sequence
  Synthetic oligomer
gggaugcggaccgggcucauauucccgcgcauc

SEQ ID NO: 24 - RNA, 31 nt, Artificial sequence
  Synthetic oligomer
gaugcggacugggcucauauucccgcgcauc

SEQ ID NO: 25 - RNA, 35 nt, Artificial sequence
  Synthetic oligomer
gggagaugcgcucugggcucauauucccgcgcauc

SEQ ID NO: 26 - RNA, 35 nt, Artificial sequence
  Synthetic oligomer
gggagaugcggacuggggacauauucccgcgcauc

SEQ ID NO: 27 - RNA, 35 nt, Artificial sequence
  Synthetic oligomer
gggagaugcggacugggcuguuauucccgcgcauc

SEQ ID NO: 28 - RNA, 35 nt, Artificial sequence
  Synthetic oligomer
gggagaugcggacugggcucaauuucccgcgcauc

SEQ ID NO: 29 - RNA, 35 nt, Artificial sequence
  Synthetic oligomer
gggagaugcggacugggcucauaaacccgcgcauc

SEQ ID NO: 30 - RNA, 35 nt, Artificial sequence
  Synthetic oligomer
gggagaugcggagugggcucauauucccgcgcauc

SEQ ID NO: 31 - RNA, 35 nt, Artificial sequence
  Synthetic oligomer
gggagaugcgcacugggcucauauucccgcgcauc

SEQ ID NO: 32 - RNA, 31 nt, Artificial sequence
  Synthetic oligomer
gaugcgcacugggcucauauucccgcgcauc

SEQ ID NO: 33 - RNA, 35 nt, Artificial sequence
  Synthetic oligomer
gggagaugcggucugggcucauauucccgcgcauc

SEQ ID NO: 34 - RNA, 33 nt, Artificial sequence
  Synthetic oligomer
gggaugcggacuggggucauauucccgcgcauc

SEQ ID NO: 35 - RNA, 33 nt, Artificial sequence
  Synthetic oligomer
gggaugcggacugggcacauauucccgcgcauc

SEQ ID NO: 36 - RNA, 33 nt, Artificial sequence
  Synthetic oligomer
gggaugcggacugggcugauauucccgcgcauc

SEQ ID NO: 37 - RNA, 33 nt, Artificial sequence
  Synthetic oligomer
gggaugcggacugggcucuuauucccgcgcauc

SEQ ID NO: 38 - RNA, 33 nt, Artificial sequence
  Synthetic oligomer
gggaugcggacugggcucaaauucccgcgcauc

SEQ ID NO: 39 - RNA, 33 nt, Artificial sequence
  Synthetic oligomer
gggaugcggacugggcucauuuucccgcgcauc

SEQ ID NO: 40 - RNA, 33 nt, Artificial sequence
  Synthetic oligomer
gggaugcggacugggcucauaaucccgcgcauc

SEQ ID NO: 41 - RNA, 33 nt, Artificial sequence
  Synthetic oligomer
gggaugcggacugggcucauauacccgcgcauc

SEQ ID NO: 42 - RNA, 33 nt, Artificial sequence
  Synthetic oligomer
gggaugcggacuggguucauauucccgcgcauc

SEQ ID NO: 43 - RNA, 33 nt, Artificial sequence
  Synthetic oligomer
gggaugcggacugggcccauauucccgcgcauc

SEQ ID NO: 44 - RNA, 33 nt, Artificial sequence
  Synthetic oligomer
gggaugcggacugggcuuauauucccgcgcauc

SEQ ID NO: 45 - RNA, 33 nt, Artificial sequence
  Synthetic oligomer
gggaugcggacugggcucguauucccgcgcauc

SEQ ID NO: 46 - RNA, 33 nt, Artificial sequence
  Synthetic oligomer
gggaugcggacugggcucacauucccgcgcauc

SEQ ID NO: 47 - RNA, 33 nt, Artificial sequence
  Synthetic oligomer
gggaugcggacugggcucauguucccgcgcauc

SEQ ID NO: 48 - RNA, 33 nt, Artificial sequence
  Synthetic oligomer
gggaugcggacugggcucauacucccgcgcauc

SEQ ID NO: 49 - RNA, 33 nt, Artificial sequence
  Synthetic oligomer
gggaugcggacugggcucauauccccgcgcauc

SEQ ID NO: 50 - RNA, 33 nt, Artificial sequence
  Synthetic oligomer
gggaugcgaacugggcucauauucccgcgcauc

SEQ ID NO: 51 - RNA, 33 nt, Artificial sequence
  Synthetic oligomer
gggaugcggauugggcucauauucccgcgcauc

SEQ ID NO: 52 - RNA, 33 nt, Artificial sequence
  Synthetic oligomer
gggaugcggacugggcucaucuucccgcgcauc

SEQ ID NO: 53 - RNA, 32 nt, Artificial sequence
  Synthetic oligomer
gggaugcggacugggcucuauucccgcgcauc

SEQ ID NO: 54 - RNA, 32 nt, Artificial sequence
  Synthetic oligomer
gggaugcggacugggcucauuucccgcgcauc

SEQ ID NO: 55 - RNA, 39 nt, Artificial sequence
  Synthetic oligomer
gggaggacgaugcggucugggcucauauucccgcgcauc

SEQ ID NO: 56 - RNA, 39 nt, Artificial sequence
  Synthetic oligomer
gggaggacgaugcgggcugggcucauauucccgcgcauc

SEQ ID NO: 57 - RNA, 39 nt, Artificial sequence
  Synthetic oligomer
gggaggacgaugcggacugggcucagauucccgcgcauc

SEQ ID NO: 58 - RNA, 33 nt, Artificial sequence
  Synthetic oligomer
gggaugcguacugggcucauauucccgcgcauc

SEQ ID NO: 59 - RNA, 32 nt, Artificial sequence
  Synthetic oligomer
gggaugcggacugggcucaauucccgcgcauc

SEQ ID NO: 60 - RNA, 16 nt, Artificial sequence
  Synthetic oligomer
gaugcgcgggaauaug

SEQ ID NO: 61 - RNA, 16 nt, Artificial sequence
  Synthetic oligomer
ugcgcgggaauaugag

SEQ ID NO: 62 - RNA, 15 nt, Artificial sequence
  Synthetic oligomer
aauaugagcccaguc

SEQ ID NO: 63 - RNA, 16 nt, Artificial sequence
  Synthetic oligomer
uaugagcccaguccgc

SEQ ID NO: 64 - RNA, 18 nt, Artificial sequence
  Synthetic oligomer
augagcccaguccgcauc

SEQ ID NO: 65 - RNA, 13 nt, Artificial sequence
  Synthetic oligomer
cccaguccgcauc

SEQ ID NO: 66 - DNA, 74 bp, Artificial sequence
  Synthetic oligomer
gggggaattctaatacgactcactatagggaggacgatgcggtaatacgactcactatagggaggacgatgcgg

SEQ ID NO: 67 - DNA, 25 bp, Artificial sequence
  Synthetic oligomer
tctcggatcctcagcgagtcgtctg

SEQ ID NO: 68 - DNA, 32 bp, Artificial sequence
  Synthetic oligomer
gaugcggacugggcucauauucccgcgcaucn

(19) United States
(12) Patent Application Publication
(10) Pub. No.: US 2012/0095085 A1
(43) Pub. Date: Apr. 19, 2012

(54) NUCLEIC ACID MODULATORS OF CLEC-2

(76) Inventors: JULIANA M. LAYZER, Durham, NC (US); Sanjoy K. Mahanty, Chapel Hill, NC (US); Samuel C. Wolff, Durham, NC (US); Catherine C. Redick, Durham, NC (US); Christopher P. Rusconi, Durham, NC (US)

(21) Appl. No.: 13/274,189

(22) Filed: Oct. 14, 2011

Related U.S. Application Data
(60) Provisional application No. 61/393,191, filed on Oct. 14, 2010.

(51) Int. Cl. A61K 31/7088; A61P 7/02; C07H 21/04; C07H 21/02; G01N 21/84

(52) U.S. Cl. 514/44 R; 536/23.1; 436/63

(57) ABSTRACT

Provided are ligands which bind to and regulate the function of CLEC-2. Nucleic acid CLEC-2 ligands described herein are able to inhibit CLEC-2 mediated platelet aggregation and may also provide use in regulating CLEC-2-mediated processes such as thrombus formation, tumor metastasis, lymphangiogenesis, HIV dissemination, inflammatory response, cytokine production and phagocytosis. Also disclosed herein are modulator molecules which can reverse the activity of the CLEC-2 ligand both in vitro and in vivo and ex vivo.

### CROSS-REFERENCE TO RELATED APPLICATIONS

This application claims the benefit of priority of U.S. Provisional Application No. 61/393,191, filed on Oct. 14, 2010, which is incorporated herein by reference in its entirety.

### TECHNICAL FIELD

The present invention relates, in general, to a pharmacologic system comprising a nucleic acid ligand that binds to and regulates the activity of the protein CLEC-2. These nucleic acid ligands are also actively reversible using a modulator that inhibits the activity of the nucleic acid ligand to neutralize its pharmacologic effect and thereby restore CLEC-2 function. The invention further relates to compositions comprising the nucleic acid ligand and/or a modulator as well as methods of using these agents and compositions in treating CLEC-2 mediated disorders.

### BACKGROUND

A C-type lectin-like receptor 2 (CLEC-2) has recently been identified using a bioinformatics approach and has been recognized as a platelet receptor (O'Callaghan, C. A., Current Opinion of Pharmacology, 2009; 9:90-95.). CLEC-2 is a type 2 transmembrane protein with an extracellular C-type lectin-like domain and a short cytoplasmic domain containing a single hemITAM (immunoreceptor tyrosine-based activation motif) YXXL motif. Phosphorylation of tyrosine 7 in the cytoplasmic YXXL motif is triggered by ligand binding upon activation of Src kinases, which in turn phosphorylates a series of downstream signaling proteins, including PLCγ2. There are two known naturally occurring proteins capable of interacting with CLEC-2 and mediating its physiologic signal transduction functions: the endogenous protein podoplanin and the snake venom protein rhodocytin, both of which trigger platelet activation, including potent stimulation of both aggregation and secretion. Inoue-Suzuki et. al., in collaboration with Kato reported that the profile of podoplanin-induced platelet aggregation is quite similar to that of rhodocytin-induced platelet aggregation and identified CLEC-2 as a receptor for podoplanin (Inoue-Suzuki, K. O. et. al., J. Thromb. Haemost. 2011; 9 (suppl 1): 44-55). They have also shown that rhodocytin-induced platelet aggregation is highly dependent on TxA2 generation and actin polymerization compared with platelet aggregation induced by CRP (Inoue, K. et. al. Biochem. Biophys. Res. Commun. 1999; 256: 114-120). Both rhodocytin and podoplanin interact directly with CLEC-2 at micromolar affinities (Kato, Y. et. al., Cancer Sci, 2008; 99:54-61; Ozaki, Y, et. al., J. Throm. Hemost, 2008; 7:191-194). CLEC-2 has been shown to bind to tumor cells via podoplanin, and results from mice studies indicate that CLEC-2 signaling may promote tumor metastasis (Watson, A. A., et al., Biochemistry, 2009; 48:10988-10996).

CLEC-2 has also been implicated in normal hemostasis and thrombosis in vivo (Spalton, J. C. J. Throm, Hemost, 2009; 7:1192-1199; Watson, A. A., et al., Biochemistry, 2009; 48:10988-10996) in propagating thrombus formation. Exposure of the subendothelial extracellular matrix proteins upon vessel wall damage triggers platelet activation and aggregation. Recently it has been shown in mice that anti-CLEC-2 antibody treatment reduces the protein from circulating platelets for several days. CLEC-2 deficient mice showed severe platelet aggregate formation defects ex vivo and in vivo (May, F., et al., Blood, 2009; 114:3464-3472). Thrombus formation under flow conditions ex vivo and in vivo is severely defective when mice are deficient of CLEC-2. CLEC-2 deficient mice exhibit a moderate increase in bleeding time compared to mice treated with agents that inhibit αIIbβ3 or those that inhibit GP1bα (May, F., et al., Blood, 2009; 114:3464-3472; Kunicki, T., Blood, 2009; 114:3364-3365).

CLEC-2 has also been found to mediate human immunodeficiency virus type 1 (HIV-1) attachment to platelets. The binding of CLEC-2 receptors on platelets to HIV-1 may allow HIV-1 to both avoid destruction by platelets and to use the platelets to facilitate its transmission (Chaipan, C. et. al., J. Virol. 2006; 80 (18): 8951-8960). The CLEC-2 receptor signaling has also been implicated in regulating the inflammatory response in myeloid dendritic cells (Mourao-Sa, D. et. al., 2011 Eur. J. Immun. 41:3040-3053).

Accordingly, a need exists for therapeutic agents and methods for treating CLEC-2 mediated disorders.

### BRIEF SUMMARY

Described herein are nucleic acid ligands which specifically inhibit CLEC-2, methods for identifying and/or characterizing these ligands, and treatments for use of these ligands.

In one aspect, a CLEC-2 ligand, or pharmaceutically acceptable salt thereof, is provided, wherein the ligand comprises an isolated nucleic acid sequence. In one embodiment, at least one nucleotide is a ribonucleotide. In another embodiment, at least one nucleotide is a 2′-fluoro-2′ deoxypyrimidine nucleotide. In still another embodiment, the isolated nucleic acid sequence of the CLEC-2 ligand comprises a mixture of ribonucleotides and 2′-fluoro-2′ deoxypyrimidine nucleotides.

In one embodiment, the CLEC-2 ligand comprises a secondary structure comprising at least one stem and at least one loop.

In one embodiment, the CLEC-2 ligand comprises, in a 5′ to 3′ direction: a first stem which is 5-10 basepairs (bp) in length; a first trinucleotide loops which comprises the sequence 5′-GNC-3′; a second stem which is 4 bp in length, wherein said second stem comprises a wobble pair at the base of the second stem; and a second loop comprising the nucleotide sequence 5′-YUYNNRYU-3′.

In one embodiment, the CLEC-2 nucleic acid ligand is from about 20 to about 100 nucleotides (nt) in length. In another embodiment, the ligand is from about 30 to about 90, from about 30 to about 40, or from about 30 to about 35 nt in length. In another embodiment, the CLEC-2 nucleic acid ligand is about 30 nt, 31 nt, 32 nt, 33 nt, 34 nt or 35 nt in length.

In one embodiment, the CLEC-2 ligand comprises a nucleic acid sequence that is at least 80%, 85%, 90%, 95%, 98%, 99% or 100% identical to a sequence selected from the group consisting of SEQ ID NOs:4-93, or a pharmaceutically acceptable salt thereof.

In one embodiment, the ligand binds to the protein CLEC-2 or a fragment thereof. In another embodiment, the ligand binds to a soluble domain of CLEC-2. In yet another embodiment, the ligand binds to the CLEC-2 extracellular domain. In still another embodiment, the ligand binds to the CLEC-2 Gln58-Pro230 domain. In one embodiment, the ligand specifically binds to the CLEC-2 protein (SEQ ID NO:1). In another embodiment, the ligand specifically binds to the extracellular domain of CLEC-2 (SEQ ID NO:2).

In one embodiment, the ligand is comprised of nucleotides, wherein one or more of the nucleotides are modified. In another embodiment, nucleotide modifications are stabilizing modifications. In yet another embodiment, the modifications increase stability of the ligand in vitro and/or in vivo. In still another embodiment, the modifications increase bioavailability of the ligand in vivo.

In one embodiment, the ligand comprises an inverted thymine at its 3′ end.

In one embodiment, the ligand has a dissociation constant (“Kd”) for CLEC-2 of about 20 nanomolar (nM) or less.

In one embodiment, the ligand has a dissociation constant for CLEC-2 which is greater than 0 but less than about 100 micromolar (μM), less than about 1 μM, less than about 500 nanomolar (nM), less than about 100 nM, less than about 50 nM, less than about 1 nM, less than about 500 picomolar (pM), less than about 300 pM, less than about 250 pM, or less than about 200 pM. In one embodiment, the ligand has a dissociation constant for CLEC-2 which ranges from about 0.1 to 10 nM or from about 0.5 to about 5 nM.

In one embodiment, the CLEC-2 ligand binds to and decreases or inhibits a function of a variant of CLEC-2, wherein the CLEC-2 variant is at least 80%, 85%, 90%, 91%, 93%, 94%, 95%, 96%, 97%, 98% or 99% identical to the CLEC-2 amino acid sequence (SEQ ID NO: 1 or SEQ ID NO:2).

In one embodiment, binding of the CLEC-2 ligand to CLEC-2 stabilizes an active conformation of CLEC-2. In another embodiment, binding of the CLEC-2 ligand to CLEC-2 stabilizes an inactive conformation of CLEC-2.

In one embodiment, the one or more nucleotides of the ligand comprise a modified sugar and/or a modified base. In another embodiment, the modifications are 2′-stabilizing modifications. In yet another embodiment, the 2′-stabilizing modification is a 2′-fluoro modifications on the nucleotide sugar ring.

In one embodiment, the nucleic acid ligand for CLEC-2 is reversible, wherein the CLEC-2 ligand bound to CLEC-2 can become unbound. In another embodiment, the CLEC-2 ligand bound to CLEC-2 becomes unbound to CLEC-2 in the presence of a CLEC-2 ligand modulator.

In one aspect, a modulator which binds the CLEC-2 ligand is provided, wherein the modulator reverses, partially or completely, the activity of the CLEC-2 ligand.

In one embodiment, the modulator comprises an isolated nucleic acid sequence. In another embodiment, the modulator comprises a DNA sequence, an RNA sequence, a polypeptide sequence, or any combination thereof in one embodiment, the modulator is a nucleic acid modulator comprising deoxyribonucleotides, ribonucleotides, 2′-O-methyl-2′ deoxy nucleotides or a mixture of deoxyribonucleotides and ribonucleotides and 2′-O-methyl-2′ deoxy nucleotides. In another embodiment the nucleic acid modulator comprises at least one modified deoxyribonucleotide and/or at least one modified ribonucleotide.

In one embodiment, the modulator comprises an oligonucleotide which is complementary to at least a portion of the CLEC-2 nucleic acid ligand. In another embodiment, the modulator consists of an oligonucleotide which is complementary to at least a portion of the CLEC-2 nucleic acid ligand. In another embodiment, the modulator comprises an oligonucleotide sequence which is complementary to at least a portion of a loop in the CLEC-2 ligand. In still another embodiment, the modulator comprises an oligonucleotide sequence which is complementary to at least a portion of a stem in the CLEC-2 ligand. In yet another embodiment, the modulator comprises an oligonucleotide sequence which is complementary to at least a portion of a stem in the CLEC-2 ligand and to at least a portion of a loop in the CLEC-2 ligand.

In one embodiment, the modulator comprises a sequence which is greater than 80%, 85%, 90%, 91%, 93%, 94%, 95%, 96%, 97%, 98% or 99% identical to the sequence selected from the group consisting of SEQ liD NO:60, SEQ ID NO:61, SEQ ID NO:62, SEQ ID NO:63, SEQ ID NO:64 and SEQ ID NO:65. In another embodiment, the modulator comprises a sequence selected from the group consisting of SEQ ID NO:60, SEQ ID NO:61, SEQ ID NO:62, SEQ ID NO:63, SEQ ID NO:64 and SEQ ID NO:65.

In one embodiment, the modulator of a CLEC-2 nucleic acid ligand is selected from the group consisting of a ribozyme, a DNAzyme, a peptide nucleic acid (PNA), a morpholino nucleic acid (NINA), and a locked nucleic acid (LNA).

In one embodiment, the modulator of a CLEC-2 nucleic acid ligand comprises a nucleic acid which is complementary to at least a portion of the CLEC-2 nucleic acid ligand. In another embodiment, the modulator is selected from the group consisting of a ribozyme, a DNAzyme, a peptide nucleic acid (PNA), a morpholino nucleic acid (MNA), and a locked nucleic acid (LNA), wherein the modulator specifically binds to or interacts with at least a portion of a CLEC-2 nucleic acid ligand.

In one embodiment, the modulator is selected from the group consisting of a nucleic acid binding protein or peptide, a small molecule, an oligosaccharide, a nucleic acid binding lipid, a polymer, a nanoparticle, and a microsphere, wherein the modulator binds to or interacts with at least a portion of a CLEC-2 nucleic acid ligand.

In one embodiment, the modulator comprises an isolated nucleic acid sequence, wherein the sequence is about 10 nt to about 30 nt, about 10 nt to about 25 nt, about 10 nt to about 20 nt, about 10 nt to about 15 nt, or about 15 nt to about 20 nt in length. In another embodiment, the modulator comprises an isolated nucleic acid sequence, wherein the sequence is about 10 nt, 11 nt, 12 nt, 13 nt, 14 nt, 15 nt, 16 nt, 17 nt, 18 nt, 19 nt, or 20 nt in length.

In one embodiment, one or more of the nucleotides of the CLEC-2 nucleic acid ligand and/or the nucleic acid modulator sequence is modified. In another embodiment, the one or more nucleotides comprise a modification at the 2′ hydroxyl position. In another embodiment, the modification present in the CLEC-2 ligand is selected from the group consisting of 2′-fluoro. In another embodiment, the one or more nucleotides of the CLEC-2 ligand is 2′-fluoro-2′ deoxycytidine or a 2′-fluoro-2′ deoxyuridine. In yet another embodiment, the one or more nucleotides of the modulator is 2′-O-methyl cytosine, 2′-O-methyl uridine, 2′-O-methyl adenosine, 2′-O-methyl guanosine or a 2′-O-methyl thymidine. In still another embodiment, the one or more nucleotides is a 2′ fluoro cytidine, a 2′ fluoro uridine, a 2′ fluoro adenosine or a 2′-fluoro guanosine.

In one embodiment, the modification of one or more nucleotides of the CLEC-2 nucleic acid modulator comprises a modification selected from the group consisting of 5-fluorouracil, 5-fluorocytosine, 5-bromouracil, 5-bromocytosine, 5-chlorouracil, 5-chlorocytosine, 5-iodouracil, 5-iodocytosine, 5-methylcytosine, 5-methylurea, hypoxanthine, xanthine, 4-acetylcytosine, 5-(carboxyhydroxylmethyl)uracil, 5-carboxymethylamino methyl thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 6-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 5-methoxycytosine, 2-methylthio-N6-isopentenyladenine, uracil oxyacetic acid (V), butoxosine, pseudouracil, queosine, 2-thiocytosine, 5-methyl thiouracil, 2-thiouracil, 4-thiouracil, 5-methylurea, uracil-5-oxyacetic acid methylester, uracil oxyacetic acid (v), 5-methyl thiourea, 3-(3-amino-3-N carboxypropyl)uridine (acp3U), and 2,6-diaminopurine.

In one embodiment, the modulator comprises as least one modified sugar moiety.

In one embodiment, the binding of the modulator to the CLEC-2 ligand exposes a suicide position within the CLEC-2 ligand, thereby disrupting the secondary structure of the CLEC-2 ligand and leading to enhanced destruction of the nucleic acid CLEC-2 ligand by nucleases.

In one embodiment, binding of the modulator to a CLEC-2 ligand-CLEC-2 complex reduces or eliminates binding of the CLEC-2 ligand to CLEC-2.

In another aspect, a method of modulating the activity of a CLEC-2 ligand is provided.

In one embodiment, the nucleic acid ligand specifically binds CLEC-2 and inhibits CLEC-2 mediated disorders. In one embodiment, the ligand inhibits the ability of CLEC-2 to mediate platelet aggregation. In one embodiment, the ligand inhibits the ability of CLEC-2 to mediate platelet activation and secretion. In one embodiment, the ligand inhibits the ability of CLEC-2 to propagate thrombus formation. In one embodiment, the ligand inhibits the ability of CLEC-2 to interact with the endogenous protein podoplanin. In one embodiment, the ligand inhibits the ability of CLEC-2 to interact with tumor cells. In one embodiment, the ligand inhibits the ability of CLEC-2 to bind tumor cells via podoplanin and attract platelets to tumor sites. In one embodiment, the ligand inhibits the ability of CLEC-2 from interacting with tumor cells, thereby inhibiting metastasis. In one embodiment, the ligand inhibits the ability of CLEC-2 from interacting with tumor cells, thereby reducing the ability of tumor cells to release growth factors. In one embodiment, the ligand inhibits the ability of CLEC-2 from interacting with Human Immunodeficiency Virus (HIV). In one embodiment, the ligand inhibits the ability of CLEC-2 from facilitating HIV dissemination. In one embodiment, the ligand inhibits the ability of CLEC-2 signaling mediated inflammatory response, cytokine production, platelet-monocyte interaction, and platelet adhesion.

In one embodiment, a method of modulating the activity of a CLEC-2 nucleic acid ligand by administering a modulator of the CLEC-2 ligand to a host who has been administered the nucleic acid CLEC-2 ligand is provided. In one embodiment, the modulator can be an oligonucleotide modulator, or derivative thereof, and in certain embodiments, is complimentary to a portion of the nucleic acid CLEC-2

In a further aspect, a method of regulating CLEC-2 function using a CLEC-2 ligand is provided.

In one embodiment, the method for regulating CLEC-2 function comprises administering to a host a therapeutically effective amount of a CLEC-2 ligand. In another embodiment, the method further comprises administering a CLEC-2 ligand modulator to the host previously administered the CLEC-2

In another aspect, a method of treating or ameliorating a CLEC-2 mediated disease or disorder is provided.

In one embodiment, the method comprises administering to a host in need thereof a therapeutically effective dose of a CLEC-2 ligand that binds to CLEC-2. In one embodiment, the host is diagnosed with a platelet-mediated disorder.

In one embodiment, the platelet-mediated disease or disorder is selected from the group consisting of cardiovascular disorders, cerebrovascular disorders, peripheral vascular disorders, acute coronary syndromes, diabetes-related disorders, and cancer.

In one embodiment, the cerebrovascular disorder is a thrombosis, thromboembolism, or transient ischemia attack (TIA). In another embodiment, the acute coronary syndrome is due to coronary thrombosis, unstable angina or myocardial infarction. In still another embodiment, the diabetes-related disorder is diabetic retinopathy, diabetic vasculopathy, atherosclerosis, ischemic stroke, peripheral vascular disease, acute renal injury or chronic renal failure. In one embodiment, the cancer is selected from lung cancer, breast cancer, prostate cancer, testicular cancer, pancreatic cancer, brain cancer, bone cancer and liver cancer.

In one embodiment, the CLEC-2 ligand is administered by parenteral administration, intravenous injection, intradermal delivery, intra-articular delivery, intra-synovial delivery, intrathecal, intra-arterial delivery, intracardiac delivery, intramuscular delivery, subcutaneous delivery, intraorbital delivery, intracapsular delivery, intraspinal delivery, intrastemal delivery, topical delivery, transdermal patch delivery, rectal delivery, buccal delivery, delivery via vaginal or urethral suppository, peritoneal delivery, percutaneous delivery, delivery via nasal spray, delivery via surgical implant, delivery via internal surgical paint, delivery via infusion pump or delivery via catheter.

In another aspect, a method for treating a host in need thereof by administering a CLEC-2 ligand, wherein the CLEC-2 ligand regulates platelet function is provided.

In one embodiment, a therapeutically effective dose of CLEC-2 is administered.

In one embodiment, the therapeutically effective dose reduces or inhibits platelet adhesion and/or aggregation and/or secretion.

In one aspect, a pharmaceutical composition comprising a therapeutically effective amount of a nucleic acid ligand which binds CLEC-2 is provided.

In one aspect, a pharmaceutical composition comprising a therapeutically effective amount of a modulator, wherein the modulator reverses the activity of a nucleic acid CLEC-2 ligand which binds CLEC-2 is provided.

In one embodiment, the pharmaceutical composition comprises a CLEC-2 ligand and pharmaceutically-acceptable excipients. In another embodiment, the pharmaceutical composition is a liquid suitable for intravenous injection. In yet another embodiment, the pharmaceutical composition is a liquid or dispersion suitable for subcutaneous injection.

In one aspect, a kit comprising a therapeutically effective amount of a CLEC-2 nucleic acid ligand and/or a modulator which regulates the activity of the CLEC-2 nucleic acid ligand is provided.

In one embodiment, the kit comprises a CLEC-2 ligand. In another embodiment, the kit comprises a modulator of a CLEC-2 ligand. In still another embodiment the kit comprises both the CLEC-2 ligand and a modulator for the CLEC-2 ligand.

Methods, pharmaceutical compositions and uses of the nucleic acid ligands described herein are also provided as modulatable therapeutics for use in disorders or treatment regimes requiring anti-platelet or antithrombotic therapies. In certain embodiments, the treatment is a surgical intervention, including percutaneous interventions. The methods can include administering the nucleic acid ligand to CLEC-2 to a host in need thereof, where the host is suffering from, or at risk of suffering from, an occlusive thrombotic disease or disorder of the coronary, cerebral or peripheral vascular system. Additionally, pharmaceutical compositions are provided in which the nucleic acid ligand or its modulator are in combination with a pharmaceutically acceptable carrier. Compositions containing the modulator can be designed for administration to a host who has been given a nucleic acid ligand to allow modulation of the activity of the ligand, and thus regulate the coagulation state of the host at risk of hemorrhage.

In one aspect, a method for determining whether a CLEC-2 ligand activates of inhibits CLEC-2 function is provided. In one embodiment, the CLEC-2 function is CLEC-2-dependent or CLEC-2-mediated platelet aggregation. In another embodiment, a method for determining whether a CLEC-2 ligand activates CLEC-2-dependent platelet aggregation is provided. In still another embodiment, the method is performed in vitro.

In one embodiment, the method comprises

(a) mixing a CLEC-2 ligand with a blood sample to prepare a treated blood sample;

(b) contacting the treated blood sample with a facilitator molecule;

(c) measuring platelet aggregate formation after the contacting; and

(d) comparing the degree of platelet aggregate formation detected in step (c) with the degree of platelet aggregate formation obtained with a control blood sample with no CLEC-2 ligand is used to bind CLEC-2 and inhibit its interaction with the facilitator molecule.

In one embodiment, the facilitator molecule is immobilized to a solid support.

In one embodiment, the facilitator molecule is a CLEC-2 activator. In another embodiment, the facilitator molecule is rhodocytin. In still another embodiment, the facilitator molecule is podoplanin.

In one embodiment, when used in combination with an activator of CLEC-2, the facilitator molecule is a soluble collagen. In another embodiment the soluble collagen is collagen type I, II or III. In still another embodiment, the method further comprises adding a CLEC-2 activator to the blood sample. In yet another embodiment, the CLEC-2 activator is rhodocytin.

In one embodiment, the blood sample is whole blood. In another embodiment, the blood sample is platelet-rich plasma (PRP). In still another embodiment, the blood sample is washed platelets.

In one embodiment, the CLEC-2 ligand is a nucleic acid CLEC-2 ligand. In another embodiment, the nucleic acid CLEC-2 ligand comprises SEQ ID NO:8 or SEQ ID NO:9 or a sequence derived from SEQ ID NO:8 or SEQ ID NO:9.

### DESCRIPTION OF THE FIGURES

FIG. 1 is a diagram of the SELEX nucleic acid ligand selection process.

FIG. 2 shows conditions for Rounds 1-10 of the Sel2 selections.

FIG. 3 shows enrichment of binding of CLEC-2 ligand selections.

FIG. 4 shows binding curves of select CLEC-2 nucleic acid ligands.

FIG. 5 illustrates predicted secondary structures of S2-20 T10 and RB 587.

FIG. 6 shows specific conditions for degenerate selection.

FIG. 7 illustrates S2-20 degenerate selection sequence conservation.

FIG. 8 shows regions of complementarity between a CLEC-2 ligand and modulators.

FIGS. 9A-B show graphs of rhodocytin-induced WP platelet aggregation of CLEC-2 ligands.

FIGS. 10A-C show graphs of rhodocytin-induced platelet aggregation in the presence of CLEC-2 ligands or in the presence of CLEC-ligands and CLEC-2 ligand modulators.

FIGS. 11A-B show graphs of rhodocytin-induced human P-selectin expression.

FIGS. 12A-B show results of in vitro flow-based platelet adhesion assays.

### DETAILED DESCRIPTION

Nucleic acid ligands, also called “aptamers,” are non-naturally occurring, single-stranded nucleic acids that adopt a specific three-dimensional shape which enables binding to a desired target molecule. For ligands which bind to peptides and proteins, association of a ligand with its target protein may lead to the inhibition of the protein's function, much like the binding of a monoclonal antibody to its target protein may lead to the inhibition of the protein's function. A unique feature of nucleic acid ligands is the ability to generate active control agents to them in the form of complementary oligonucleotides that hybridize to the ligand by Watson-Crick basepairing. These active control agents fundamentally change the ligands active structure, and thereby neutralize their pharmacologic activity. The present invention provides compounds, compositions and methods that include nucleic acid ligands to CLEC-2 to mediate the biological function and interaction of CLEC-2. Additionally provided are modulators that can regulate the activity of the CLEC-2 nucleic acid ligands.

### DEFINITIONS

As used herein, the following definitions shall apply unless otherwise indicated.

The term “about”, as used herein when referring to a measurable value such as an amount of weight, time, dose, etc. is meant to encompass variations of ±20% or ±10%, ±5%, ±1%, or ±0.1% from the specified amount, as such variations are appropriate to perform the disclosed method.

A “nucleic acid ligand,” which may also referred to herein as a “ligand” or “aptamer,” is a nucleic acid that can form a tertiary structure, and which interacts with a target molecule. A “CLEC-2 nucleic acid ligand” or “CLEC-2 ligand” or “anti-CLEC-2 ligand” or “nucleic acid CLEC-2 ligand” refers to a ligand or aptamer that specifically binds to CLEC-2 or a fragment thereof. The CLEC-2 fragment may be a soluble fragment, such as the extracellular domain (ECD) or fragment thereof. Alternatively, the CLEC-2 ligand binds a CLEC-2 molecule which is expressed on the surface of a cell or found associated with a platelet. The CLEC-2 protein may be endogenously expressed on a cell or expressed via recombinant means. The terms refer to oligonucleotides having specific binding regions that are capable of forming complexes with an intended target molecule in a physiological environment. The affinity of the binding of a ligand to a target molecule is defined in terms of the dissociation constant (Kd) of the interaction between the ligand and the target molecule. Typically, the Kd of the ligand for its target is between about 0.1 nM to about 100 nM. The specificity of the binding is defined in terms of the comparative dissociation constant of the ligand for target as compared to the dissociation constant with respect to the ligand and other materials in the environment or unrelated molecules in general. Typically, the Kd for the ligand with respect to the target will be 10-fold, 50-fold, 100-fold, or 200-fold less than the Kd with respect to the unrelated material or accompanying material in the environment.

As used herein, in the context of homologous regions, a “substantially homologous” sequence is one that forms the same secondary structure by Watson-Crick base pairing within a particular molecule. In certain embodiments, sequences are “substantially homologous” if they share at least 80%, 85% or more sequence identity, such as about 90%, 91%, 92%, 93%, 94%, 95%, 96%, 97%, 98%, or 99% sequence identity to a specified ligand. For clarity, such “substantially homologous” sequences may also be described as sequences “having at least 80%, 85%, 90%, or 95% sequence identity” to a particular sequence, or that a sequence may be about 90%, 91%, 92%, 93%, 94%, 95%, 96%, 97%, 98%, or 99% identical to a particular ligand sequence. In the context of a nucleic acid ligand of a specified length, such as 50 or less nucleotides, a homologous sequence can be found in any region that allows Watson-Crick binding to form the same secondary structure, regardless of sequence identity within the specific region.

The Sequence Listing filed with this application provides primary sequences only (provides the sequence of nucleotides without particular modifications). It is understood that each of the sequences provided in this specification and in the Sequence Listing as filed may have any combination of modifications as described herein. Sequences having modifications as described in Tables 1-4 are each associated with a specific “clone name” or “name,” which provides a defined description of a nucleic acid sequence with its particularly defined modifications.

“Ligand modulator pair” or “ligand modulator pair” is meant to include a specified ligand to a target molecule, and a ligand modulator that changes the secondary and/or tertiary structure of the ligand so that the ligand's interaction with its target is modulated. The modulator can be an oligonucleotide complimentary to a portion of the ligand. The modulator can change the conformation of the ligand to reduce the target binding capacity of the ligand by 10% to 100%, 20% to 100%, 25%, 40%, 50%, 60%, 70%, 80%, 90% or 100%, or any percentage in the range between 10% and 100% under physiological conditions in vitro, ex vivo or in vivo.

“Modulator,” “antidote,” “regulator” or “control agent” refer to any pharmaceutically acceptable agent that can bind a ligand or aptamer as described herein and modify the interaction between that ligand and its target molecule (e.g., by modifying the structure of the ligand) in a desired manner.

“Modulate” as used herein means a lessening, an increase, or some other measurable change in activity.

“Host” refers to a mammal and includes human and non-human mammals. Examples of host include, but are not limited to mice, rats, hamsters, guinea pigs, pigs, rabbits, cats, dogs, goats, horses, sheep, cows, and humans.

“Pharmaceutically acceptable,” as used herein means approved by a regulatory agency of the Federal or a state government or listed in the U.S. Pharmacopeia or other generally recognized pharmacopeia for use in humans.

A pharmaceutically effective dose is that dose required to prevent, inhibit the occurrence, or treat (alleviate a symptom to some extent) of a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon the potency of the nucleic acid ligand and modulator.

A “stabilized nucleic acid molecule” refers to a nucleic acid molecule that is less readily degraded in vivo (e.g., via an exonuclease or endonuclease) in comparison to a non-stabilized nucleic acid molecule. Stabilization can be a function of length and/or secondary structure and/or inclusion of chemical substitutions within the sugar or phosphate portions of the oligonucleotide backbone. Stabilization can be obtained by controlling, for example, secondary structure which can stabilize a molecule. For example, if the 3′ end of a nucleic acid molecule is complementarily to an upstream region, that portion can fold back and form a “stem loop” structure which stabilizes the molecule.

The terms “binding affinity” and “binding activity” are meant to refer to the tendency of a ligand molecule to bind or not to bind to a target. The energetics of said interactions are significant in “binding activity” and “binding affinity” because they define the necessary concentrations of interacting partners, the rates at which these partners are capable of associating, and the relative concentrations of bound and free molecules in a solution. The energetics may be characterized through, among other ways, the determination of a dissociation constant, Kd.

“Treatment” or “treating” as used herein means any treatment of disease in a mammal, including: (a) protecting against the disease, that is, causing the clinical symptoms not to develop; (b) inhibiting the disease, that is, arresting, ameliorating, reducing, or suppressing the development of clinical symptoms; and/or (c) relieving the disease, that is, causing the regression of clinical symptoms. It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the term “prophylaxis” is intended as an element of “treatment” to encompass both “preventing” and “suppressing” as defined herein. The term “protection,” as used herein, is meant to include “prophylaxis.”

The term “effective amount” means a dosage sufficient to provide treatment for the disorder or disease state being treated. This will vary depending on the patient, the disease and the treatment being effected.

A CLEC-2 nucleic acid ligand “variant” as used herein encompasses variants that perform essentially the same function as a CLEC-2 nucleic acid ligand and comprises substantially the same structure,

### CLEC-2

CLEC-2 is a transmembrane protein expressed on the surface of liver cells and several hematopoietic cells including monocytes, dendritic cells, NK cells, and granulocytes. Of the hematopoietic cells and liver sinusoidal endothelial cells, CLEC-2 protein is specifically expressed in platelets and megakaryocytic cells (Chaipan et al., 2006; J Virol, 80:8951-8960). CLEC-2 has been shown to mediate platelet activation through Src and Syk kinase.

Initially, only exogenous ligands for CLEC-2 had been identified. These included the snake venom, rhodocytin (aggretin), and HIV-1, both of which have been shown to activate platelets and thereby activate platelet aggregation. CLEC-2 deficiency is associated with increased bleeding times and protection from occlusive arterial thrombus formation (May et al., 2009, Blood, 114:3464-3472). May et al. also showed that anti-CLEC-2 antibody treatment of mice leads to complete and highly specific loss of CLEC-2 in circulating platelets for several days, accompanied by severe defects in platelet aggregation.

An endogenous CLEC-2 ligand, recently identified by Ozaki et al. (2010; J. Throm Haemos., 7:191-194), is podoplanin. Podoplanin is a sialoglycoprotein implicated in tumor-induced platelet aggregation and tumor metastasis. Podoplanin is highly expressed in lymphatic endothelium and in kidney podocytes. Expression of podoplanin is not detected in vascular endothelium. The interaction of podoplanin with CLEC-2 is important for the separation of blood and lymphatic vessels during development. Studies have shown that both podoplanin and CLEC-2 deficient mice display a bleeding phenotype, and atypical vascular connections (Schacht, V., et. al., 2003, EMBO, J. 22:3546-3556; Suzuki-Inoue K. et al., 2010, J. Biol. Chem, 285:24494-24507). Studies suggest that CLEC-2 may play a role in hematogenous tumor metastasis as the receptor mediates tumor cell-induced platelet activation, a process known to significantly promote tumor cell spreading (Honn et al., 1992, Cancer Metastasis Rev, 11:325-351; Nieswandt et al., Cancer Res, 1999, 59:1295-1300).

The amino acid sequence of the full-length CLEC-2 protein (GenBank Accession No. AAF36777) is presented below:

| CLEC-2 | |
|---|---|
| (SEQ ID NO: 1) | |
| 1 | MQDEDGYITL NIKTRKPALI SVGSASSSWW RVMALILLIL CVGMVVGLVA LGIWSVMQRN |
| 61 | YLQDENENRT GTLQQLAKRF CQYVVKQSEL KGTFKGHKCS PCDTNWRYYG DSCYGFFRHN |
| 121 | LTWEESKQYC TDMNATLLKI DNRNIVEYIK ARTHLIRWVG LSRQKSNEVW KWEDGSVISE |
| 161 | NMFEFLEDGK GNMNCAYFHN GKMHPTFCEN KHYLMCERKA GMTKVDQLP |

The extracellular domain (ECD) of CLEC-2 encompasses approximately amino acids 58-229 and is presented below:

| CLEC-2(Gln58-Pro229) | |
|---|---|
| (SEQ ID NO 2) | |
| 58 | QRN |
| 61 | YLQDENENRT GTLQQLAKRF CQYVVKQSEL KGTFKGHKCS PCDTNWRYYG DSCYGFFRHN |
| 121 | LTWEESKQYC TDMNATLLKI DNRNIVEYIK ARTHLTRWVG LSRQKSNEVW KWEDGSVISE |
| 181 | NMFEFLEDGK GNMNCAYFHN GKMHPTFCEN KHYLMCERKA GMTKVDQLP |

The extracellular domain (ECD) of CLEC-2 fused to an N-terminal His10 tag (SEQ ID NO:3), which was used to select the nucleic acid ligands described herein, encompasses amino acids 58-229 and is presented below:

HHHHHHHHHHQRNYLQDENENRTGLTLQQLAKRFCQYVVKQSELKGTFKGHKCSPCDTNWRYYGDSCYGFFRHNLTWEESK
QYCTDMNATLLKIDNRNIVEYIKARTHLIRWVGLSRQKSNEVWKWEDGSVISENMFEFLEDGKGNMNCAYFHNGKMHPTF
CENKHYLMCERKAGMTKVDQLP

### Development of CLEC-2 Nucleic Acid Ligands

Nucleic acid ligands which specifically bind the CLEC-2 protein were identified using the SELEX method. The ligands which were initially obtained via SELEX were then fully characterized to understand the properties of the CLEC-2 ligands. Such characterization included sequencing, sequence alignment to determine conserved sequences, secondary structure prediction, and truncations and mutation analysis to identify ligand regions most critical for the desired function of specifically binding and inhibiting CLEC-2.

SELEX refers to the Systematic Evolution of Ligands by EXponential Enrichment. This method allows the in vitro evolution of nucleic acid molecules with highly specific binding to target molecules. The SELEX method is described in, for example, U.S. Pat. Nos. 5,475,096, and 5,270,163 (see also WO 91/19813).

In its most basic form, the SELEX process may be defined by the following series of steps:

1) A candidate mixture of nucleic acids of differing sequence is prepared. The candidate mixture generally includes regions of fixed sequences (i.e., each of the members of the candidate mixture contains the same sequences in the same location) and regions of randomized sequences. The fixed sequence regions are selected either: (a) to assist in the amplification steps described below, (b) to mimic a sequence known to bind to the target, or (c) to enhance the concentration of a given structural arrangement of the nucleic acids in the candidate mixture. The randomized sequences can be totally randomized (i.e., the probability of finding a base at any position being one in four) or only partially randomized (e.g., the probability of finding a base at any location can be selected at any level between 0 and 100%).
2) The candidate mixture is contacted with the selected target under conditions favorable for binding between the target and members of the candidate mixture. Under these circumstances, the interaction between the target and the nucleic acids of the candidate mixture can be considered as forming nucleic acid-target complexes between the target and those nucleic acids having the strongest affinity for the target.
3) The nucleic acids with the highest affinity for the target are partitioned from those nucleic acids with lesser affinity to the target. Because only an extremely small number of sequences (and possibly only one molecule of nucleic acid) corresponding to the highest affinity nucleic acids exist in the candidate mixture, it is generally desirable to set the partitioning criteria so that a significant amount of the nucleic acids in the candidate mixture (approximately 5 to 50%) are retained during partitioning.
4) Those nucleic acids selected during partitioning as having the relatively higher affinity to the target are then amplified to create a new candidate mixture that is enriched in nucleic acids having a relatively higher affinity for the target.
5) By repeating the partitioning and amplifying steps above, the newly formed candidate mixture contains fewer and fewer weakly binding sequences, and the average degree of affinity of the nucleic acids to the target will generally increase. The SELEX process yields a candidate mixture containing one or a small number of unique nucleic acids representing those nucleic acids sequences from the original candidate mixture which fold into a specific secondary and tertiary structure enabling the highest affinity interaction with the target molecule.

Nucleic acid ligands specific to CLEC-2 may be generated by performing SELEX against short peptides which represent the extracellular domain of the molecule, using SELEX methods as described for example in U.S. Pat. No. 7,087,735. Alternatively nucleic acid ligands specific to CLEC-2 can be isolated by performing SELEX on intact platelets, platelet membrane fractions enriched for the protein, on purified CLEC-2, or on cell-lines specifically over-expressing the CLEC-2 receptor using SELEX methods as described, for example, in U.S. Pat. No. 6,730,482.

Additionally, the SELEX process can be directed to isolate specific CLEC-2 nucleic acid ligands using competitive affinity elution schemes, such as those described in U.S. Pat. No. 5,780,228.

In certain embodiments, the CLEC-2 nucleic acid ligand binds to the CLEC-2 receptor under physiological conditions. Physiological conditions are typically related to the level of salts and pH of a solution. In vitro, physiological conditions are typically replicated in a buffer including 150 mM NaCl, 2 mM CaCl₂, 20 mM HEPES, at a pH of about 7.4. In certain embodiments, native, typically unactivated, platelets are used as described above to screen a population of nucleic acid ligands and provide an enriched population, which contains ligands directed to proteins found on platelets. The enriched population is then used against either a stable cell line over-expressing the desired CLEC-2 receptor, or a cell line that has been transiently transfected with the protein. The secondary screening can be accomplished either by using a modified SELEX procedure on isolated receptors from these cells or on the whole cells either through ligand competition studies or by identifying the effects on intracellular signaling pathways.

Ligands can also be screened for inhibition of platelet aggregation in platelet function assays such as Light Transmittance Aggregometry performed in platelet rich plasma or washed platelet preparations or Impedance Aggregometry performed in whole blood or FACS performed in platelet rich plasma or whole blood or washed platelets with activation of platelets by rhodocytin followed by staining with markers of platelet activation and aggregation including anti-CD62 P (P-Selectin), anti-PAC1 (activated GPIIbIIIa) or anti-fibrinogen The specificity of a given nucleic acid ligand for CLEC-2 can be further distinguished by the ability of the ligand to block intracellular signaling events triggered by known agonists of the given receptor. The specificity of a given nucleic acid ligand for CLEC-2 can also be further distinguished by the absence of an effect on platelet aggregation when aggregation is triggered by an agonist that activates a receptor other than CLEC-2.

A ligand as described herein is comprised of an isolated nucleic acid sequence, which can be DNA or RNA, and which can be synthesized using modified ribo- or deoxyribonucleic acids. In certain embodiments described herein, the sequence of nucleic acids is written as an RNA sequence. Similarly, in certain embodiments described herein, wherein the nucleic acid ligand is initially identified as a DNA molecule, the sequence of nucleic acids is written as a DNA sequence. It is understood that a sequence of nucleotides presented in text form as a DNA sequence inherently provides description of the corresponding RNA sequence, wherein thymines (T's) within the DNA sequence are replaced with uridines (Us) to get the corresponding RNA sequence of nucleotides. Similarly, it is understood that a sequence presented in text form as a RNA sequence inherently provides description of the corresponding DNA sequence, wherein uridines (U's) within the RNA sequence are replaced with thymines (T's) to get the corresponding DNA sequence.

The binding affinity of the ligands with respect to the target can be defined in terms of Kd. The value of this dissociation constant can be determined directly by well-known methods, such as by radioligand binding methods described in Example 1.

In some embodiments, the Kd of binding of the ligand to CLEC-2 can range from between about 1 nM to about 100 nM, from about 10 nM to about 50 nM or from about 0.1 nM to about 20 nM. In other embodiments, the Kd of binding of a ligand to CLEC-2 is at least 2-fold, 3-fold, 4-fold, 5-fold or 10-fold less than the Kd of binding of the ligand to an unrelated protein or other accompanying material in the environment. The unrelated protein could also be a protein having motifs related to those present in CLEC-2, such as another platelet activation or adhesion receptor.

As will be discussed in greater detail below, the binding activity of the ligand obtained and identified by the SELEX method can be further modified or enhanced using a variety of engineering methods.

In some embodiments, the ligand interacts with the extracellular domain of CLEC-2. The ligand can interfere with binding of endogenous ligands to the CLEC-2 receptor. In certain embodiments, the ligand can inhibit intracellular signaling via the CLEC-2 receptor. The ligand can also stabilize or disrupt a conformation of the receptor, such as a dimeric conformation, so that the receptor has a reduced capacity to interact with an endogenous ligand such as podoplanin. In certain embodiments the ligand can affect platelet aggregation and/or activation and/or platelet adhesion and/or platelet secretion.

The nucleic acid ligands described herein can function as actively reversible agents. These are agents or pharmaceutically active molecules that, after administration to a patient, can be directly controlled by the administration of a second agent. As described in more detail below, the second agent, referred to herein as a modulator, can shut off or fine-tune the pharmacologic activity of the ligand. As a result, the pharmacologic activity of the ligand can be reversed by means other than, for example, drug clearance.

The CLEC-2 ligands disclosed herein are preferably nucleic acid ligands which specifically bind the CLEC-2 extracellular domain (ECD) (amino acid residues Gln58-Pro230; SEQ ID NO:2). CLEC-2 ligands were selected using the SELEX method, described in more detail below and in Example 1, then modified to increase stability, affinity for CLEC-2 and/or the ability to regulate CLEC-2 activity.

Ligands isolated according to methods described herein are presented below in Tables 1-2, presented herein in Example 2.

As described herein, aptamers which bind CLEC-2 were isolated and sequenced as described in Examples 1-2, resulting in the identification of 20 unique sequences. One clone, S2-20, was identified as a high affinity inhibitor of CLEC-2-dependent platelet aggregation. This clone was characterized further by the generation of truncated and/or mutated versions of the SELEX-selected sequence as described in Examples 3-5. Secondary structure analysis of the clone S2-20 (SEQ ID NO: 8 or SEQ ID NO:9), using software available at the mfold server (mfold.bioinfo.rpi.edu), predicted a consensus structure wherein the CLEC-2 ligand (aptamer) have a first stem about 6 bp in length, but data show that the length of this first stem can range from 5 to 10 bp and maintain high affinity for CLEC-2. Thus, the first stem of the aptamer may range from about 3 bp to about 15 bp, or from about 4 bp to about 12 bp, or from about 5 bp to about 10 bp. It is envisioned that the first stem may have a length of about 3 bp, 4 bp, 5 bp, 6 hp, 7 bp, 8 bp, 9 bp, 10 bp, 11 bp, 12 bp, 13 bp, 14 bp or 15 bp. The CLEC-2 ligand structure was also predicted to have a first loop having the sequence 5′-GAC-3′, though mutational analysis showed tolerance of mutations in the second position of the first loop structure. Accordingly, it is envisioned that the first loop of the CLEC-2 ligand comprises a 5′-GNC-3′ consensus sequence, wherein N can be A, T, C, G or U. 3′ to the first loop is a second stem, having a length of about 4 bp with a wobble base pair at the base of the stem, though this length may vary from about 3 bp to 7 bp or 4 bp to about 6 bp. 3′ to the second stem is a second loop structure with the sequence 5′-CUCAUAUU-3′. Mutational analysis showed that a preferred loop 2 consensus sequence may be 5′-YUYNNRYU′-3′, wherein Y is a pyrimidine, R is a purine and N is A, G, C, T or U. Of the truncated CLEC-2 ligands derived from S2-20, S2-20 T10 SEQ ID NO:22) and RB587 (SEQ ID NO:24) are examples of such ligands.

Determination of a consensus structure facilitates engineering of ligands to identify one or more nucleotides which may enhance or decrease ligand structure and function. It allows one to more efficiently identify and test nucleotide additions, deletions and substitutions to specific stem and loop structures.

Knowledge of a consensus secondary structure also allows one to avoid modifications which may be detrimental to ligand structure and function. For example, certain modifications may be conserved within the consensus secondary structure, such as a 2′-fluoro within a stem or loop region. In these instances, removal of a 2′-fluoro from the stem or loop of an ligand may result in the loss of activity.

In one embodiment, the ligand is a nucleic acid molecule selected using the SELEX method and include truncates and substantially homologous sequences thereof. As used herein, in the context of homologous regions, a “substantially homologous” sequence is one that forms the same secondary structure by Watson-Crick base pairing within a particular molecule. In another embodiment, the ligand selected using the SELEX method is selected from the group consisting a nucleic acid sequence which comprises a nucleic acid sequence that is at least 80%, 85%, 90%, 95%, 96%, 97%, 98%, or 99% identical or is identical to SEQ ID NOs:5-59, or to a sequence selected from the group consisting of SEQ NOs:9-14, 16-22, 24, 33, 37, 38, 42, 44-48, and 55-57. In still another embodiment, the ligand is one which has a dissociation constant of less than about 10 nM or less than about 5 nM as measured in vitro under physiological conditions.

CLEC-2 ligands described herein may be nucleic acid molecules which comprise a sequence which differs from the S2-20 sequence by having a deletion at one or more nucleotide positions within SEQ NO:7, SEQ ID NO:8 or SEQ ID NO:9. A CLEC-2 ligand may be produced by introducing any combination of one or more deletions within SEQ ID NO:7, SEQ ID NO:8 or SEQ NO:9.

The CLEC-2 ligand referred to herein may comprise a sequence generated by truncating (deleting) one or more nucleotides from the 5′ and/or 3′ terminus of SEQ ID NO:7, SEQ ID NO:8 or SEQ ID NO:9. For example, a CLEC-2 ligand may be a sequence generated by deleting the first (5′) 1, 2, 3, 4, 5, 6, 7, 8, 9, 10, 11, 12, 13, 14, 15, or 16 nucleotides of SEQ ID NO:7, SEQ ID NO:8 or SEQ ID NO:9. In an alternative embodiment, the last (3′) 1, 2, 3, 4, 5, 6, 7, 8, 9, 10, 11, 12, 13, 14, 15, or 16 nucleotides are deleted from SEQ ID NO: 7, SEQ ID NO:8 or SEQ ID NO:9. In still another embodiment, the nucleotides at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 or 80, or any combination thereof are deleted from the sequence of SEQ ID NO: 7, SEQ ID NO:8 or SEQ ID NO:9. In another embodiment, the combination of deleted sequence consists of 1, 2, 3, 4, 5, 6, 7, 8, 9, or 10 nucleotides.

In a preferred embodiment, the CLEC-2 ligand comprises a sequence which is at least 80%, 85%, 90%, or 95% identical to SEQ ID NO:24. In this embodiment, the CLEC-2 ligand has a length ranging from about 20 bp to 40 bp, 25 bp to 30 bp, or about 27 bp to 33 bp. This CLEC-2 ligand has a secondary structure comprising a first stem, a first loop comprising 5′-GNC-3′ (wherein N is 6, A, T, C or U), a second stem and a second loop comprising 5′-CUCAUAUU-3′ or 5′-YUYNNRYU′-3′ (wherein Y is a pyrimidine, R is a purine and N is A, G, C, T or U). In the context of a nucleic acid ligand SEQ ID NO:24, a homologous sequence can be found in any region that allows Watson-Crick binding to form the same secondary structure, regardless of sequence identity within the specific region.

The CLEC-2 ligand as described herein functions to inhibit the activity of the CLEC-2 molecule in vitro and/or in vivo. In other words, addition of the CLEC-2 ligand may reduce CLEC-2 activity in a specified assay designed to measure CLEC-2 activity. For example, the CLEC-2 ligand inhibits CLEC-2 function. A CLEC-2 ligand may function by inhibiting CLEC-2-dependent platelet functions in vitro and/or in vivo.

### Modulators of CLEC-2 Nucleic Acid Ligands

In some embodiments, the nucleic acid ligands to CLEC-2 are reversible. In one aspect, provided is a method for modulating the activity of a ligand to CLEC-2 by administering a modulator of the CLEC-2 ligand to a host who has been administered the nucleic acid ligand.

Modulators can include any pharmaceutically acceptable agent that can bind to a nucleic acid ligand and modify the interaction between that ligand and its target molecule (e.g., by modifying the structure of the nucleic acid ligand) in a desired manner, or which degrades, metabolizes, cleaves, or otherwise chemically alters the nucleic acid ligand to modify its biological effect.

Nucleic acid modulators of the CLEC-2 ligands described herein are designed based on standard complementary Watson-Crick basepairing rules (see Example 6). Varying lengths of modulatory oligonucleotides are synthesized to have sequences complementary to a CLEC-2 ligand which has been shown to inhibit CLEC-2 function. Each of the synthesized modulator oligonucleotides are then tested for its ability to bind to the CLEC-2 ligand, e.g., via a gel mobility shift assay. It is important to then test whether or not the modulators which bind a CLEC-2 ligand are also capable of reversing the activity of the CLEC-2 ligand. As described in more detail below and in the Examples section, the reversing activity of a given modulator can be tested using assays which evaluate antiplatelet activity of CLEC-2 ligands. Assays performed to identify CLEC-2 ligands which inhibit CLEC-2-mediated platelet aggregation are also performed in the presence of modulators which have been shown to bind the CLEC-2 ligand. Modulators may be added to the assay mixture before, concurrent with, or after addition of the CLEC-2 ligand. The reversing activity of a modulator can be demonstrated using, for example, washed platelet aggregation studies, rhodocytin- or podoplanin-induced platelet aggregation assays in whole blood, platelet-rich plasma, or in washed platelet preparations, flow cytometry assays performed with rhodocytin or podoplanin to assess P-selectin expression in the presence of the CLEC-2 ligand, and in vitro flow based platelet adhesion assays.

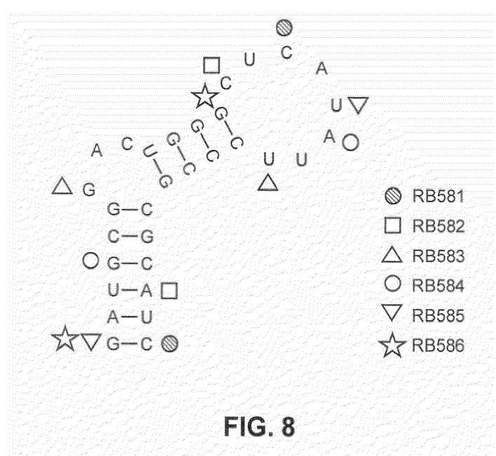

In a preferred embodiment, the modulator of the CLEC-2 nucleic acid ligand is a nucleic acid sequence which is at least partly complementary to the CLEC-2 nucleic acid ligand sequence. As shown in FIG. 8 (and see Table 4), modulators of the CLEC-2 ligand RB587 were designed to hybridize to different regions of RB587. RB581, which is complementary to the 3′ 16 bases of the primary sequence of RB587, was effective in reducing binding of RB587 to CLEC-2 as determined by neutralization of CLEC-2 ligand inhibitory activity in platelet aggregation assays and as determined by gel mobility shift assays. Thus, in some embodiments, a modulator which is complementary or is able to hybridize to the same region of a CLEC-2 ligand is provided herein. As such, a modulator as described herein is able to hybridize to the region of the CLEC-2 nucleic acid ligand encompassing loop 2, stem 2 and stem 1, or a portion thereof. As one specific example, a modulator as described herein is able to hybridize to the region corresponding to the bases at approximately positions 14-31 or 14-26 or 16-31 or 18-31 or 21-31 or 16-26 or 16-29 of the CLEC-2 ligand RB587 (SEQ ID NO:24). Moreover, specific interaction of the modulator with the CLEC-2 ligand results in a reduction of binding of the CLEC-2 ligand to a CLEC-2 polypeptide. Moreover, the modulator may inhibit or reverse the inhibition of the CLEC-2 target protein function by the CLEC-2

In a preferred embodiment, the modulator of the CLEC-2 nucleic acid ligand is a nucleic acid sequence which is at least partly complementary to the CLEC-2 nucleic acid ligand sequence. As shown in FIG. 8 (and see Table 4), modulators of the CLEC-2 RB587 were designed to hybridize to different regions of RB587. RB3582, which is complementary to an internal 16 bases of the primary sequence of RB587, was effective in reducing binding of RB587 to CLEC-2 as determined by neutralization of CLEC-2 ligand inhibitory activity in platelet aggregation assays and as determined by get mobility shift assays. Thus, in some embodiments, a modulator which is complementary or is able to hybridize to the same region of a CLEC-2 ligand is provided herein. As such, a modulator as described herein is able to hybridize to the region of the CLEC-2 nucleic acid ligand encompassing loop 2, stem 2 and a portion of stem 1, or a portion thereof. As one specific example, a modulator as described herein is able to hybridize to the region corresponding to the bases at approximately positions 14-31 or 14-29 or 14-27 or 16-29 or 16-27 or 19-29 or 21-31 or 21-29 of the CLEC-2 ligand RB3587 (SEQ ID NO:24). Moreover, specific interaction of the modulator with the CLEC-2 ligand results in a reduction of binding of the CLEC-2 ligand to a CLEC-2 polypeptide. Moreover, the modulator may inhibit or reverse the inhibition of the CLEC-2 target protein function by the CLEC-2 ligand.

In a preferred embodiment, the modulator of the CLEC-2 nucleic acid ligand is a nucleic acid sequence which is at least partly complementary to the CLEC-2 nucleic acid ligand sequence. As shown in FIG. 8 (and see Table 4), modulators of the CLEC-2 ligand RB587 were designed to hybridize to different regions of RB587. RB583, which is complementary to an internal 15 bases of the primary sequence of RB587, was effective in reducing binding of RB587 to CLEC-2 as determined by neutralization of CLEC-2 ligand inhibitory activity in platelet aggregation assays and as determined by get mobility shift assays. Thus, in some embodiments, a modulator which is complementary or is able to hybridize to the same region of a CLEC-2 ligand is provided herein. As such, a modulator as described herein is able to hybridize to the region of the CLEC-2 nucleic acid ligand encompassing loop 1, stem 2 and loop2, or a portion thereof. As one specific example, a modulator as described herein is able to hybridize to the region corresponding to the bases at approximately positions 7-21 or 9-21 or 9-17 or 7-19 or 7-17 or 7-14 of the CLEC-2 ligand RB587 (SEQ ID NO:24). Moreover, specific interaction of the modulator with the CLEC-2 ligand results in a reduction of binding of the CLEC-2 ligand to a CLEC-2 polypeptide. Moreover, the modulator may inhibit or reverse the inhibition of the CLEC-2 target protein function by the CLEC-2 ligand.

In a preferred embodiment, the modulator of the CLEC-2 nucleic acid ligand is a nucleic acid sequence which is at least partly complementary to the CLEC-2 nucleic acid ligand sequence. As shown in FIG. 8 (and see Table 4), modulators of the CLEC-2 ligand RB587 were designed to hybridize to different regions of RB587. RB584, which is complementary to an internal 16 bases of the primary sequence of RB587, was effective in reducing binding of RB587 to CLEC-2 as determined by neutralization of CLEC-2 ligand inhibitory activity in platelet aggregation assays and as determined by gel mobility shift assays. Thus, in some embodiments, a modulator which is complementary or is able to hybridize to the same region of a CLEC-2 ligand is provided herein. As such, a modulator as described herein is able to hybridize to the region of the CLEC-2 nucleic acid ligand encompassing a portion of stem 1, loop 1, stem 2 and a portion of loop 2, or a portion thereof. As one specific example, a modulator as described herein is able to hybridize to the region corresponding to the bases at approximately positions 1-21 or 1-19 or 1-14 or 4-14 or 4-19 or 4-21 or 6-21 or 6-14 of the CLEC-2 ligand RB587 (SEQ ID NO:24). Moreover, specific interaction of the modulator with the CLEC-2 ligand results in a reduction of binding of the CLEC-2 ligand to a CLEC-2 polypeptide. Moreover, the modulator may inhibit or reverse the inhibition of the CLEC-2 target protein function by the CLEC-2 ligand.

In a preferred embodiment, the modulator of the CLEC-2 nucleic acid ligand is a nucleic acid sequence which is at least partly complementary to the CLEC-2 nucleic acid ligand sequence. As shown in FIG. 8 (and see Table 4), modulators of the CLEC-2 ligand RB587 were designed to hybridize to different regions of RB587. RB585, which is complementary to the 5′ 18 bases of the primary sequence of RB587, was effective in reducing binding of RB587 to CLEC-2 as determined by neutralization of CLEC-2 ligand inhibitory activity in platelet aggregation assays and as determined by gel mobility shift assays. Thus, in some embodiments, a modulator which is complementary or is able to hybridize to the same region of a CLEC-2 ligand is provided herein. As such, a modulator as described herein is able to hybridize to the region of the CLEC-2 nucleic acid ligand encompassing stem 1, loop 1, stem 2 and a portion of loop 2, or a portion thereof. As one specific example, a modulator as described herein is able to hybridize to the region corresponding to the bases at approximately positions 1-21 or 1-18 or 1-16 or 1-14 or 4-21 or 4-18 or 4-14 or 6-14 or 6-18 or 6-21 of the CLEC-2 ligand RB587 (SEQ ID NO:24). Moreover, specific interaction of the modulator with the CLEC-2 ligand results in a reduction of binding of the CLEC-2 ligand to a CLEC-2 polypeptide. Moreover, the modulator may inhibit or reverse the inhibition of the CLEC-2 target protein function by the CLEC-2 ligand.

In a preferred embodiment, the modulator of the CLEC-2 nucleic acid ligand is a nucleic acid sequence which is at least partly complementary to the CLEC-2 nucleic acid ligand sequence. As shown in FIG. 8 (and see Table 4), modulators of the CLEC-2 ligand RB587 were designed to hybridize to different regions of RB587. RB586, which is complementary to the 5′ 13 bases of the primary sequence of RB587, was effective in reducing binding of RB587 to CLEC-2 as determined by neutralization of CLEC-2 ligand inhibitory activity in platelet aggregation assays and as determined by gel mobility shift assays. Thus, in some embodiments, a modulator which is complementary or is able to hybridize to the same region of a CLEC-2 ligand is provided herein. As such, a modulator as described herein is able to hybridize to the region of the CLEC-2 nucleic acid ligand encompassing stem 1, loop 1 and stem 2, or a portion thereof. As one specific example, a modulator as described herein is able to hybridize to the region corresponding to the bases at approximately positions 1-13 or 1-10 or 6-13 of the CLEC-2 ligand RB587 (SEQ ID NO:24). Moreover, specific interaction of the modulator with the CLEC-2 ligand results in a reduction of binding of the CLEC-2 ligand to a CLEC-2 polypeptide. Moreover, the modulator may inhibit or reverse the inhibition of the CLEC-2 target protein function by the CLEC-2 ligand.

Alternative Examples of modulators include: oligonucleotides, or analogues thereof, that are complementary to at least a portion of the nucleic acid ligand sequence (including ribozymes or DNAzymes). Other examples include peptide nucleic acids (PNA), mopholino nucleic acids (MNA), or locked nucleic acids (LNA); nucleic acid binding proteins or peptides; oligosaccharides; small molecules; or nucleic acid binding polymers, lipids, nanoparticle, or microsphere-based modulators.

Modulators can be designed so as to bind a particular nucleic acid ligand with a high degree of specificity and a desired degree of affinity. Modulators can also be designed so that, upon binding, the structure of the ligand is modified to either a more or less active form. For example, the modulator can be designed such that upon binding to the targeted nucleic acid ligand, the secondary and/or tertiary structure of that ligand is altered whereby the ligand can no longer bind to its target molecule or binds to its target molecule with less affinity. Alternatively, the modulator can be designed so that, upon binding, the three dimensional structure of the ligand is altered so that the affinity of the ligand for its target molecule is enhanced. That is, the modulator can be designed so that, upon binding, a structural motif is modified such that affinity of the ligand is increased. In another embodiment, a ligand/modulator pair is designed such that binding of the modulator to a nucleic acid ligand molecule, which cannot bind to the target of interest, can result in production of a structural motif within the ligand which thereby allows the ligand to bind to its target molecule.

Modulators can also be designed to nonspecifically bind to a particular nucleic acid ligand or set of nucleic acid ligand with sufficient affinity to form a complex. Such modulators can generally associate with nucleic acids via charge-charge interactions. Such modulators can also simultaneously bind more than one nucleic acid ligand. The modulator can be designed so that, upon binding to one or more nucleic acid ligands, the structure of the nucleic acid ligand is not significantly changed from its active form, but rather, the modulator masks or sterically prevents association of the nucleic acid ligand with its target molecule.

Nucleotide modulators can be of any length that allows effective binding to the ligand molecule. For example, oligonucleotide modulators can range in length from about 10 nucleotides (nt) to about 30 nt, from about 10 nt to about 20 nt, or from about 15 nt. The nucleotide modulators may be 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 in, 25 nt, 26 nt, 27 nt, 28 nt, 29 in or 30 nt in length. One having ordinary skill in the art can also envision nucleotide modulators having lengths greater than 30 nt.

A nucleic acid ligand as described herein possesses an active tertiary structure, which can be affected by formation of the appropriate stable secondary structure. Therefore, while the mechanism of formation of a duplex between a complementary oligonucleotide modulator and a nucleic acid ligand is similar to formation of a duplex between two short linear oligoribonucleotides, both the rules for designing such interactions and the kinetics of formation of such a product can be impacted by the intramolecular ligand structure.

The rate of nucleation of initial basepair formation between the nucleic acid ligand and oligonucleotide modulator plays a significant role in the formation of the final stable duplex, and the rate of this step is greatly enhanced by targeting the oligonucleotide modulator to single-stranded loops and/or single-stranded 3′ or 5′ tails present in the nucleic acid ligand. For the optimal formation of the intermolecular duplex to occur, the free energy is ideally favorable to the formation of the intermolecular duplex with respect to formation of the existing intramolecular duplexes within the targeted nucleic acid ligand.

The modulators described herein are generally oligonucleotides which comprise a sequence complementary to at least a portion of the targeted nucleic acid ligand sequence. For example, the modulator oligonucleotide can comprise a sequence complementary to about 6 nt (nucleotides) to 25 nt, 8 n1 in 20 nt, or 10 nt to 15 nt of the targeted ligand. The length of the modulator oligonucleotide can be readily optimized using techniques described herein and known to persons having ordinary skill in the art, taking into account the targeted ligand and the effect sought. The oligonucleotide can be made with nucleotides bearing D or L stereochemistry, or a mixture thereof. Naturally occurring nucleosides are in the configuration.

While the oligonucleotide modulators include a sequence complementary to at least a portion of a nucleic acid ligand, absolute complementarity is not required. A sequence “complementary to at least a portion of a nucleic acid ligand,” referred to herein, is a sequence having sufficient complementarity to be able to hybridize with the nucleic acid ligand. The ability to hybridize can depend on both the degree of complementarity and the length of the nucleic acid. Generally, the larger the hybridizing oligonucleotide, the more base mismatches with a target ligand it can contain and still form a stable duplex (or triplex as the case may, be). One skilled in the art can ascertain a tolerable degree of mismatch by use of standard procedures to determine the melting point of the hybridized complex. The oligonucleotides can be single-stranded DNA or RNA or chimeric mixtures or derivatives or modified versions thereof.

The modulators can include modifications in both the nucleic acid backbone and structure of individual nucleic acids. In certain embodiments, the modulator is a nucleic acid complementary to at least one loop region in the ligand. In other embodiments, the modulator is a nucleic acid complementary to at least one stem region in the ligand. In yet other embodiments, the modulator is a nucleic acid complementary to at least one loop region and one contiguous stem region. In some embodiments, the modulator is an oligonucleotide having at least a sequence that hybridizes at physiological conditions to a portion of the ligand. Depending on the desired function of the modulator, the modulator can be designed to disrupt or stabilize the secondary and/or tertiary structure of the nucleic acid ligand.

In some embodiments, the modulator is designed to bind to a “suicide position” on the ligand and thereby disrupt the sequence of the ligand. A suicide position is a single stranded portion of the ligand susceptible to enzymatic cleavage. In one exemplary embodiment, the suicide position becomes single stranded and labile upon binding of the modulator to the ligand and can enhance cleavage of the ligand by enzymes in the circulation, such as blood or liver endonucleases. In certain embodiments, the modulator binds to the ligand after which the ligand can no longer interact with its target.

In some embodiments, a modulator sequence comprises at least one modified nucleotide. For example, a 2′-O-methyl and 2′-fluoro modification, which can include 2′-O-methyl cytosine, 2′-O-methyl uridine, 2′-O-methyl adenosine, 2′-O-methyl guanosine, 2′ fluoro cytidine, or 2′ fluoro uridine.

Various strategies can be used to determine the optimal site within a nucleic acid ligand for binding by an oligonucleotide modulator. An empirical strategy can be used in which complimentary oligonucleotides are “walked” around the nucleic acid ligand. In accordance with this approach, oligonucleotides (e.g., 2′-O-methyl or 2′-fluoro oligonucleotides) about 15 nucleotides in length can be used that are staggered by about 5 nucleotides on the ligand (e.g., oligonucleotides complementary to 1-15, 6-20, 11-25, etc. of ligand). An empirical strategy can be particularly effective because the impact of the tertiary structure of the nucleic acid ligand on the efficiency of hybridization can be difficult to predict.

Assays, such as those described in Example 6 and 8, can be used to assess the ability of the different oligonucleotides to hybridize to a specific nucleic acid ligand, with particular emphasis on the molar excess of the oligonucleotide required to achieve complete binding of the nucleic acid ligand. The ability of the different oligonucleotide modulators to increase the rate of dissociation of the nucleic acid ligand from, or association of the ligand with, its target molecule can also be determined by conducting standard kinetic studies using, for example, BIACORE assays. Oligonucleotide modulators can be selected such that a 5-50 fold molar excess of oligonucleotide, or less, is required to modify the interaction between the ligand and its target molecule in the desired manner.

Alternatively, the targeted nucleic acid ligand can be modified so as to include a single-stranded tail (3′ or 5′) in order to promote association with an oligonucleotide modulator. Suitable tails can comprise 1 to 20 nucleotides, 1 to 10 nucleotides, 1 to 5 nucleotides or 3 to 5 nucleotides. Tails may also be modified (e.g., a 2′-O-methyl and 2′-fluoro modification, which can include 2′-O-methyl cytosine, 2′-O-methyl uridine, 2′-O-methyl adenosine, 2′-O-methyl guanosine, 2′ fluoro cytidine, or 2′ fluoro uridine). Tailed ligands can be tested in binding and bioassays as described in the Examples that follow) to verify that addition of the single-stranded tail does not disrupt the active structure of the nucleic acid ligand. A series of oligonucleotides (for example, 2′-O-methyl oligonucleotides) that can form, for example, 1, 2, 3, 4 or 5 base pairs with the tail sequence can be designed and tested for their ability to associate with the tailed ligand alone, as well as their ability to increase the rate of dissociation of the ligand from, or association of the ligand with, its target molecule. Scrambled sequence controls can be employed to verify that the effects are due to duplex formation and not non-specific effects.

In another embodiment, the modulator is a ribozyme or a DNAzyme. Enzymatic nucleic acids act by first binding to a target RNA or DNA. Such binding occurs through the target binding portion of an enzymatic nucleic acid which is held in close proximity to an enzymatic portion of a molecule that acts to cleave the target RNA. Thus, the enzymatic nucleic acid first recognizes and then binds a target RNA or DNA through complementary base-pairing, and once bound to the correct site, acts enzymatically to cut the target RNA, thereby allowing for inactivation of RNA ligands. There are at least five classes of ribozymes that each display a different type of specificity. For example, Group Introns are about 300 to >1000 nucleotides in size and require a U in the target sequence immediately 5′ of the cleavage site and binds 4-6 nucleotides at the 5′-side of the cleavage site. Another class is RNaseP RNA (M1 RNA), which are about 290 to 400 nucleotides in size. A third class is Hammerhead Ribozymes, which are about 30 to 40 nucleotides in size. They require the target sequence UH (where H is not G) immediately 5′ of the cleavage site and bind a variable number of nucleotides on both sides of the cleavage site. A fourth class is the Hairpin Ribozymes, which are about 50 nucleotides size. They require the target sequence GUC immediately 3° of the cleavage site and bind 4 nucleotides at the 5′-side of the cleavage site and a variable number to the 3′-side of the cleavage site. A fifth group is Hepatitis Delta Virus (HDV) Ribozymes, which are about 60 nucleotides in size. DNAzymes are single-stranded, and cleave both RNA and DNA. A general model for the DNAzyme has been proposed, and is known as the “10-23” model, DNAzymes following the “10-23” model have a catalytic domain of 15 deoxyribonucleotides, flanked by two substrate-recognition domains of seven to nine deoxyribonucleotides each.

In another embodiment, the modulator itself is a nucleic acid ligand. In this embodiment, a first ligand is generated that binds to the desired therapeutic target. In a second step, a second ligand that binds to the first ligand is generated using the SELEX process described herein or another process, and modulates the interaction between the therapeutic ligand and the target. In one embodiment, the second ligand deactivates the effect of the first ligand.

In another exemplary embodiment, the modulator is a PNA, MNA, LNA, or PCO based modulator. Nucleobases of the oligonucleotide modulators can be connected via internucleobase linkages, e.g., peptidyl linkages (as in the case of peptide nucleic acids (PNAs); Nielsen et al. (1991) Science 254, 1497 and U.S. Pat. No. 5,539,082) and morpholino linkages (Qin et al., Antisense Nucleic Acid Drug Dev. 10, 11 (2000); Summerton, Antisense Nucleic Acid Drug Dev. 7, 187 (1997); Summerton et al., Antisense Nucleic Acid Drug Dev. 7, 63 (1997); Taylor et al., J Biol. Chem. 271, 17445 (1996); Partridge et al., Antisense Nucleic Acid Drug Dev, 6, 169 (1996)), or by any other natural or modified linkage. The oligonucleobases can also be Locked Nucleic Acids (LNAs). Nielsen et al., J Biomol Struct Dyn 17, 175 (1999); Petersen et al., J Mol Recognit 13, 44 (2000); Nielsen et al., Bioconjug Chem 11, 228 (2000).

PNAs are compounds that are analogous to oligonucleotides, but differ in composition. PNAs, the deoxyribose backbone of oligonucleotide is replaced with a peptide backbone. Each subunit of the peptide backbone is attached to a naturally-occurring or non-naturally-occurring nucleobase. PNA often has an achiral polyamide backbone consisting of N-(2-aminoethyl)glycine units. The purine or pyrimidine bases are linked to each unit via a methylene carbonyl linker (1-3) to target the complementary nucleic acid. PNA binds to complementary RNA or DNA in a parallel or antiparallel orientation following the Watson-Crick base-pairing rules. The uncharged nature of the PNA oligomers enhances the stability of the hybrid PNA/DNA(RNA) duplexes as compared to the natural homoduplexes.

Morpholino nucleic acids are so named because they are assembled from morpholino subunits, each of which contains one of the four genetic bases (adenine, cytosine, guanine, and thymine) linked to a 6-membered morpholine ring. Subunits of these four subunit types are joined in a specific order by non-ionic phosphorodiamidate intersubunit linkages to give a morpholino oligo.

LNA is a class of DNA analogues that possess some features that make it a prime candidate for modulators. The LNA monomers are bi-cyclic compounds structurally similar to RNA-monomers. LNA shares most of the chemical properties of DNA and RNA, it is water-soluble, can be separated by gel electrophoreses, ethanol precipitated etc (Tetrahedron, 54, 3607-3630 (1998)). However, introduction of LNA monomers into either DNA or RNA oligos results in high thermal stability of duplexes with complementary DNA or RNA, while, at the same time obeying the Watson-Crick base-pairing rules.

Pseudo-cyclic oligonucleobases (PCOs) can also be used as a modulator (see U.S. Pat. No. 6,383,752). PCOs contain two oligonucleotide segments attached through their 3′-3′ or 5′-5′ ends. One of the segments (the “functional segment”) of the PCO has some functionality (e.g., complementarity to a target RNA). Another segment (the “protective segment”) is complementary to the 3′- or 5′-terminal end of the functional segment (depending on the end through which it is attached to the functional segment). As a result of complementarity between the functional and protective segment segments, PCOs form intramolecular pseudo-cyclic structures in the absence of the target nucleic acids (e.g., RNA), PCOs are more stable than conventional oligonucleotides because of the presence of 3′-3′ or 5′-6′ linkages and the formation of intramolecular pseudo-cyclic structures. Pharmacokinetic, tissue distribution, and stability studies in mice suggest that PCOs have higher in vivo stability than and, pharmacokinetic and tissue distribution profiles similar to, those of PS-oligonucleotides in general, but rapid elimination from selected tissues. When a fluorophore and quencher molecules are appropriately linked to the PCOs of the present disclosure, the molecule will fluoresce when it is in the linear configuration, but the fluorescence is quenched in the cyclic conformation. This feature can be used to screen PCO's as potential modulators.

In another exemplary embodiment, the modulators are peptide-based modulators. Peptide-based modulators of nucleic acid ligands represent an alternative molecular class of modulators to oligonucleotides or their analogues. This class of modulators are particularly useful if sufficiently active oligonucleotide modulators of a target nucleic acid ligand cannot be isolated due to the lack of sufficient single-stranded regions to promote nucleation between the target and the oligonucleotide modulator. In addition, peptide modulators provide different bioavailabilities and pharmacokinetics than oligonucleotide modulators. In one exemplary embodiment the modulator is a protamine (Oney et al., 2009, Nat, Med. 15:1224-1228). Protamines are soluble in water, are not coagulated by heat, and comprise arginine, alanine and serine (most also contain proline and valine and many contain glycine and isoleucine). Modulators also include protamine variants (see e.g., Wakefield et al, J. Surg. Res. 63:280 (1996)) and modified forms of protamine, including those described in U.S. Publication No, 20040121443. Other modulators include protamine fragments, such as those described in U.S. Pat. No. 6,624,141 and U.S. Publication No. 20050101532. Modulators also include, generally, peptides that modulate the activity of heparin, other glycosaminoglycans or proteoglycans (see, for example, U.S. Pat. No. 5,919,761). In one exemplary embodiment, modulators are peptides that contain cationic-NH groups permitting stabilizing charge-charge interactions such as poly-L-lysine and poly-L-ornithine.

Several strategies to isolate peptides capable of binding to and thereby modulating the activity of a target nucleic acid ligand are available. For example, encoded peptide combinatorial libraries immobilized on beads have been described, and have been demonstrated to contain peptides able to bind viral RNA sequences and disrupt the interaction between the viral RNA and a viral regulatory protein that specifically binds said RNA (Hwang et al. Proc. Natl. Acad., Sci USA, 1999, 96:12997). Using such libraries, modulators of nucleic acid ligands can be isolated by appending a label to the target nucleic acid ligand and incubating together the labeled-target and bead-immobilized peptide library under conditions in which binding between some members of the library and the nucleic acid are favored. The binding of the nucleic acid ligand to the specific peptide on a given bead causes the bead to be “colored” by the label on the nucleic acid ligand, and thus enable the identification of peptides able to bind the target by simple isolation of the bead. The direct interaction between peptides isolated by such screening methods and the target nucleic acid ligand can be confirmed and quantified using any number of the binding assays described to identify modulators of nucleic acid ligands. The ability of said peptides to modulate the activity of the target nucleic acid ligand can be confirmed by appropriate bioassays.

In some embodiments, a modulator is a protein. For example, in certain embodiments, a nucleic acid ligand is linked to a biotin molecule. In those instances, a streptavadin or avidin is administered to bind to and reverse the effects of the ligand (see Savi et. al. J. Thrombosis and Haemostasis, 6: 1697-1706). Avidin is a tetrameric protein produced in the oviducts of birds, reptiles and amphibians which is deposited in the whites of their eggs. Streptavidin is a tetrameric protein purified from the bacterium Streptomyces avidinii. The tetrameric protein contains four identical subunits (homotetramer) each of which can bind to biotin (Vitamin B7, vitamin H) with a high degree of affinity and specificity. A protein modulator of a CLEC-2 nucleic acid ligand may be a soluble portion of the CLEC-2 domain that is bound by the CLEC-2 nucleic acid ligand. For example, the ECD or fragment thereof of the CLEC-2 polypeptide may compete with the CLEC-2 nucleic acid ligand for binding to cell-bound or native CLEC-2 to reverse binding of the CLEC-2 nucleic acid ligand to the native CLEC-2 molecule.

In an additional embodiment, the modulators are oligosaccharide based modulators. Oligosaccharides can interact with nucleic acids. For example, the antibiotic aminoglycosides are products of Streptomyces species and interact specifically with a diverse array of RNA molecules such as various ribozymes, RNA components of ribosomes, and HIV-1's TAR and RRE, sequences. Thus oligosaccharides can bind to nucleic acids and can be used to modulate the activity of nucleic acid ligands.

In another embodiment, the modulator is a small molecule based modulator. A small molecule that intercalates between the ligand and the target or otherwise disrupts or modifies the binding between the ligand and target can also be used as the therapeutic regulator. Such small molecules can be identified by screening candidates in an assay that measures binding changes between the ligand and the target with and without the small molecule, or by using an in vivo or in vitro assay that measures the difference in biological effect of the ligand for the target with and without the small molecule. Once a small molecule is identified that exhibits the desired affect, techniques such as combinatorial approaches can be used to optimize the chemical structure for the desired regulatory effect.

In a further exemplary embodiment, the modulator is a nucleic acid binding polymer, lipid, nanoparticle or microsphere. In further non-limiting examples, the modulator can be selected from the group consisting of: 1,2-dioleoyl-sn-glycero-3-ethylphosphocholine (EDOPC); dilauroylethylphosphatidylcholine (EDLPC); EDLPC/EDOPC; pyridinium surfactants; dioleoylphosphatidyl-ethanolamine (DOPE); (±)—N-(3-aminopropyl)-N,N-dimethyl-2,3-bis(dodecyloxy)-1-propanaminium bromide (GAP-DLRIE) plus the neutral co-lipid dioleoylphosphatidylethanolamine (DOPE) (GAP-DLRIE/DOPE); (±)-N,N-dimethyl-N4-[2-(spermine carboxamido)ethyl]-2,3-bis(dioeyloxy-1-propaniminium petahydrochloride (DOSPA); dilauroylethylphosphatidylcholine (EDLPC); Ethyldimyristoyl phosphatidylcholine (EDMPC); (±)-N,N,N-trimethyl-2,3-bis(z-octadec-9-ene-oyloxy)-1-propanaminium chloride (DOTAP); (±)-N-2-(2-hydroxyethyl)-N,N-dimethyl-2,3-bis(tetradecyloxy)-1-propanaminium bromide (DMRIE); (±)-N,N,N-trimethyl-2,3-bis(z-octadec-9-enyloxy)-1-propanaminium chloride (DOTMA); 5-carboxyspermylglycine dioctadecyl-amide (DOGS); dipalmitoylphosphatidylethanolamine 5-carboxyspermylamide (DPPES); 1,3 dioleoyloxy-2-(6-carboxyspermyl)-propyl-amid (DOSPER); tetramethyltetrapalmitoyl spermine (TMTPS); (tetramethyltetraoleyl spermine (TMTOS); tetramethyltetralauryl spermine (TMTLS); tetramethyltetramyristyl spermine (TMTMS); tetramethyldioleyl spermine (TMDOS); diphytanoylphosphatidyl-ethanolamine (DPhPE); and (±)-N-(3-aminopropyl)-N,N-dimethyl-2,3-bis(dodecyloxy)-1-propanaminium bromide (GAP-DLRIE).

alcohol); acrylic or methacrylic polymer; Newkome dendrimer; polyphenylene; dimethyldioctadecylammonium bromide (DAB); cetyltrimethylammonium bromide (CTAB); albumin; acid-treated gelatin; polylysine; polyornithine; polyarginine; DEAE-cellulose; DEAF-dextran; and poly(N,N-dimethylaminoethylmethacrylate); and polypropylamine (POPAM).

In one embodiment, the modulator is selected from chitosan and chitasan derivatives. Chitosan derivatives include water soluble chitosan nanoparticles (such as described in U.S. Pat. No. 6,475,995; US Patent Application No. 2006/0013885; Limpeanchob et al, (2006) Efficacy and Toxicity of Amphotericin B-Chitosan Nanoparticles; Nareusan University Journal 14(2):27-34). Given the polycationic nature of the chitosan polymer (essentially a very large polyamine polymer composed of repeating glucosamine monomers), chitosan may be used to aggregate and/or encapsulate ligands into a polyelectrolyte complex in vivo following injection into a host. This is based in part on interactions of the primary amines found on chitosan and the phosphodiester backbone of the ligand.

In other embodiments, the modulator is selected from the group consisting 1,5-dimethyl-1,5-diazaundecamethylene polymethobromide; polyoxyethylene/polyoxypropylene Hock copolymers; poly-L-lysine; polyamidoamine (PAMAM); β-cyclodextrin-containing polycation (CDP); β-cyclodextrin-containing polycation (imidazole-containing variant) (CDP-Im); polyphosphoramidate polymer (8 kDa, 30 kDa) (PPA-DPA 8k, PPA-DPA 30k); polybrene; spermine; PEG-block-PLL-dendrimers; polyethylenimine (PEI); mannose-PEI; transferin-PEI; linear-PEI (IPEI); gelatin; methacrylate/methacrylamide; poly(beta-amino esters); polyelectrolyte complexes (PEC); poly(vinalyamine) (INA); Collagen; polypropylene imine (PPI); polyallylamine; polyvinylpyridine; aminoacetalized poly(vinyl

In certain embodiments, the primary amines on the chitosan polymer can be substantially modified to alter the water solubility and charge state. Chitosan derivatives include trimethyl chitosan chloride (TMC), which can be synthesized at different degrees of quaternization; mono-carboxymethylated chitosan (MCC) which is a polyampholytic polymer; glutaraldehyde cross-linked derivative (CSGA); thiolated chitosan (Lee, et al. (2007) Pharm. Res. 24:157-67); glycol chitosan (GC), a chitosan derivative conjugated with ethylene glycol (Lee, et al. (2007) Int J Pharm.); [N-(2-carboxybenzyl)chitosan (CBCS) (Lin, et al. (2007) Carbohydr Res. 342(1):87-95); a beta-cyclodextrin-chitosan polymer (Venter, et al. (2006) Int J Pharm. 313(1-2):36-42); O-carboxymethylchitosan; N,O-carboxymethyl chitosan; or a chitosan chemically modified by introducing xanthate group onto its backbone.

In one embodiment, empty chitosan nanoparticles are generated and used as modulators. Chitosan or chitosan derivatives of molecular weight range of 10,000 Da to >1,000,000 Da may be used. In certain embodiments, the chitosan is of 500,000 Da or less. In certain embodiments, the chitosan is of 100,000 Da or less. In some embodiments, the compound is between 10,000 and 100,000 Da, between 10,000 and 90,000, between 10,000 and 80,000, between 20,000 and 70,0000, between 30,000 and 70,000, about 30,000, about 40,000, about 50,000 or about 60,000 Da.

In some embodiments, chitosan polymers containing different degrees of deacetylated primary amines are used. In these embodiments, the different degrees of deacetylation alters the charge state of the polymer and thereby the binding properties of the polymer. Upon contact of the chitosan nanoparticle with ligands in the host, ligands may bind with and become trapped on the nanoparticle surface, or enter the nanoparticle and become encapsulated by ionic interactions.

In another embodiment, the modulator is a polyphosphate polymer microsphere. In certain embodiments, the modulator is a derivative of such a microsphere such as poly(L-lactide-co-ethyl-phosphite) or P(LAEG-EOP) and others, as described in U.S. Pat. No. 6,548,302. Such polymers can be produced to contain a variety of functional groups as part of the polymeric backbone. In one example, the polymers may contain quaternary amines with a positive charge at physiologic pH, such that they can complex or encapsulate one or more nucleic acids upon contact. In certain embodiments, the polymers do not contain positive charges.

In certain embodiments, a modulator is a cationic molecule. In certain embodiments, the ligand forms a guanine quartet (O-quartet or G-quadruplex) structure. These structures are bound by cationic molecules. In certain embodiments, the molecules are metal chelating molecules. In some embodiments, the modulator is a porphyrin. In some embodiments, the compound is TMPyP4. See Joachimi, et. al. JACS 2007, 129, 3036-3037 and Toro, et. al. Analytical Biochemistry 2008 Aug. 1, 379 (1) 8-15.

Modulators can be identified in general, through binding assays, molecular modeling, or in vivo or in vitro assays that measure the modification of biological function. In one embodiment, the binding of a modulator to a nucleic acid is determined by a gel shift assay. In another embodiment, the binding of a modulator to a nucleic acid ligand is determined by a BIACORE assay.

Standard binding assays can be used to identify and select modulators. Non-limiting examples are gel shift assays and BIACORE assays. That is, test modulators can be contacted with the nucleic acid ligands to be targeted under test conditions or typical physiological conditions and a determination made as to whether the test modulator in fact binds the ligand. Test modulators that are found to bind the nucleic acid ligand can then be analyzed in an appropriate bioassay (which will vary depending on the ligand and its target molecule, for example coagulation tests) to determine if the test modulator can affect the biological effect caused by the ligand on its target molecule.

The Get-Shift assay is a well-known technique used to assess binding capability. For example, a nucleic acid ligand to CLEC-2 is first incubated with CLEC-2 protein or fragment thereof, or a mixture containing the CLEC-2 protein or fragment, and then separated on a gel by electrophoresis Upon binding of the ligand to the protein, the complex will be larger in size and its migration will therefore be retarded relative to that of the free ligand which can be applied to a control lane in the gel in the absence of CLEC-2 protein. The ligand can be labeled, for example, by a radioactive or nonradioactive moiety, to allow detective of the ligand CLEC-2 complex within the gel. When using the Gel-Shift assay to screen for ligands having CLEC-2-binding activity, the complex can then be extracted from the gel and the isolated ligand analyzed to identify ligands having the desired CLEC-2 binding activity.

Gel shift assays can also be used to screen modulators for binding nucleic acid ligands to CLEC-2, as association of the modulator with the nucleic acid ligand retards the mobility of the nucleic acid ligand relative to that of the free ligand (see, for example, Rusconi et al., 2002, Nature, 419:90-94.).

Additionally, modulators can be added to such an assay format and screened for their ability to block association of a CLEC-2 nucleic acid ligand with CLEC-2. For example, the CLEC-2-ligand mixture can be incubated in the presence of increasing amounts of potential modulator. A modulator with the desired activity will specifically reduce formation of the CLEC-2-ligand complex as detected by the Gel-Shift assay.

BIACORE technology is known to the skilled artisan as a reliable and valuable tool for identifying and analyzing macromolecular interactions, include polypeptide-nucleic acid interactions. Accordingly, one could use this technology to screen for or to identify nucleic acid aptamers or ligands which specifically bind the CLEC-2 protein or fragment thereof. The BIACORE technology measures binding events on a sensor chip surface, so that an interactant attached to the surface determines the specificity of the analysis. In other words, the CLEC-2 protein or fragment could be attached to the sensor chip surface via, for example, a histidine tag. The bound CLEC-2 proteins are then exposed to a solution containing the potential ligand molecules. Binding of the nucleic acid ligand to the CLEC-2 protein gives an immediate change in the surface plasmon resonance (SPR) signal. The signal is directly proportional to the mass of molecules that bind to the surface.

As described for the gel-shift assay, the BIACORE could be used to identify or analyze modulators of the CLEC-2 ligands. Again, the reaction mixture to which the chip-bound CLEC-2 protein is exposed can contain both a known CLEC-2 ligand with increasing amounts of modulator and the effects determined by standard BIACORE analysis of the resultant interaction between CLEC-2 and its ligand.

There are a number of other assays that can determine whether an oligonucleotide or analogue thereof, peptide, polypeptide, oligosaccharide or small molecule can bind to the ligand in a manner such that the interaction with the target is modified. For example, electrophoretic mobility shift assays (EMSAs), titration calorimetry, scintillation proximity assays, sedimentation equilibrium assays using analytical ultracentrifugation (see for eg. www.cores.utah.edu/interaction), fluorescence polarization assays, fluorescence anisotropy assays, fluorescence intensity assays, fluorescence resonance energy transfer (FRET) assays, nitrocellulose filter binding assays, ELISAs, ELONAs (see, for example, U.S. Pat. No. 5,789,163), RIAs, or equilibrium dialysis assays can be used to evaluate the ability of an agent to bind to a nucleic acid ligand. Direct assays in which the interaction between the agent and the nucleic acid ligand is directly determined can be performed, or competition or displacement assays in which the ability of the agent to displace the ligand from its target can be performed (for example, see Green, Bell and Janjic, Biotechniques 30(5), 2001, p 1094 and U.S. Pat. No. 6,306,598). Once a candidate modulating agent is identified, its ability to modulate the activity of a nucleic acid ligand for its target can be confirmed in a bioassay. Alternatively, if an agent is identified that can modulate the interaction of a ligand with its target, such binding assays can be used to verify that the agent is interacting directly with the ligand and can measure the affinity of said interaction.

In another embodiment, mass spectrometry can be used for the identification of a modulator that binds to a nucleic acid ligand, the site(s) of interaction between the modulator and the nucleic acid ligand, and the relative binding affinity of agents for the ligand (see for example U.S. Pat. No. 6,329,146). Such mass spectral methods can also be used for screening chemical mixtures or libraries, especially combinatorial libraries, for individual compounds that bind to a selected target ligand that can be used in as modulators of the ligand. Furthermore, mass spectral techniques can be used to screen multiple target nucleic acid ligand simultaneously against, e.g. a combinatorial library of compounds. Moreover, mass spectral techniques can be used to identify interaction between a plurality of molecular species, especially “small” molecules and a molecular interaction site on a target ligand.

In another embodiment, in vivo or in vitro assays that evaluate the effectiveness of a modulator in modifying the interaction between a nucleic acid ligand and a target which are specific for the disorder being treated can be used for the identification of a modulator that binds to a nucleic acid ligand. There are ample standard assays for biological properties that are well known and can be used. Examples of biological assays are provided in the patents cited in this application that describe certain nucleic acid ligands for specific applications.

In one embodiment, the modulator has the ability to substantially bind to a nucleic acid ligand in solution at modulator concentrations of less than ten (10.0) micromolar (μM), one (1.0) micromolar (μM), preferably less than 0.1 μM, and more preferably less than 0.01 μM. By “substantially” is meant that at least a 50 percent reduction in target biological activity is observed by modulation in the presence of the target, and at 50% reduction is referred to herein as an IC₅₀ value.

### Optimizing Ligands and Modulators

In order for a ligand to be suitable for use as a therapeutic, the ligand is preferably inexpensive to synthesize, safe for use in a host, and stable in vivo. Wild-type RNA and DNA oligonucleotides are typically not stable in vivo because of their susceptibility to degradation by nucleases. Resistance to nuclease degradation can be greatly increased by the incorporation of modifying groups at the 2′-position.

2′-fluoro or amino groups may be incorporated into oligonucleotide pools from which ligands have been subsequently selected. In the present disclosure, 2′-fluoropyrimidines were used in an in vitro transcription reaction to generate an initial oligonucleotide pool for ligand selection (see Example 1).

After initial identification of the ligands (via e.g., SELEX) and the modulators (e.g., design based on sequence complementarity), the ligands and modulators can be modified or engineered to improve their desired structure, function and/or stability by a variety of means. These include, but are not limited to, substituting particular sugar residues, changing the composition and size of particular regions and/or structures in the ligand, and designing ligands that can be more effectively regulated by a modulator.

The design and optimization of a nucleic acid ligand involves an appreciation for the secondary structure of the ligand as well as the relationship between the secondary structure and the modulator control. Unlike conventional methods of modifying nucleic acids, the design of the ligands to the CLEC-2 protein may include consideration of the impact of changes to the ligand on the design of potential modulators, if a ligand is modified by truncation, for example, the corresponding modulator should be designed to control the truncated ligand.

The secondary structure of ligands identified through the SELEX process can be predicted by various methods known to persons having ordinary skill in the art. For example, each sequence may be analyzed using a software program such as Mfold (mfold.bioinfo.rpi.edu; see also Zuker, 2003, Nucleic Acids Res. 31:3406-3415 and Mathews, et al., 1999, J. Mol, Biol. 288:911-940). Subsequently, comparative sequence analysis of the various selected sequences can be used to align the sequences based upon conserved consensus secondary structural elements to arrive at a predicted secondary consensus structure for CLEC-2 ligands.

CLEC-2 nucleic ligands as disclosed herein can be modified by varying overall ligand length as well as the lengths of the stem and loop structures. For example, ligand truncations may be generated in which a portion of the 5′ and/or 3′ end of a ligand is deleted from the ligand selected in the SELEX process. To determine the extent of truncations which are tolerated by a ligand, one method used can be to heat anneal an oligonucleotide (e.g. a DNA oligonucleotide) complementary to a 5′ or 3′ terminal region of the ligand, then compare binding of the ligand with and without the annealed oligonucleotide. If no significant binding difference is observed between the ligand with and the ligand without the annealed oligonucleotide, this suggests that the annealed portion of the ligand is dispensable for binding of the ligand to the target protein. This method can be performed using oligonucleotides which anneal to various lengths of the 5′ or 3′ ends of the ligand to determine 5′ and 3′ boundaries which provide a fully functional ligand.

In another embodiment, the design includes decreasing the size of the ligand. In another embodiment, the size of the modulator is changed in relation to the size of the ligand. In yet another embodiment, guanine strings are reduced to less than tour guanine, or less than three guanine, or less than two guanine or no guanines. However, the joint effect of these changes must meet the challenge of creating a ligand that provides adequate activity but is easily neutralized by the modulator.

For targeting of a modulator, an improved ligand can also be modified so as to include a single-stranded tail (3′ or 5′) in order to promote association with an oligonucleotide modulator. Suitable tails can comprise 1 nt to 20 nt, preferably, 1 nt to 10 nt, 1 nt to 5 nt or 3 nt to 5 nt, wherein the nucleotide at each position within the tail may be A, C, G, T, or U. It is readily understood that such tails may included modified nucleotides as described in more detail below.

Tailed ligands can be tested in binding and bioassays e.g., as described below) to verify that addition of the single-stranded tail does not disrupt the active structure of the ligand. A series of oligonucleotides (for example, 2′-O-methyl oligonucleotides) that can form, for example, 1, 3 or 5 base-pairs with the tail sequence can be designed and tested for their ability to associate with the tailed ligand alone, as well as their ability to increase the rate of dissociation of the ligand from, or association of the ligand with, its target molecule. Scrambled sequence controls can be employed to verify that the effects are due to duplex formation and not non-specific effects.

CLEC-2 ligands may also be designed to have a suicide position, which allows more effective regulation by paired modulators. Upon binding of the ligand by the modulator, the suicide position becomes single stranded and labile, thereby facilitating cleavage of the ligand by enzymes naturally present in the blood, such as blood or liver endonucleases. This provides a means for effective and substantially immediate elimination of the active ligand from circulation.

### Chemical Modifications

One problem encountered in the therapeutic use of nucleic acids is that oligonucleotides in their phosphodiester form may degrade in body fluids by intracellular and extracellular enzymes such as endonucleases and exonucleases before the entire effect is manifest. Certain chemical modifications of the nucleic acid ligand can increase the in vivo stability of the nucleic acid ligand or to enhance or to mediate the delivery of the nucleic acid ligand. Additionally, certain chemical modifications can increase the affinity of the nucleic acid ligand for its target, by stabilizing or promoting the formation of required structural elements within the nucleic acid ligand or providing additional molecular interactions with the target molecule.

Modifications of the ligands can include, but are not limited to, those which provide chemical groups that incorporate additional charge, polarizability, hydrophobicity, hydrogen bonding, electrostatic interactions, and functionality to the nucleic acid ligand bases or to the ligand as a whole. Modification may be at the base moiety, sugar moiety, or phosphate backbone, for example, to improve properties such as stability of the molecule and affinity for the intended target. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil, backbone modifications, phosphorothioate or alkyl phosphate modifications, methylations, unusual base-pairing combinations such as the isobases isocytidine and isoguanidine and the like. Modifications can also include 3 and 5′ modifications such as capping.

The SELEX method encompasses the identification of high-affinity nucleic acid ligands containing modified nucleotides conferring improved characteristics on the ligand, such as improved in vivo stability or improved delivery characteristics. Examples of such modifications include chemical substitutions at the ribose and/or phosphate and/or base positions. SELEX-identified nucleic acid ligands containing modified nucleotides are described in U.S. Pat. No. 5,660,985 that describes oligonucleotides containing nucleotide derivatives chemically modified at the 5- and 2′-positions of pyrimidines. U.S. Pat. No. 5,580,737 describes specific nucleic acid ligands containing one or more nucleotides modified with 2′-amino (2—NH2), 2′-fluoro (2′-F), and/or 2′-O-methyl (2′-OMe).

The SELEX method encompasses combining selected oligonucleotides with other selected oligonucleotides and non-oligonucleotide functional units as described in U.S. Pat. Nos. 5,637,459 and 5,683,867. U.S. Pat. No. 5,637,459 describes highly specific nucleic acid ligands containing one or more nucleotides modified with 2′-amino (2′—NH 2), 2′-fluoro (2′-F), and/or 2′-O-methyl (2′-OMe). The SELEX method further encompasses combining selected nucleic acid ligands with lipophilic or Non-Immunogenic, High Molecular Weight compounds in a diagnostic or therapeutic complex as described in U.S. Pat. No. 6,011,020.

Where the nucleic acid ligands are derived by the SELEX method, the modifications can be pre- or post-SELEX modifications. Pre-SELEX modifications can yield ligands with both specificity for its target and improved in vivo stability. Post-SELEX modifications made to 2′-hydroxyl (2′-OH) nucleic acid ligands can result in improved in vivo stability without adversely affecting the binding capacity of the nucleic acid ligands. In one embodiment, the modifications of the ligand include a 3′-3′ inverted phosphodiester linkage at the 3′ end of the molecule, and 2′ fluoro (2′-F), 2′ amino (2′-NH2), 2′ deoxy, and/or 2′O methyl (2′-OMe) modification of some or all of the nucleotides.

The ligands described herein were initially generated via SELEX using libraries of transcripts in which the C and U residues were 2′-fluoro substituted and the A and G residues were 2′-OH.

In certain embodiments, the nucleic acids making up the ligand include modified sugars and/or modified bases. In certain embodiments, the modifications include stabilizing modifications such as 2′-stabilizing modifications. In one embodiment, 2′-stabilizing modifications can include 2′-fluoro modifications on the sugar ring.

In one embodiment, the design includes decreasing the T-hydroxyl content of the ligand or the modulator, or both. In another embodiment, the design includes decreasing the 2′-fluoro content of the ligand or the modulator, or both. In another embodiment, the design includes increasing the 2′-O-methyl content of the ligand or the modulator, or both.

In pharmaceutical compositions the ligands can be provided in forms, such as salt forms that improve solubility or bioavailability. Suitable salts include inorganic cations such as sodium and potassium.

Any of the oligonucleotides of the disclosure can be synthesized by standard methods known in the art, e.g. by use of an automated DNA synthesizer (such as are commercially available from, for example, Biosearch, Applied Biosystems).

Ligands and modulators are described herein using abbreviations readily understood by a skilled artisan and noted as follows: “rA” is 2′OH A or adenosine; “A” is a 2′-deoxy A or 2′-deoxyadenosine; “mA” is 2′-O-methyl A or 2′-methoxy-2′-deoxyadenine; “rG” is 2′-OH G or guanosine; “G” is a 2′-deoxy G or 2′-deoxyguanosine; “mG” is 2′-O-methyl G or 2′-methoxy-2′ deoxyguanosine; “fC” is 2′-fluoro C or 2′-fluoro-2′ deoxycytidine; “Inc” is 2′-O-methyl C or methoxy-2′-deoxycytidine; “fU” is 2′-fluoro U or 2′-fluoro-uridine; “mU” is 2′-O-methyl U or 2′-methoxy-uridine; and “iT” is inverted 2′H T.

### Coupling to a Carrier

The ligands as described herein can also include modifications that improve bioavailability or stability. Such modifications can include conjugation to a carrier molecule which may include, but is not limited to a hydrophilic or hydrophobic moiety.

### Methods to Treat CLEC-2 Mediated Disorders

In one embodiment, provided is a method for treating a CLEC-2 mediated disorder comprising administering to a host in need thereof a therapeutically effective amount of an ligand or a pharmaceutically acceptable salt thereof as described herein.

CLEC-2 was first identified as a mediator of platelet aggregation (for a review, see Suzuki—Inoue et al, 2011, J. Thromb Haemostasis, 9(S1):44-55). Subsequently, CLEC-2 was shown to be involved in thrombus formation and stabilization. Accordingly, CLEC-2 ligands which are able to inhibit the function or activity of CLEC-2 can be used for therapeutic intervention of a variety of platelet-mediated disorders. In one embodiment, the CLEC-2 mediated disorder comprises avascular disorder. In another embodiment, the vascular disorder is selected from the group consisting of acute coronary syndromes, thrombosis, thromboembolism, thrombocytopenia, peripheral vascular disease, and transient ischemic attack. The compositions described herein are also useful for treatment of a cerebrovascular disorder, including but not limited to, transient ischemic attack, ischemic stroke, and embolism.

In one embodiment, the CLEC-2 mediated disorder is a diabetes-related disorder. In one embodiment, the diabetes-related disorder is selected from the group consisting of diabetic retinopathy, diabetic vasculopathy, atherosclerosis, ischemic stroke, peripheral vascular disease, acute renal injury and chronic renal failure.

In one embodiment, the CLEC-2 mediated disorder comprises a platelet-mediated inflammatory disorder. In another embodiment, the platelet-mediated inflammatory disorder selected from the group consisting of arthritis, rheumatoid arthritis, psoriatic arthritis, reactive arthritis, inflammatory bowed disease, ankylosing spondylitis, and scleroderma.

In one embodiment, the CLEC-2 mediated disorder is a cancer. It has long been suggested that platelets are involved in cancer metastasis and/or progression (Nash et al., 202, Lancet Oncol, 3:425-430). Platelet aggregates surrounding tumor cells may protect them from shear stress and NK cells in the blood, thereby facilitating formation of tumor cell nests. Moreover, since the discovery of podoplanin as an endogenous CLEC-2 ligand, experimental data have suggested that CLEC-2/podoplanin may be a viable target for anti-metastatic drugs. For example, an anti-podoplanin blocking antibody significantly inhibited the number of metastatic lung nodules consisting of tumor cells expressing podoplanin (Kato et al., 2008, Cancer Sci, 99:54-61).

The CLEC-2 ligands described herein, which are shown to inhibit CLEC-2-mediated platelet aggregation, have therapeutic use in the treatment and inhibition of tumor metastasis. In one embodiment, the cancer is selected from the group consisting of lung cancer, breast cancer, prostate cancer, testicular cancer, pancreatic cancer, brain cancer, bone cancer and liver cancer. However, it is understood that CLEC-2 ligands described herein may be useful in treating or inhibiting any cancer which has metastasized or is considered by the skilled artisan to be likely to metastasize.

In one embodiment, the CLEC-2 ligand inhibits initiation of platelet activation. In other embodiments, the CLEC-2 ligand inhibits platelet activation and the resultant platelet pro-inflammatory response. In other embodiments, the CLEC-2 ligand inhibits platelet adhesion. In other embodiments, the CLEC-2 ligand inhibits platelet aggregation. In yet a further embodiment, the CLEC-2 ligand inhibits thrombin generation.

In certain embodiments a method of treating or preventing formation of a vascular event, in particular a thrombotic or thromboembolitic event is provided including administering a CLEC-2 nucleic acid ligand as described herein to a host in need thereof.

In one embodiment, the CLEC-2 nucleic acid ligand is provided for extended periods of time. In this instance, a CLEC-2 ligand modulator may only be used in emergency situations, for example, if treatment leads to hemorrhage, including intracranial or gastrointestinal hemorrhage, in another embodiment, the modulator is administered when emergency surgery is required for patients who have received CLEC-2 nucleic acid ligand treatment. In another embodiment, the modulator is administered to control the concentration of the CLEC-2 nucleic acid ligand and thereby the duration and intensity of treatment. In another embodiment, the CLEC-2 nucleic acid ligand is provided as a platelet anesthetic during a cardiopulmonary bypass procedure. In another embodiment, the CLEC-2 nucleic acid ligand is administered to provide a period of transition off of or on to oral anti-platelet medications, and the modulator is used to reverse the CLEC-2 nucleic acid ligand once therapeutic levels of the oral anti-platelet agent are established.

The methods as described herein may be used in combination with another therapy.

In one embodiment, the host is preparing to undergo or undergoing a surgical intervention, or has undergone a surgical intervention that puts the host at risk of an occlusive thrombotic event. In other embodiments, the host has received a vessel graft to enable hemodialysis, which is at risk of occluding due to interactions between the vessel and platelets.

In one embodiment the therapy includes treating a host with a therapeutically effective amount of an anti-cancer or an anti-thrombotic agent.

In one embodiment, the therapy includes treating a host with a therapeutically effective amount of an anti-HIV agent selected from the group consisting of HIV antiviral agents, immunomodulators, and anti-infective agents.

### Administration and Pharmaceutical Composition

The CLEC-2 nucleic acid ligands or CLEC-2 ligand modulators taught herein can be formulated into pharmaceutical compositions that can include, but are not limited to, a pharmaceutically acceptable carrier, diluent or excipient. The precise nature of the composition will depend, at least in part, on the nature of the ligand and/or modulator, including any stabilizing modifications, and the route of administration. Compositions containing the modulator can be designed for administration to a host who has been given a CLEC-2 nucleic acid ligand to allow modulation of the activity of the ligand, and thus regulate anti-platelet activity of the administered CLEC-2 nucleic acid ligand.

The design and preparation of pharmaceutical or pharmacological compositions will be known to those of skill in the art in light of the present disclosure. Typically, such compositions can be prepared as injectables, either as liquid solutions or suspensions; solid forms suitable for solution in, or suspension in, liquid prior to injection; as tablets or other solids for oral administration; as time release capsules, as liquids for oral administration; as elixirs, syrups, suppositories, gels, or in any other form used in the art, including eye drops, creams, lotions, salves, inhalants and the like. The use of sterile formulations, such as saline-based washes, by surgeons, physicians or health care workers to treat a particular area in the operation field may also be particularly useful. Compositions can also be formulated for delivery via microdevice, microparticle or sponge. Compositions can also be coated on implanted medical devices, such as stents, for local delivery.

Pharmaceutically useful compositions comprising a CLEC-2 nucleic acid ligand or CLEC-2 ligand modulator can be formulated at least in part by the admixture of a pharmaceutically acceptable carrier. Examples of such carriers and methods of formulation can be found in Remington: The Science and Practice of Pharmacy, 20th edition (Lippincott Williams & Wilkins, 2000) and Ansel et al., Pharmaceutical Dosage Forms and Drug Delivery Systems, 6th Ed, (Media, Pa.: Williams & Wilkins, 1995).

Suitable pharmaceutical excipients include stabilizers, antioxidants, osmolality adjusting agents, buffers, and pH adjusting agents, including but not limited to phosphate-buffered saline, Suitable additives include physiologically biocompatible buffers (e.g., tromethamine hydrochloride), additions of chelants (such as, for example, EDTA, DTPA or DTPA-bisamide) or calcium chelate complexes (as for example calcium DTPA, CaNaDTPA-bisamide), or, optionally, additions of calcium or sodium salts (for example, sodium chloride, calcium chloride, calcium ascorbate, calcium gluconate or calcium lactate). Pharmaceutical compositions can be packaged for use in liquid form, or can be lyophilized.

To form a pharmaceutically acceptable composition suitable for effective administration, such compositions will contain an effective amount of the nucleic acid ligand or modulator. Such compositions can contain admixtures of more than one compound. The compositions typically contain about 0.1% weight percent (wt %) to about 50 wt %, about 1 wt % to about 25 wt %, or about 5 wt % to about 20 wt % of the active agent (ligand or modulator).

Pharmaceutical compositions for parenteral administration, including subcutaneous, intramuscular intravenous injections and infusions are provided herein. For parenteral administration, aseptic suspensions and solutions are desired. Isotonic preparations that generally contain suitable preservatives are employed when intravenous administration is desired. The pharmaceutical compositions may be sterilized and/or contain adjuvants, such as preserving, stabilizing, wetting or emulsifying agents, solution promoters, salts for regulating the osmotic pressure and/or buffers, Liquid, particularly injectable compositions can, for example, be prepared by dissolving, dispersing, etc. The active compound is dissolved in or mixed with a pharmaceutically pure solvent such as, for example, water, buffered water, saline, 0.4% saline, 0.3% glycine, hyaluronic acid, aqueous dextrose, glycerol, ethanol, and the like, to thereby form the injectable solution or suspension. Additionally, solid forms suitable for dissolving in liquid prior to injection can be formulated.

To aid dissolution of an agent into an aqueous environment, a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride, Nonionic detergents that could be included in the formulation as surfactants include, but are not limited to, lauromacrogol 400, polyoxyl 40 stearate, polyoxyethylene hydrogenated castor oil 10, 50 and 60, glycerol monostearate, polysorbate 20, 40, 60, 65 and 80, sucrose fatty acid ester, methyl cellulose, carboxymethyl cellulose and any of the pluronic detergents such as Pluronic F68 and/or Pluronic F127 (e.g., see Strappe et al. Eur. J. of Pharm. Biopharm., 2005, 61:126-133). Surfactants could be present in the formulation of a protein or derivative either alone or as a mixture in different ratios.

For oral administration in the form of a tablet or capsule, the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture, Suitable binders include without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes and the like, Lubricants used in these dosage forms include, without limitation, sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

For liquid forms used in oral administration, the active drug component can be combined in suitably flavored suspending or dispersing agents such as the synthetic and natural gums, for example, tragacanth, acacia, methyl-cellulose and the like. Other dispersing agents that can be employed include glycerin and the like.

Topical preparations containing the active drug component can be admixed with a variety of carrier materials well known in the art, such as, e.g., alcohols, aloe vera gel, allantoin, glycerine, vitamin A and E oils, mineral oil, PPG2 myristyl ether propionate, and the like, to form, e.g., alcoholic solutions, topical cleansers, cleansing creams, skin gels, skin lotions, and shampoos in cream or gel formulations.

The ligands can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines. Active agents administered directly (e.g., alone) or in a liposomal formulation are described, for example, in U.S. Pat. No. 6,147,204.

The ligand can also be coupled with soluble polymers as targetable drug carriers. Such polymers can include polyvinyl-pyrrolidone, pyran copolymer, polyhydroxypropylmethacryl-amide-phenol, polyhydroxy-ethylaspartamidephenol, or polyethyl-eneoxidepolylysine substituted with palmitoyl residues. Furthermore, the ligands can be coupled (preferably via a covalent linkage) to a class of biodegradable polymers useful in achieving controlled release of a drug, for example, polyethylene glycol (PEG), polytactic acid, polyepsilon caprolactone, polyoxazolines, polyhydroxy butyric acid, polyorthoesters, polyacetals, polydihydro-pyrans, polycyanoacrylates and cross-linked or amphipathic block copolymers of hydrogels. Cholesterol and similar molecules can be linked to the nucleic acid ligands to increase and prolong bioavailability.

Lipophilic compounds and non-immunogenic high molecular weight compounds with which the modulators of the disclosure can be formulated for use can be prepared by any of the various techniques presently known in the art or subsequently developed. Typically, they are prepared from a phospholipid, for example, distearoyl phosphatidylcholine, and may include other materials such as neutral lipids, for example, cholesterol, and also surface modifiers such as positively charged (e.g., sterylamine or aminomannose or aminomannitol derivatives of cholesterol) or negatively charged (e.g., diacetyl phosphate, phosphatidyl glycerol) compounds. Multilamellar liposomes can be formed by the conventional technique, that is, by depositing a selected lipid on the inside wall of a suitable container or vessel by dissolving the lipid in an appropriate solvent, and then evaporating the solvent to leave a thin film on the inside of the vessel or by spray drying. An aqueous phase is then added to the vessel with a swirling or vortexing motion which results in the formation of multilamellar liposome vesicles (MLVs). Unilamellar liposome vesicles (UVs) can then be formed by homogenization, sonication or extrusion (through filters) of MLVs. In addition, UVs can be formed by detergent removal techniques. In certain embodiments of this disclosure, the complex comprises a liposome with a targeting nucleic acid ligand(s) associated with the surface of the liposome and an encapsulated therapeutic or diagnostic agent. Preformed liposomes can be modified to associate with the nucleic acid ligands. For example, a cationic liposome associates through electrostatic interactions with the nucleic acid. Alternatively, a nucleic acid attached to a lipophilic compound, such as cholesterol, can be added to preformed liposomes whereby the cholesterol becomes associated with the liposomal membrane. Alternatively, the nucleic acid can be associated with the Liposome during the formulation of the liposome.

In another embodiment, a stent or medical device may be coated with a formulation comprising a CLEC-2 ligand or CLEC-2 modulator according to methods known to skilled artisans.

Therapeutic kits are also envisioned. The kits comprises the reagents, active agents, and materials that may be required to practice the above methods. The kits will generally contain, in suitable container means, a pharmaceutically acceptable formulation of a CLEC-2 ligand and/or a CLEC-2 ligand modulator. Where the kit comprises both a CLEC-2 ligand and a CLEC-2 modulator, the CLEC-2 modulator, in some embodiments, is a modulator which binds the CLEC-2 ligand in the kit. The kit may have a single container means, and/or it may have distinct container means for each compound.

### Methods for Administration

Modes of administration of the CLEC-2 ligands and/or CLEC-2 ligand modulators of the present disclosure to a host include, but are not limited to, parenteral (by injection or gradual infusion over time), intravenous, intradermal intra-articular, intra-synovial, intrathecal intra-arterial, intracardiac, intramuscular, subcutaneous, intraorbital, intracapsular, intraspinal, intrasternal, topical, transdermal patch, via rectal, buccal, vaginal or urethral suppository, peritoneal, percutaneous, nasal spray, surgical implant, internal surgical paint, infusion pump or via catheter. In one embodiment, the agent and carrier are administered in a slow release formulation such as an implant, bolus, microparticle, microsphere, nanoparticle or nanosphere. In one embodiment, the CLEC-2 nucleic acid ligand is delivered via subcutaneous injection or deposition including subcutaneous infusion (such as by osmotic pumps).

In one embodiment, the CLEC-2 nucleic acid ligand is delivered via subcutaneous administration and the modulator is delivered by subcutaneous or intravenous administration.

The therapeutic compositions comprising ligands and modulators of the present disclosure may be administered intravenously, such as by injection of a unit dose. The term “unit dose” when used in reference to a therapeutic composition refers to physically discrete units suitable as unitary dosage for the host, each unit containing a predetermined quantity of active material calculated to produce the desired therapeutic effect in association with the required diluent; i.e., carrier or vehicle.

Additionally, one approach for parenteral administration employs the implantation of a slow-release or sustained-released systems, which assures that a constant level of dosage is maintained.

Local administration, for example, to the interstitium of an affected joint, is also provided. Local administration can be achieved by injection, such as from a syringe or other article of manufacture containing a injection device such as a needle. The rate of administration from a syringe can be controlled by controlled pressure over desired period of time to distribute the contents of the syringe. In another example, local administration can be achieved by infusion, which can be facilitated by the use of a pump or other similar device.

Representative, non-limiting approaches for topical administration to a vascular tissue are also provided and include (1) coating or impregnating a blood vessel tissue with a gel comprising a nucleic acid ligand, for delivery in vivo, e.g., by implanting the coated or impregnated vessel in place of a damaged or diseased vessel tissue segment that was removed or by-passed; (2) delivery via a catheter to a vessel in which delivery is desired; (3) pumping a composition into a vessel that is to be implanted into a patient. Alternatively, the compounds can be introduced into cells by microinjection, or by liposome encapsulation.

Also provided is administration of the CLEC-2 ligands to a subject by coating medical devices such as stents with pharmaceutical compositions containing the ligand. Methods for coating to allow appropriate release and administration of the ligand are known to those having ordinary skill in the art.

Optimum dosing regimens for the compositions described herein can be readily established by one skilled in the art and can vary with the modulator, the patient and the effect sought. The effective amount can vary according to a variety of factors such as the individual's condition, weight, sex, age and amount of nucleic acid ligand administered. Other factors include the mode of administration.

The pharmaceutically effective dose depends on the type of disease, the composition used, the route of administration, the type of mammal being treated, the physical characteristics of the specific mammal under consideration, concurrent medication, and other factors that those skilled in the medical arts will recognize Generally, the compositions will be administered in dosages adjusted for body weight, e.g., dosages ranging from about 1 μg/kg body weight to about 100 mg/kg body weight. More typically, the dosages will range from about 0.1 mg/kg to about 20 mg/kg, and more typically from about 0.5 mg/kg to about 10 mg/kg, or about 1.0 to about 5.0 mg/kg, or about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg or about 10.0 mg/kg. Typically, the dose initially provides a plasma concentration of drug about 0.002 μg/ml to about 2000 μg/ml of drug, more typically from about 2.0 μg/ml to about 400 μg/ml, and more typically from about 10 μg/ml to 200 μg/ml, or about 20 μg/ml to about 100 μg/ml drug, about 20 μg/ml, about 40 μg/ml, about 60 μg/ml, about 80 μg/ml, about 100 μg/ml, about 120 μg/ml, about 140 μg/ml, about 160 μg/ml, about 180 μg/ml, or about 200 μg/ml.

When administering a modulator to a host which has already been administered the ligand, the ratio of modulator to ligand can be adjusted based on the desired level of inhibition of the ligand. The modulator dose can be calculated based on correlation with the dose of ligand. In one embodiment, the weight-to-weight dose ratio of modulator to ligand is 1:1. In other embodiments, the ratio of modulator to ligand is greater than 1:1 such as 2:1 or about 2:1, 3:1 or about 3:1, 4:1 or about 4:1, 5:1 or about 5:1, 6:1 or about 6:1, 7:1 or about 7:1, 8:1 or about 8:1, 9:1 or about 9:1, 10:1 or about 10:1 or more. In other embodiments, the weight-to-weight dose ratio of modulator to ligand is less than about 1:1 such as 0.9:1 or about 0, 9:1, 0.8:1 or about 0.8:1, 0.7:1 or about 0.7:1, 0.6:1 or about 0.6:1, 0.5:1 or about 0.5:1, 0.45:1 or about 0.45:1, 0.4:1 or about 0, 4:1, 0.35:1 or about 0.35:1, 0.3:1 or about 0.3:1, 0.25:1 or about 0, 25:1, 0.2:1 or about 0.2:1, 0.15:1 or about 0.15:1, 0.1:1 or about 0.1:1 or less than 0.1:1 such as about 0.005:1 or less. In some embodiments, the weight-to-weight dose ratio is between 0.5:1 and 0.1:1, or between 0.5:1 and 0.2:1, or between 0.5:1 and 0.3:1. In other embodiments, the weight-to-weight dose ratio is between 1:1 and 5:1, or between 1:1 and 110:1, or between 1:1 and 20:1.

The ligands can be administered intravenously in a single daily dose, an every other day dose, or the total daily dosage can be administered in several divided doses. Ligand and/or modulator administration may be provide once per day (q.d.), twice per day (b.i.d.), three times per day (t.i.d.) or more often as needed. Thereafter, the modulator is provided by any suitable means to alter the effect of the nucleic acid ligand by administration of the modulator, Nucleic acid ligands can be administered subcutaneously twice weekly, weekly, every two weeks or monthly. In some embodiments, the ligand or modulators are administered less often than once per day. For example, ligand administration may be carried out every other day, every three days, every four days, weekly, or monthly.

In one embodiment, co-administration or sequential administration of other agents can be desirable. For combination treatment with more than one active agent, where the active agents are in separate dosage formulations, the active agents can be administered concurrently, or they each can be administered at separately staggered times.

The compositions are administered in a manner compatible with the dosage formulation, and in a therapeutically effective amount. The quantity to be administered depends on the host to be treated, capacity of the hoses system to utilize the active ingredient, and degree of therapeutic effect desired. Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are provided.

In certain embodiments, the compositions of this disclosure may further comprise another therapeutic agent. When a second agent is used, the second agent may be administered either as a separate dosage form or as part of a single dosage form with the compounds or compositions. While one or more of the inventive compounds can be used in an application of monotherapy to treat a disorder, disease or symptom, they also may be used in combination therapy, in which the use of an inventive compound or composition (therapeutic agent) is combined with the use of one or more other therapeutic agents for treating the same and/or other types of disorders, symptoms and diseases. Combination therapy includes administration of the two or more therapeutic agents concurrently or sequentially. The agents may be administered in any order. Alternatively, the multiple therapeutic agents can be combined into a single composition that can be administered to the patient.

### Methods for Characterizing CLEC-2 Ligands and Modulators of CLEC-2 Ligands

In one aspect, the present disclosure provides methods for characterizing CLEC-2 gands which may function as activators or inhibitors of CLEC-2. Such methods assess CLEC-2 activity in terms of CLEC-2-mediated platelet aggregation. In one embodiment, the assays are done in vitro or ex vivo using whole blood flow-based platelet adhesion assays.

Various studies suggest that while CLEC-2 may not be required for the initial platelet adherence to collagen-coated surfaces, CLEC-2 may be required for stabilization of the platelet aggregates and thrombus growth. The role of CLEC-2 in thrombosis has been studied, for example, using CLEC-2 chimeras in mice. These studies suggest that CLEC-2 is involved in thrombus stabilization both in vitro and in vivo (Suzuki-Inoue, K. et al., 2010, J. Biol. Chem. 285, 24494-24507). The role of the CLEC-2 receptor in platelet aggregate stabilization in vitro was also recently demonstrated using a whole blood perfusion assay, where CLEC-2 depleted platelets were able to adhere normally to a collagen coated surface, but subsequent stable platelet aggregate formation was strongly impaired under intermediate to high shear rates (May, F. et. al., 2009 Blood, 114:3464-3472). Through such studies, it has been hypothesized that CLEC-2 may contribute to thrombus growth by ‘taking over’ the function of GPVI in platelets recruited to a growing thrombus that will not get contact with collagen (Neswandt, B. et. al., 2011, J. Throm. Haemost, suppl 1:92-104). In other words, while adherence of platelets to a collagen-coated surface and initial thrombus formation depends on GPVI, further aggregation of platelets, i.e., to form a thrombus, requires CLEC-2.

Adhesion of platelets under flow conditions on the collagen coated surfaces have been used routinely for studying in vitro and in vivo thrombus growth and stability. These flow experiments are done, for example, using an instrument such as BioFlu™ (Fluxion Biosciences, South San Francisco, Calif.), which provides the ability to emulate physiological shear flow in an in vitro model, white also offering a high throughput well plate format.

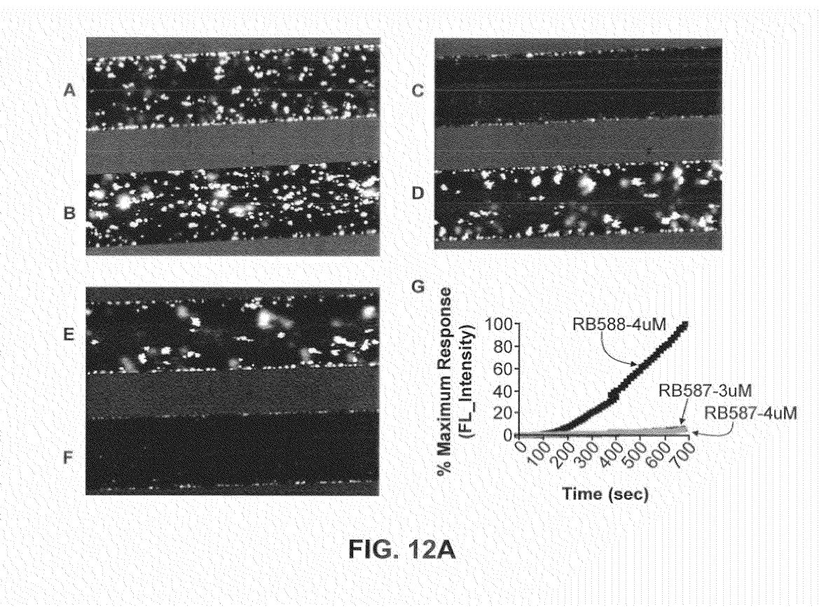

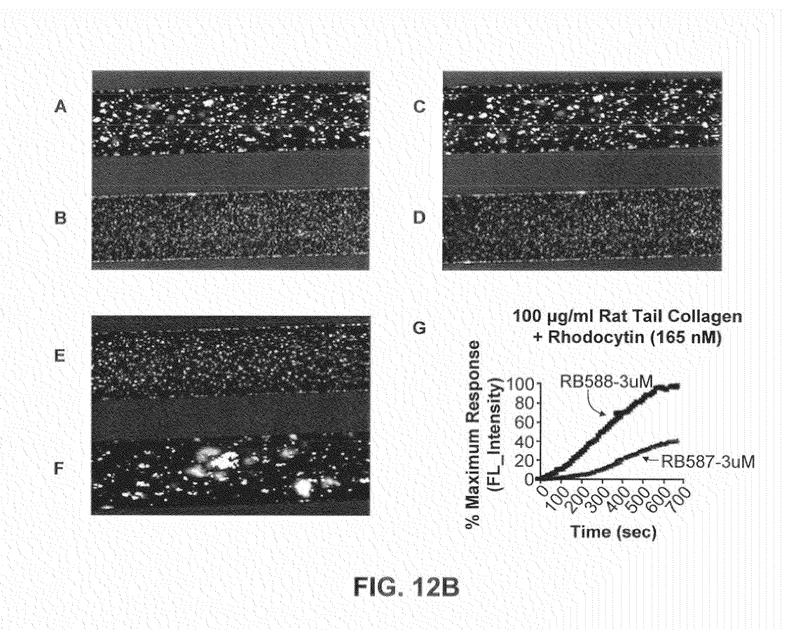

It was presently discovered that in vitro flow-based platelet adhesion assays using whole blood could be used to identify activators or inhibitors of CLEC-2-mediated platelet aggregate formation. In one embodiment, the surface which is subjected to shear flow of whole blood is coated with rhodocytin, which is known to effectively stimulate CLEC-2 dependent platelet aggregate formation. As detailed in Example 10 (FIG. 12A), use of a rhodocytin coated surface supports the accumulation of activated platelets in thrombi under flow conditions in whole blood. The assay method is then performed in the presence of putative inhibitors or activators of CLEC-2 dependent platelet aggregate formation. For example, the whole blood sample is incubated with a compound known to bind CLEC-2 (e.g., any one of the CLEC-2 ligands described herein). A comparison can then be made of the platelet aggregate formation in the presence and in the absence of the CLEC-2 ligand. An inhibitor of CLEC-2 dependent platelet aggregate formation is a CLEC-2 ligand which reduces the level of platelet aggregate formation by at least 50%, 75%, 80%, 85%, 90%, or 95% as compared to the level of platelet aggregate formation in the absence of the CLEC-2 ligand.

This assay may also be used to identify modulators of the CLEC-2 ligand. Again, as described in Example 10, the whole blood sample may be incubated in the presence of a CLEC-2 ligand in the further presence or absence of a modulator of the CLEC-2 ligand. A compound which is a functional modulator of the CLEC-2 ligand will reverse the activity of the CLEC-2 ligand. For example, if the CLEC-2 ligand has reduced the level of platelet aggregate formation, addition of the functional modulator of the CLEC-2 will result in an increase in platelet aggregate formation relative to the level seen in the presence of the CLEC-2 ligand only.

In a second protocol useful for characterizing the function of a CLEC-2 ligand, an in vitro flow-based platelet adhesion assays using whole blood is performed wherein the whole blood is exposed to a surface coated with soluble collagen type I (collagen I). For example, Example 10 (FIG. 12B), shows assays performed using the BIOFLUX, in which wells were coated with soluble rat-tail collagen I. In this protocol, the whole blood sample is incubated with rhodocytin prior to flow past the soluble collagen I coated surface. The presence of rhodocytin in the sample resulted in the formation of larger platelet aggregates on the soluble collagen I coated surface compared to platelet aggregates formed upon flow of blood lacking rhodocytin. To test the ability of a CLEC-2 ligand to activate or inhibit CLEC-2 mediated platelet aggregate formation, the whole blood sample is incubated with a CLEC-2 ligand prior to the blood flow step. Incubation with the CLEC-2 ligand can be done prior to or after incubation with the rhodocytin. Alternatively, the CLEC-2 ligand and rhodocytin are added simultaneously to the blood sample. As above, a comparison can then be made of the platelet aggregate formation in the presence and in the absence of the CLEC-2 ligand. An inhibitor of CLEC-2 dependent platelet aggregate formation is a CLEC-2 ligand which reduces the level of platelet aggregate formation by at least 50%, 75%, 80%, 85%, 90%, or 95% as compared to the level of platelet aggregate formation in the absence of the CLEC-2 ligand. Further, a modulator of the CLEC-2 ligand can be characterized for its ability to reverse the effects of the CLEC-2 ligand on CLEC-2-mediated platelet aggregate formation, as described above. A compound which is a functional modulator of the CLEC-2 ligand will reverse the activity of the CLEC-2 ligand. For example, if the CLEC-2 ligand has reduced the level of platelet aggregation, addition of die functional modulator of the CLEC-2 ligand will result in an increase in platelet aggregate formation relative to the level seen in the presence of the CLEC-2 ligand only.

It is understood that these assay methods for characterizing functional activity of CLEC-2 ligands and modulators of CLEC-2 ligands, are not limited to use with whole blood samples. The blood sample may, in certain embodiments, include one or more natural blood components. The blood sample may, in certain embodiments, include one or more artificial blood components. Alternatively, the blood sample as used in the flow assay is a sample which comprises whole blood or platelet-rich plasma or washed platelets.

The functional activity of CLEC-2 ligands and modulators could be tested using various cell lines, for example, endothelial cells, dendritic cells, leukocytes, monocytes, neutrophils, macrophases, lymphoid cells, immune cells i.e., Natural Killer T cells, B cells. Various functional assays including platelet-platelet interactions, platelet-monocyte interactions, platelet leukocyte interactions, calcium signaling, cell adhesion assays, secretion assays, LPS induced inflammation assays, cell migration assays, Syk and Srk phosphorylation assays, ELISA assays for ligand binding, flow cytometry based receptor occupancy assays, or diagnostic markers assays. The reagents and synthetic equivalents of CLEC-2 receptor modulator that could be tested with the aptamer ligands are cross-linked antibodies, homophilic interaction studies of CLEC-2 receptors, small molecule inhibitors and activators, peptides, proteins, monoclonal antibodies, lipoproteins, lipidated peptides etc.

The following examples are provided to illustrate certain aspects of the present disclosure. These examples are in no way to be considered to limit the scope of the disclosure.

### EXAMPLES

### Example 1

### Identification of Nucleic Acid Ligands to CLEC-2

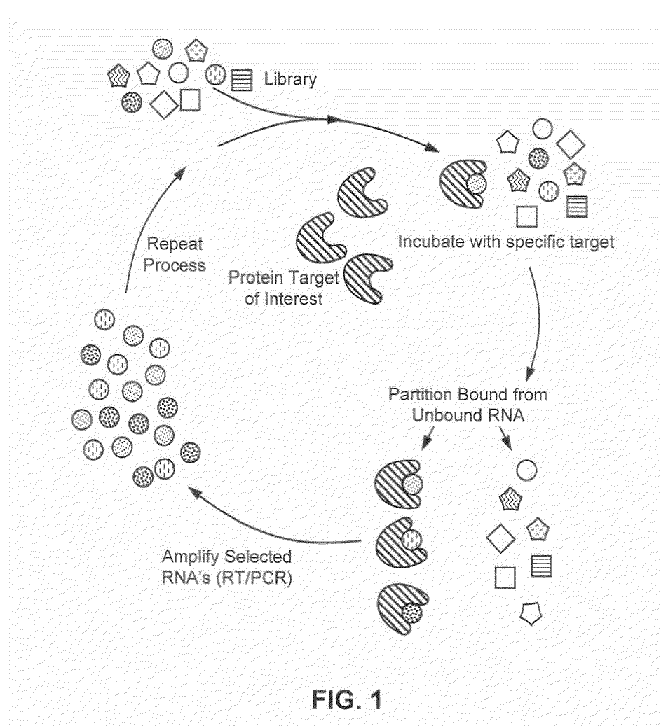

The SELEX method was used to obtain ligands which bind CLEC-2 as described and illustrated in FIG. 1.

Candidate DNA libraries were generated by heat annealing and snap-cooling 1 nmole of template DNA oligo and 1.5 nmoles of 5′ DNA primer ago. The sequences of the Sel2 DNA template oligo for designing the candidate mixture are: 5′-TCTCGGATCC TCAGCGAGTC GTCTG(N40)CCGCA TCGTCCTCCC TA-3′ (SEQ ID NO:4) (N40 represents 40 contiguous nucleotides synthesized with equimolar quantities of A, T, G and C), the 5′ primer oligo and 3′ primer oligo are, respectively, 5′-GGGGGAATTC TAATACGACT CACTATAGGG AGGACGATGC GG-3′ (T7 promoter sequence is in bold), and 5′-TCTCGGATCC TCAGCGAGTC GTCTG-3′. The reactions were filled in with Exo-Klenow, stopped by addition of EDTA to a final concentration of 2 mM, and extracted with PCI [phenol:chloroform:isoamyl alcohol (25:24:1)] and then chloroform:isoamyl alcohol (24:1). The extract was desalted, concentrated, and unincorporated nucleotides removed with an Amicon 10 spin column. The DNA templates were utilized in a transcription reaction to generate a 2′-fluoropyrimidine starting library. In vitro transcription conditions were 40 mM Tris-HCl pH 8.0, 4% PEG-800, 12 mM MgCl₂, 1 mM spermidine, 0.002% Triton, 5 mM DTT, 1 mM rGTP, 1 mM rATP, 3 mM 2′F-CTP, 3 mM 2′F-UTP, 8 μg/mL inorganic pyrophosphatase, 0.5 μM DNA library, and Y639F mutant T7 polymerase. Transcriptions were incubated overnight at 37° C., DNase treated, chloroform:isoamyl alcohol (24:1) extracted twice, concentrated with an Amicon 10 spin column, and gel purified on a 12% denaturing PAGE gel. RNA was eluted out of the gel, and buffer exchanged and concentrated with TE (10 mM Iris pH 7.5, 0.1 mM EDTA) washes in an Amicon 10 spin column.

The CLEC-2 selection started with a complex library of ˜10¹⁴ different 2′-fluoropyrimidine RNA sequences. The complex RNA pool was precleared against a biotin-PEG6-His6 peptide, immobilized on magnetic streptavidin beads. The precleared RNA was bound to the purified recombinant N-term His10 tagged CLEC-2 protein (SEQ ID NO:3). Purified histidine-tagged CLEC-2 protein was obtained from R&D Systems (Minneapolis, Minn.), Catalog No. 1718-CL-050, and encompassed residues Gln58-Pro229.

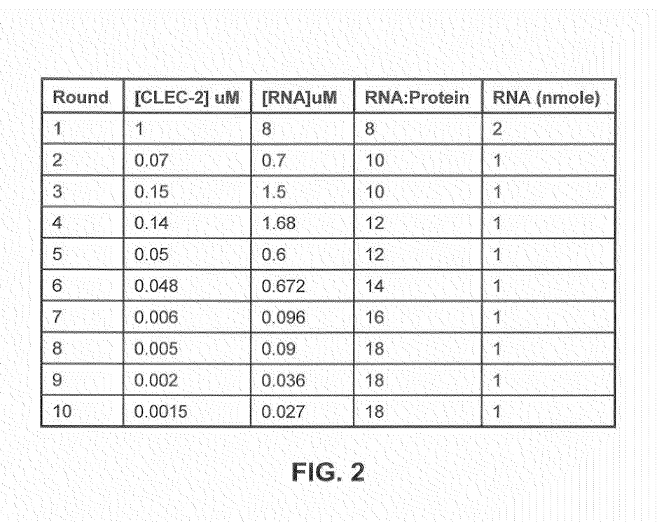

CLEC-2 ligand selection was performed in binding buffer “F.” Binding buffer F consists of 20 mM HEPES pH 7.4, 150 mM NaCl, 2 mM CaCl₂, and 0.01% BSA. Starting in Round 3, 0.0024% yeast tRNA was included in the round binding reactions. Protein-RNA complexes were partitioned over a 25 mm nitrocellulose disc with washing. The bound RNA was extracted off the nitrocellulose disc with incubation in PCI (25:24:1), Water was added and the aqueous phase extracted, followed by a chloroform:isoamyl alcohol (24:1) extraction. The resultant bound RNA was ethanol precipitated. One quarter of the precipitated RNA was heat annealed to the 3′ primer and reverse transcribed using AMY RT. The entire RT reaction was used in a PCR with 5 and 3′ primers and standard PCR conditions to generate the DNA template for the next round of RNA generation. Specific conditions for each round of selection are shown in FIG. 2.

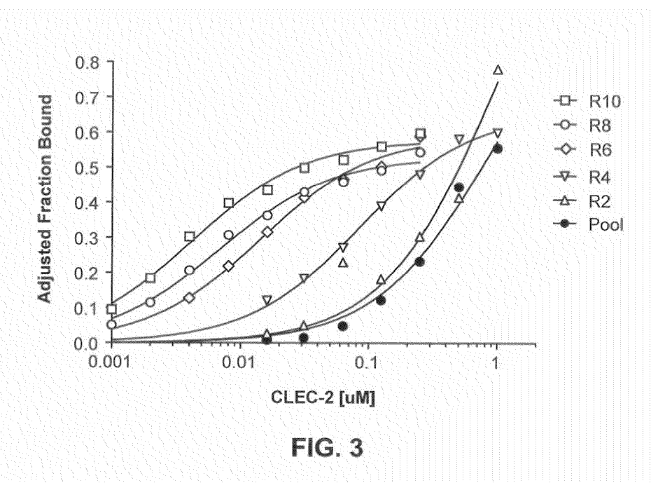

Enrichment of the ligand libraries for CLEC-2 was monitored in direct binding studies utilizing radiolabeled ligand RNA pools from respective rounds of SELEX and soluble CLEC-2, Binding studies were performed with trace P³² end-labeled RNA added to serial dilutions of CLEC-2 in binding buffer F. To prepare radiolabeled RNAs for binding studies, one hundred picomoles of RNA was dephosphorylated with Bacterial Alkaline Phosphatase at 50° C. for 1 hour. The reaction was phenol:chloroform:isoamyl alcohol (25:24:1) extracted, chloroform: isoamyl alcohol (24:1) extracted, and ethanol precipitated. Three pmoles of dephosphorylated RNA was end labeled with T4 Polynucleotide Kinase with supplied buffer, and 20 μCi. of γ-P³²-ATP and subsequently cleaned with a Biorad MicroBio Spin P-30 spin column. End-labeled RNA was diluted to a final concentration of 2000 cpm/μL and heat denatured at 65° C. for 5 minutes. RNA and CLEC-2 dilutions were equilibrated at 37° C. prior to use. RNA (5 μL) was added to varying concentrations of CLEC-2 (15 μL) at 37° C. and incubated together for 5 to 15 min. The complexed RNA/CLEC-2 protein mixture was then loaded over a Protran BA85 nitrocellulose membrane, overlayed on a Genescreen Plus Nylon membrane, in a 96 well vacuum manifold system with washing. The membranes were exposed to a phosphorimager screen, scanned, and quantitated with a Molecular Dynamics Storm 840 Phosphorimager. The fraction bound was calculated by dividing the counts on the nitrocellulose by the total counts and adjusting for the background. The enrichment of binding of the CLEC-2 selection is shown in FIG. 3.

### Example 2

### Sequencing and Identification of CLEC-2 Nucleic Acid Ligands

The final PCR products representing anti-CLEC-2 enriched ligand libraries from Round 10 of the SELEX experiments described in Example 1 were digested with EcoR1 and BamH1, cleaned using a purification kit, and directionally cloned into linearized pUC19 vector. Bacterial colonies were streaked for single clones and 5 mL overnight cultures were inoculated from single colonies. Plasmid DNA was prepared from single colonies using Invitrogen Pure link Quick Plasmid Miniprep kits and sequenced utilizing a vector primer. A total of 42 clones were sequenced, with 20 representing unique sequences. Each of these unique sequences was evaluated with respect to CLEC-2 affinity (according the methods in Example 1) and ability to rhodocytin-induced CLEC-2-dependent platelet aggregation (according the methods in Example 8). After analysis of the resultant data for the 20 clones, the ligand S2-20 was identified as a high affinity inhibitor of CLEC-2-dependent aggregation, and was characterized more fully as detailed below.

The sequences of the DNA random region, the corresponding RNA random region, the length DNA, the full length RNA, and the full length RNA with modifications for aptamer S2-20 are shown in Table 1. Full-length refers to sequences resulting from the SELEX process, comprising sequences derived from both the random portion of the ligand libraries used in the SELEX process as well as sequences from the fixed sequence portions flanking the random region.

TABLE 1
Sequences for CLEC-2 Ligand S2-20

| | | SEQ |
|---|---|---|
| | | ID |
| Clone Name | Sequence | NO: |
| S2-20 Random | ACTGGGCTCATATTCCCGCGCATCTTACTACCTCGGTTAT | 5 |
| Region DNA | | |
| Sequence | | |
| S2-20 Random | ACUGGGCUCAUAUUCCCGCGCAUCUUACUACCUCGGUUAU | 6 |
| Region RNA | | |
| Sequence | | |
| S2-20 Full | GGGAGGACGATGCGGACTGGGCTCATATTCCCGCGCATCTTACTACCTCGGTTAT | 7 |
| Length DNA | CAGACGACTCGCTGAGGATCCGAGA | |
| Sequence | | |
| S2-20 Full | GGGAGGACGAUGCGGACUGGGCUCAUAUUCCCGCGCAUCUUACUACCUCGGUUAU | 8 |
| Length RNA | CAGACGACUCGCUGAGGAUCCGAGA | |
| Sequence | | |
| S2-20 Full | rGrGrGrArGrGrAfCrGrAfUrGfCrGrGrAfCfUrGrGrGfCfUfCrAfUrAf | 9 |
| Length RNA | UfUfCfCfCrGfCrGfCrAfUfCfUfUrAfCfUrAfCfCfUfCrGrGfUfUrAfU | |
| Sequence with | fCrArGrAfCrGrAfCfUfCrGfCfUrGrArGrGrAfUfCfCrGrArGrA | |
| modifications | | |

SEQ ID NOs refer to the unmodified versions of the ligands described in the column titled, “Modified Sequence”; Sequences are listed in a 5′-3′ direction
rG = 2′Ribo G; rA = 2′Ribo A; fC = 2′-Fluoro C; fU = 2′-Fluoro U

The affinity of the anti-CLEC-2 ligand for CLEC-2 was determined by direct binding studies using radiolabeled trace ligand RNA and soluble CLEC-2 as described above in Example 1. The affinity of the anti-CLEC-2 ligand, S2-20, for CLEC-2 was high, with a Kd of ˜1.4 nM.

### Example 3

### Truncation Probing of Anti-CLEC-2 Ligand Sequence

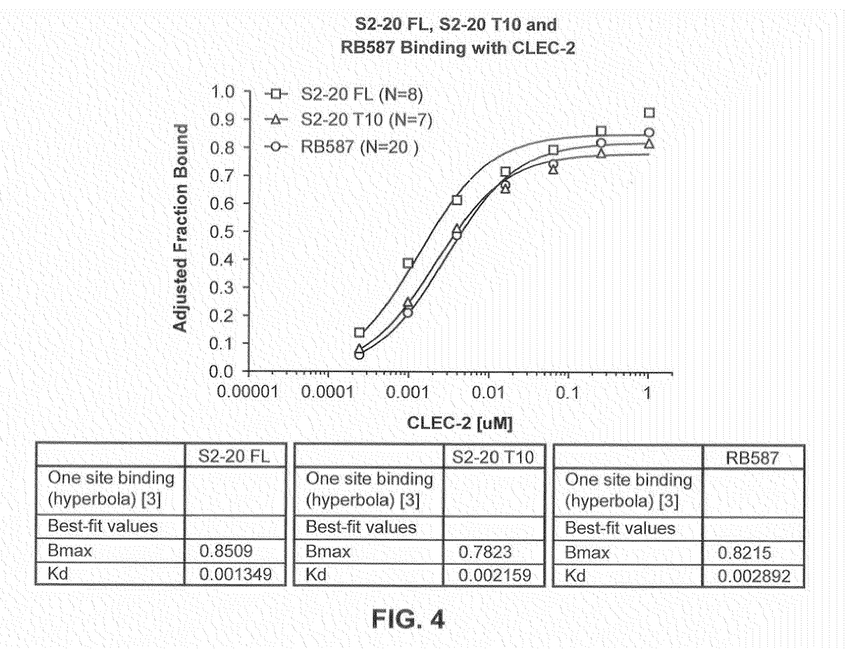

Screening of S2-20 for potential secondary structure was conducted utilizing the mfold server (mfold.bioinfo.rpi.edu). A description of these methods is found on the server site as well as in M. Zuker (2003) “Mfold web server for nucleic acid folding and hybridization prediction.” Nucleic Acids Res. 31 (13), 3406-15 and D. H. Mathews, et al. (1999) “Expanded Sequence Dependence of Thermodynamic Parameters Improves Prediction of RNA Secondary Structure” J. Mol. Biol. 288, 911-940. A series of truncated compounds for the anti-CLEC-2 ligand S2-20 were generated (Table 2), and their affinity for CLEC-2 determined (Table 2). With the exception of RB 587, all truncates were transcribed in vitro from DNA templates. RB587 was synthesized on a Mermade 12 DNA/RNA Synthesizer with idT preloaded CPG. Binding data for the S2-20 full-length aptamer, the S2-20 T10 truncate, and RB587 are shown in FIG. 4.

TABLE 2
S2-20 Truncations

| | | | SEQ |
|---|---|---|---|
| Clone | | Kd | ID |
| Name | RNA Sequence | (nM) | NO: |
| S2-20 | rGrGrGrArGrGrAfCrGrAfUrGfCrGrGrAfCfUrGrGrGfCfUfCrAfUrAfUfUfC | 1.4 | 10 |
| | fCfCrGfCrGfCrAfUfCfUfUrAfCfUrAfCfCfUfCrGrGfUfUrAfUfCrArGrAfC | | |
| | rGrAfCfUfCrGfCfUrGrArGrGrAfUfCfCrGrArGrA | | |
| S2-20 T1 | rGrGrGrArGrGrAfCrGrAfUrGfCrGrGrAfCfUrGrGrGfCfUfCrAfUrAfUfUfC | 4.6 | 11 |
| | fCfCrGfCrGfCrAfUfCfUfUrAfCfUrAfCfCfUfCrGrGfUfUrAfU | | |
| S2-20 T2 | rGrGrGrArGrGrAfCrGrAfUrGfCrGrGrAfCfUrGrGrGfCfUfCrAfUrAfUfUfC | 2.8 | 12 |
| | fCfCrGfCrGfCrAfUfCfUfUrAfCfUrAfCfCfUfC | | |
| S2-20 T3 | rGrGrGrArGrGrAfCrGrAfUrGfCrGrGrAfCfUrGrGrGfCfUfCrAfUrAfUfUfC | 2.2 | 13 |
| | fCfCrGfCrGfCrAfUfCfUfUrAfCfUrA | | |
| S2-20 T4 | rGrGrGrArGrGrAfCrGrAfUrGfCrGrGrAfCfUrGrGrGfCfUfCrAfUrAfUfUfC | 1.4 | 14 |
| | fCfCrGfCrGfCrAfUfC | | |
| S2-20 T5 | rGrGrGrArGrGrAfCrGrAfUrGfCrGrGrAfCfUrGrGrGfCfUfCrAfUrAfUfUfC | dead | 15 |
| | fCfCrG | | |
| S2-20 T6 | rGrGrGrA--------rGrAfUrGfCrGrGrAfCfUrGrGrGfCfUfCrAfUrAfUfUfC | 1 | 16 |
| | fCfCrGfCrGfCrAfUfC | | |
| S2-20 T6 | rGrGrGrA--------rGrAfUrGfCrGrGrAfCfUrGrGrGfCfUfCrAtUrAfUfUfC | 3.9 | 17 |
| ext8bp | fCfCrGfCrGfCrAfUfCfUfC | | |
| S2-20 T6 | rGrGrGrA--------rGrAfUrGfCrGrGrAfCfUrGrGrGfCfUfCrAfUrAfUfUfC | 4.6 | 18 |
| ext10bp | fCfCrGfCrGfCrAfUfCfUfCfCfC | | |
| S2-20 T7 | rGrGrGrA--------rGrA--rGfCrGrGrAfCfUrGrGrGfCfUfCrAfUrAfUfUfC | 3 | 19 |
| | fCfCrGfCrGfC--fUfC | | |
| S2-20 T8 | rGrGrGrA--------rGrAfU--fCrGrGrAfCfUrGrGrGfCfUfCrAfUrAfUfUfC | 1.6 | 20 |
| | fCfCrGfCrG--rAfUfC | | |
| S2-20 T9 | rGrGrGrA--------rGrAfUrG--rGrGrAfCfUrGrGrGfCfUfCrAfUrAfUfUfC | 18.7 | 21 |
| | fCfCrGfC--fCrAfUfC | | |
| S2-20 | rGrGrGrA------------fUrGfCrGrGrAfCfUrGrGrGfCfUfCrAfUrAfUfUfC | 2.2 | 22 |
| T10 | fCfCrGfCrGfCrAfUfC | | |
| S2-20 | rGrGrGrA------------fUrGfCrGrGrAfCfCrGrGrGfCfUfCrAfUrAfUfUfC | 66 | 23 |
| T11 | fCfCrGfCrGfCrAfUfC | | |
| RB 587 | ----rGrA------------fUrGfCrGrGrAfCfUrGrGrGfCfUfCrAfUrAfUfUfC | 2.9 | 24 |
| | fCfCrGfCrGfCrAfUfCidT | | |

SEQ ID NOs refer to the unmodified versions of the ligands described in the column titled, “Modified Sequence”; Sequences are listed in a 5′-3′ direction
rG = 2′Ribo G; rA = 2′Ribo A; fC = 2′-Fluoro C; fU = 2′-Fluoro U

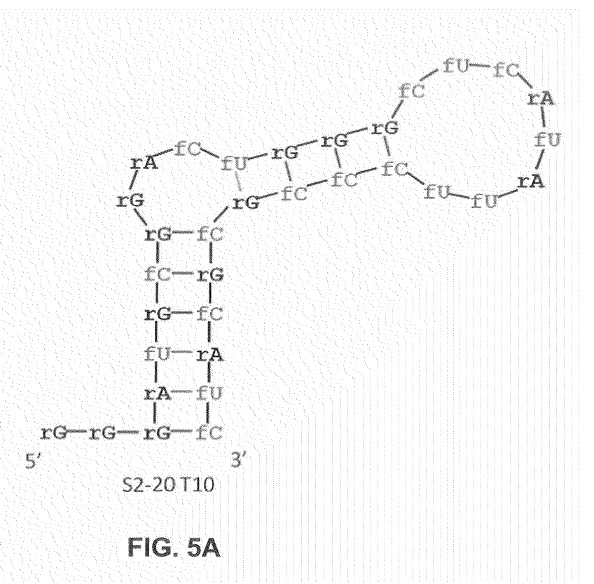

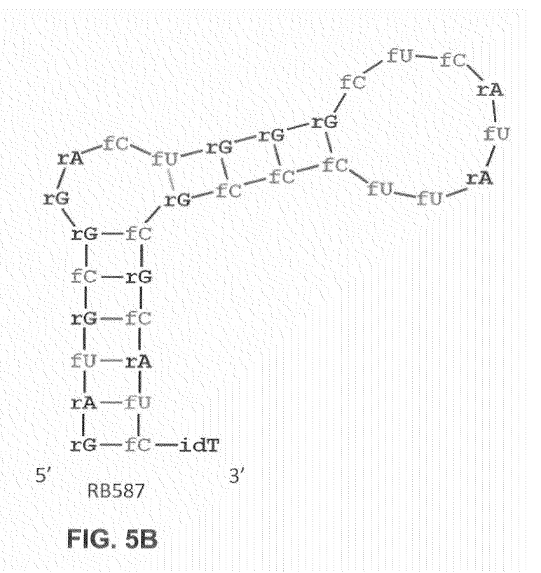

The secondary structure of truncates S2-20 T10 and RB587 are presented in FIG. 5. The predicted boundaries of the 5′ and 3′ ends are sufficient to form Stem 1 and functional anti-CLEC-2 ligands. If the 3′ portion of stem 1 is removed, as in S2-20 T5, binding to CLEC-2 is abolished. The binding data for S2-20 T7 and S2-20 T8 indicates that stem 1 can be 5 base pairs in length and still retain comparable binding affinity. Lengthening of stem 1, from 6 base pairs to 8-10 base pairs, as demonstrated with S2-20 T6 ext8 and S2-20 T6 ext10, also bound CLEC-2 with high affinity. Thus, stem 1 can be 5-10 base pairs in length with comparable binding affinities.

Stem 2 is a four base pair stem with a wobble base pair at the bottom being preferred. S2-20 T11 has a single substitution changing the bottom base pair of stem 2 from a U-G to a C-G. This substitution is not well tolerated as demonstrated by the significant (approximately 30 fold) increase in Kd.

### Example 4

### Mutational Probing of Anti-CLEC-2 Ligand Sequence

A series of mutant compounds for S2-20 truncates containing nucleotide mutations within loop regions were generated and their binding affinity for CLEC-2 was determined (Table 3). With the exception of RB588, all mutant truncates were transcribed in vitro from DNA templates. RB588 was synthesized on a Mermade 12 DNA/RNA Synthesizer with idT preloaded CPG.

TABLE 3
S2-20 Mutations

| | | | SEQ |
|---|---|---|---|
| Clone | | Kd | ID |
| Name | RNA Sequence | (nM) | NO: |
| S2-20 | rGrGrGrA---- | >60 | 25 |
| Mut1 | rGrAfUrGfCrGfCfUfCfUrGrGrGfCfUfCrAfUrAfUfUfCfCfCrGfCrGfCrAfUfC | | |
| S2-20 | rGrGrGrA---- | >60 | 26 |
| Mut2 | rGrAfUrGfCrGrGrAfCfUrGrGrGrGrAfCrAfUrAfUfUfCfCfCrGfCrGfCrAfUfC | | |
| S2-20 | rGrGrGrA---- | >60 | 27 |
| Mut3 | rGrAfUrGfCrGrGrAfCfUrGrGrGfCfUrGfUfUrAfUfUfCfCfCrGfCrGfCrAfUfC | | |
| S2-20 | rGrGrGrA---- | >60 | 28 |
| Mut4 | rGrAfUrGfCrGrGrAfCfUrGrGrGfCfUfCrArAfUfUfUfCfCfCrGfCrGfCrAfUfC | | |
| S2-20 | rGrGrGrA---- | >60 | 29 |
| Mut5 | rGrAfUrGfCrGrGrAfCfUrGrGrGfCfUfCrAfUrArArAfCfCfCrGfCrGfCrAfUfC | | |
| S2-20 | rGrGrGrA---- | >60 | 30 |
| Mut6 | rGrAfUrGfCrGrGrArGfUrGrGrGfCfUfCrAfUrAfUfUfCfCfCrGfCrGfCrAfUfC | | |
| S2-20 | rGrGrGrA---- | >60 | 31 |
| Mut7 | rGrAfUrGfCrGfCrAfCfUrGrGrGfCfUfCrAfUrAfUfUfCfCfCrGfCrGfCrAfUfC | | |
| RB 588 | ------------ | >60 | 32 |
| | rGrAfUrGfCrGfCrAfCfUrGrGrGfCfUfCrAfUrAfUfUfCfCfCrGfCrGfCrAfU | | |
| | fCidT | | |
| S2-20 | rGrGrGrA---- | 3.5 | 33 |
| Mut8 | rGrAfUrGtCrGrGfUfCfUrGrGrGfCfUfCrAfUrAfUfUfCfCfCrGfCrGfCrAfUfC | | |
| S2-20 | rGrGrGrA------ | >60 | 34 |
| T10 | fUrGfCrGrGrAfCfUrGrGrGrGfUfCrAfUrAfUfUfCfCfCrGfCrGfCrAfUfC | | |
| Mut9 | | | |
| S2-20 | rGrGrGrA------ | >60 | 35 |
| T10 | fUrGfCrGrGrAfCfUrGrGrGfCrAfCrAfUrAfUfUfCfCfCrGfCrGfCrAfUfC | | |
| Mut10 | | | |
| S2-20 | rGrGrGrA------ | >60 | 36 |
| T10 | fUrGfCrGrGrAfCfUrGrGrGfCfUrGrAfUrAfUfUfCfCfCrGfCrGfCrAfUfC | | |
| Mut11 | | | |
| S2-20 | rGrGrGrA------ | 2.4 | 37 |
| T10 | fUrGfCrGrGrAfCfUrGrGrGfCfUfCfUfUrAfUfUfCfCfCrGfCrGfCrAfUfC | | |
| Mut12 | | | |
| S2-20 | rGrGrGrA------ | 2.4 | 38 |
| T10 | fUrGfCrGrGrAfCfUrGrGrGfCfUfCrArArAfUfUfCfCfCrGfCrGfCrAfUfC | | |
| Mut13 | | | |
| S2-20 | rGrGrGrA------ | >60 | 39 |
| T10 | fUrGfCrGrGrAfCfUrGrGrGfCfUfCrAfUfUfUfUfCfCfCrGfCrGfCrAfUfC | | |
| Mut14 | | | |
| S2-20 | rGrGrGrA------ | 30 | 40 |
| T10 | fUrGfCrGrGrAfCfUrGrGrGfCfUfCrAfUrArAfUfCfCfCrGfCrGfCrAfUfC | | |
| Mut15 | | | |
| S2-20 | rGrGrGrA------ | >60 | 41 |
| T10 | fUrGfCrGrGrAfCfUrGrGrGfCfUfCrAfUrAfUrAfCfCfCrGfCrGfCrAfUfC | | |
| Mut16 | | | |
| S2-20 | rGrGrGrA------ | 8.8 | 42 |
| T10 | fUrGfCrGrGrAfCfUrGrGrGfUfUfCrAfUrAfUfUfCfCfCrGfCrGfCrAfUfC | | |
| Mut17 | | | |
| S2-20 | rGrGrGrA------ | 51.7 | 43 |
| T10 | fUrGfCrGrGrAfCfUrGrGrGfCfCfCrAfUrAfUfUfCfCfCrGfCrGfCrAfUfC | | |
| Mut18 | | | |
| S2-20 | rGrGrGrA------ | 5.7 | 44 |
| T10 | fUrGrCrGrGrAfCfUrGrGrGfCfUfUrAfUrAfUfUfCfCfCrGfCrGfCrAfUfC | | |
| Mut19 | | | |
| S2-20 | rGrGrGrA------ | 1.2 | 45 |
| T10 | fUrGfCrGrGrAfCfUrGrGrGfCfUfCrGfUrAfUfUfCfCfCrGfCrGfCrAfUfC | | |
| Mut20 | | | |
| S2-20 | rGrGrGrA------ | 1.7 | 46 |
| T10 | fUrGfCrGrGrAfCfUrGrGrGfCfUfCrAfCrAfUfUfCfCfCrGfCrGfCrAfUfC | | |
| Mut21 | | | |
| S2-20 | rGrGrGrA------ | 5.6 | 47 |
| T10 | fUrGfCrGrGrAfCfUrGrGrGfCfUfCrAfUrGfUfUfCfCfCrGfCrGfCrAfUfC | | |
| Mut22 | | | |
| S2-20 | rGrGrGrA------ | 6.7 | 48 |
| T10 | fUrGfCrGrGrAfCfUrGrGrGfCfUfCrAfUrAfCfUfCfCfCrGfCrGfCrAfUfC | | |
| Mut23 | | | |
| S2-20 | rGrGrGrA------ | >60 | 49 |
| T10 | fUrGfCrGrGrAfCfUrGrGrGfCfUfCrAfUrAfUfCfCfCfCrGfCrGfCrAfUfC | | |
| Mut24 | | | |
| S2-20 | rGrGrGrA------ | >60 | 50 |
| T10 | fUrGfCrGrArAfCfUrGrGrGfCfUfCrAfUrAfUfUfCfCfCrGfCrGfCrAfUfC | | |
| Mut25 | | | |
| S2-20 | rGrGrGrA------ | >60 | 51 |
| T10 | fUrGfCrGrGrAfUfUrGrGrGfCfUfCrAfUrAfUfUfCfCfCrGfCrGfCrAfUfC | | |
| Mut26 | | | |
| S2-20 | rGrGrGrA------ | >60 | 52 |
| T10 | fUrGfCrGrGrAfCfUrGrGrGfCfUfCrAfUfCfUfUfCfCfCrGfCrGfCrAfUfC | | |
| Mut27 | | | |
| S2-20 | rGrGrGrA------fUrGfCrGrGrAfCfUrGrGrGfCfUfC- | >60 | 53 |
| T10 | fUrAfUfUfCfCfCrGfCrGfCrAfUfC | | |
| Mut28 | | | |
| S2-20 | rGrGrGrA------fUrGfCrGrGrAfCfUrGrGrGfCfUfCrAfU- | >60 | 54 |
| T10 | fUfUfCfCfCrGfCrGfCrAfUfC | | |
| Mut29 | | | |
| S2-20 | rGrGrGrArGrGrAfCrGrAfUrGfCrGrGfUfCfUrGrGrGfCfUfCrAfUrAfUfUfCfC | 6.8 | 55 |
| T4 | fCrGfCrGfCrAfUfC | | |
| Mut30 | | | |
| S2-20 | rGrGrGrArGrGrAfCrGrAfUrGfCrGrGrGfCfUrGrGrGfCfUfCrAfUrAfUfUfCfC | 4.3 | 56 |
| T4 | fCrGfCrGfCrAfUfC | | |
| Mut31 | | | |
| S2-20 | rGrGrGrArGrGrAfCrGrAfUrGfCrGrGrAfCfUrGrGrGfCfUfCrArGrAfUfUfCfC | 12.4 | 57 |
| T4 | fCrGfCrGfCrAfUfC | | |
| Mut32 | | | |
| S2-20 | rGrGrGrA------ | >60 | 58 |
| T4 | fUrGfCrGfUrAfCfUrGrGrGfCfUfCrAfUrAfUfUfCfCfCrGfCrGfCrAfUfC | | |
| Mut33 | | | |
| S2-20 | rGrGrGrA------fUrGfCrGrGrAfCfUrGrGrGfCfUfCrA-- | >60 | 59 |
| T4 | rAfUfUfCfCfCrGfCrGfCrAfUfC | | |
| Mut37 | | | |

SEQ ID NOs refer to the unmodified versions of the ligands described in the column titled, “Modified Sequence”; Sequences are listed in a 5′-3′ direction
rG = 2′Ribo G; rA = 2′Ribo A; fC = 2′-Fluoro C; fU = 2′-Fluoro U

The secondary structure of the truncated S2-20 sequence (FIG. 5) indicates there are two loop structures, where loop 1 is 5′-GAC-3′ and loop 2 is 5′-CUCAUAUU-3′. By conducting mutational analysis a more rigorous definition of the loop structures was able to be determined.

In loop 1 base 1, substitution to either A (mut 25), C (mut 7), or U (mut 33) was not well tolerated. Mut 7 had essentially no specific binding to CLEC-2 protein and a chemically synthesized version (RB588) was used as a negative control in further studies. The second base of loop 1 readily accepted substitution to either G (mut 31) or U (mut 8), suggesting this position is capable of tolerating either purines or pyrimidines. The double mutant (mut 1) binding data is consistent with the single point mutant data. The third base of loop 1 prefers to be a C. Substitution of the C to the other pyrimidine U (mut 26) was not well tolerated, nor was substitution to G (mut6), Given this data, loop1 can be described having the consensus sequence 5′-GNC-3′, where “N” represents any of the 4 bases (A, G, C, T or U).

Loop 2 is an eight base loop with the sequence 5′-CUCAUAUU-3′. Mutant analysis suggests a pyrimidine is preferred for base one. Substitution to a U (mut 17), was well tolerated, but a slight toss in affinity was observed. Substitution of base 1 to G (mut 9) was not tolerated well. A U is preferred at the second base of loop 2 as substitution to either an A (mut 10) or C (mut 18) was not tolerated well. A pyrimidine is preferred at the third base of loop 3 (mut 19), and substitution to a purine mut 11) was not well tolerated. Loop 2 base 4 could tolerate a purine (mut 20) or pyrimidine (mut 12) substitution. While this position can tolerate either a purine or a pyrimidine, deletion of the base was not tolerated (Mut 28). Base 5 could tolerate substitution to A (mut 13) or C (mut 21) with comparable binding affinity. In addition, the substitution to a G (mut 32) was also tolerated. While this position can readily tolerate substitutions, deleting this position is not tolerated (mut 37). A purine is preferred at loop 2 base 6. The purine substitution (mut22) was tolerated, but neither pyrimidine substitutions (mut 14 and mut 27) were tolerated. A pyrimidine is preferred at loop 2 base 7 (mut 23). Substitution to an A (mut 15) at base 7 results in an increase in Kd. While the binding of mutant 15 (Kd ˜30 nM) is significantly better than many of the other mutant constructs generated, a purine in this position is less desired than a pyrimidine. A U is preferred at loop 2 base 8. Substition of this U to either C (mut 24) or A (mut (6) were not well tolerated.

Based on this data, a preferred loop 2 consensus sequence can be written as follows:

5′-Y U Y N N R Y U-3′

where “Y” represents a pyrimidine, “R” represents a purine, and “N” represents any of the four bases.

### Example 5

### Degenerate SELEX

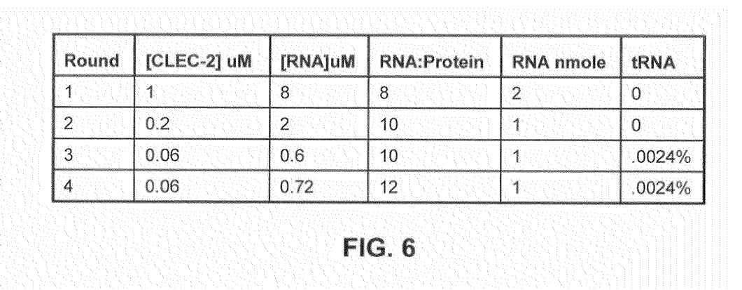

In order to identify key nucleotides within the S2-20 CLEC-2 ligand sequence, a degenerate SELEX was performed. The starting template oligo for the degenerate SELEX was the S2-20 sequence with degeneracy introduced by design into the N40 region. The synthesis of the S2-20 degenerate template oligo was 60% original base and 13.3% each of the remaining three bases throughout the S2-20 random region sequence. By introducing a degenerate version of the S2-20 sequence to the selective pressure of SELEX, one can determine which nucleotide substitutions are preferred for binding to CLEC-2. The S2-20 degenerate RNA starting pool had comparable binding as the naive Sel 2 pool for CLEC-2. After four rounds of selection, the binding affinity of the round 4 RNA pool from the degenerate selection was comparable to the round 10 RNA from the original selection. Specific conditions for the degenerate S2-20 selection are shown in FIG. 6. Forty-three aptamers from round 3 and forty-six aptamers from round 4 were cloned and sequenced as described in Example 2.

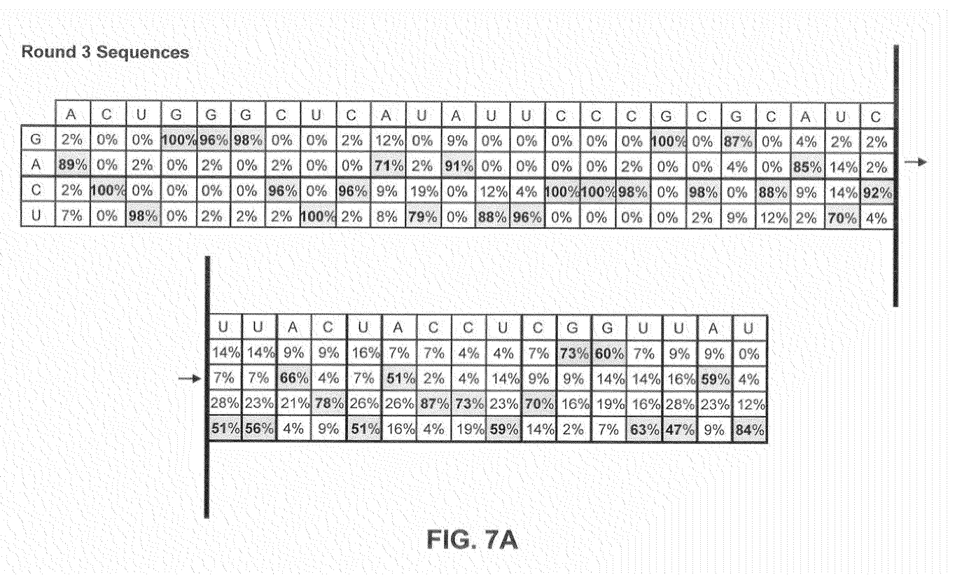

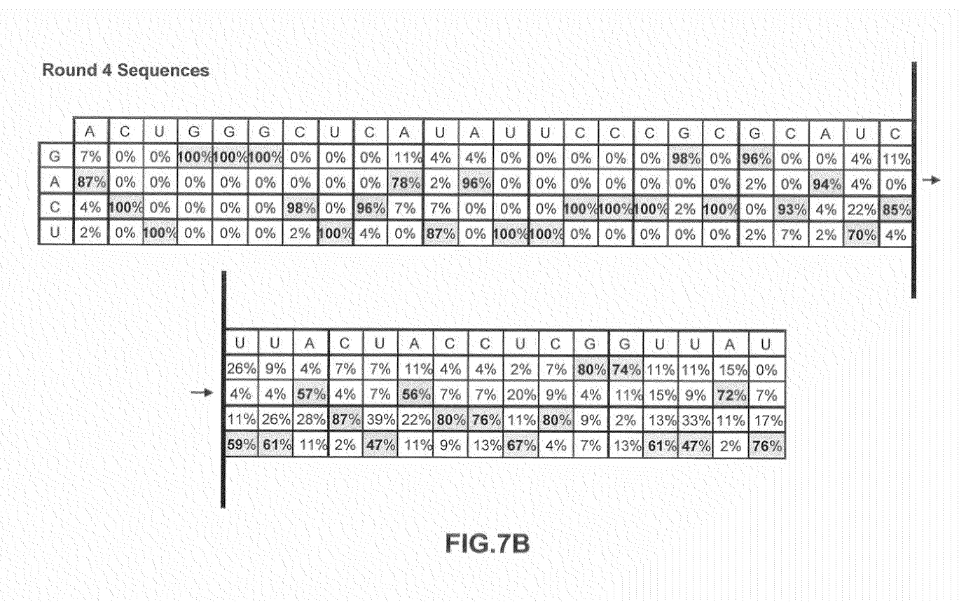

The starting degenerate pool was designed with 60% conservation of the initial sequence. By analyzing the substitution frequency rates at individual positions, one can determine which bases tolerate substitution readily. The frequency of each nucleotide at individual positions for both round 3 and round 4 sequences for the random region is illustrated in FIG. 7. The first 24 nucleotides in the random region of the sequences are highly conserved. Base positions 25-40 contain significantly less conservation, compared to the baseline of 60% as designed. These data are consistent with the truncation data, indicating the minimal essential region needed for binding to CLEC-2.

The degenerate selection analysis also revealed stem 1 could tolerate wobble base pairs as well as some minimal unpaired bases assuming the stem structure remains intact. Consistent with the truncation data, stem two was highly conserved in the degenerate selection, with the U-G base pair at the bottom of the stem being favored with a 100% frequency.

For loop 1, the first base (G) in the loop is derived from the 5′ fixed region and as a result no mutations were seen at that position. At loop 1 base 2 (A), there did appear to be a preference for A, but other substitutions were Observed and the representation of A at this position appeared to be decreasing as the SELEX progressed from round 3 to round 4. Consistent with the mutation data, the C at the third base in loop 1 was conserved in 100% of the sequences from both round 3 and round 4. For loop 2, similar observations were seen when comparing the degenerate selection results to the mutation data in Example 3.

Overall, the portion of the random region involved in binding to CLEC-2 (bases 1-24 of random region) converged highly to the original S2-20 sequence. The remaining bases of the random region (bases 25-40) which are not necessary in the minimal structure were much more divergent.

### Example 6

### Control Agents for Anti-CLEC-2 Ligand Sequence

Ligands encode the information necessary to design nucleic acid modulators, or control agents, for them based upon complementary Watson-Crick basepairing rules. The effixliveness of a given control agent is dependent upon several factors, including accessibility of the targeted region of the ligand for nucleation with the control agent, as well as the absence of or limited internal secondary structure within the control agent, which would require denaturation prior to full-duplex formation with the ligand. To define regions of the anti-CLEC-2 ligands that would be preferred regions for association with nucleic acid modulators, a series of control agents (See Table 4 and FIG. 8) were designed for RB587.

In order to test the effectiveness of all six control agents (RB581-586) a gel-shift assay was employed. In this assay, RB587 as end-labeled with γ-P³²-ATP and added at trace amounts to unlabeled RB587, and then incubated with either buffer or various molar ratios of one of the six control agents. More specifically, the molar concentration of unlabeled RB587 was kept constant at 1 μM while a range of control agent (8 μM to 0.25 μM) was added to the reaction resulting in a molar ratio range of 1:8 to 1:0.25 of RB587 to control agent. Reactions were incubated for 15 min at 37° C., loaded onto a 16% native polyacrylamide/0.5×TBE/2 mM CaCl₂ gel and run for 3 h at 10 W. The gel was then taken down, wrapped in Saran wrap, exposed to a phosphorimager screen for 1 h in a light-tight cassette and scanned by a Storm 840. The resulting scan showed the native position of RB587 alone, and RB587 plus varying molar amounts of control agent shifted upward due to hybridization of the ligand with the control agent. The results of the minimum molar ratio of control agent needed to disrupt native folding of RB587 are indicated in Table 4. The secondary structure of the anti-CLEC-2 ligand, RB587, is capable of being efficiently modulated by the use of control agents,

TABLE 4
Control Agents

| | | Min. | SEQ |
|---|---|---|---|
| Clone | | Molar | ID |
| Name | RNA Sequence | Ratio | NO: |
| RB581 | mGmAmUmGmCmGmCmGmGmGmAmAmUmAmUmG | 2:1 | 60 |
| RB582 | mUmGmCmGmCmGmGmGmAmAmUmAmUmGmAmG | 1:1 | 61 |
| RB583 | mAmAmUmAmUmGmAmGmCmCmCmAmGmUmC | 2:1 | 62 |
| RB584 | mUmAmUmGmAmGmCmCmCmAmGmUmCmCmGmC | 2:1 | 63 |
| RB585 | mAmUmGmAmGmCmCmCmAmGmUmCmCmGmCmAmUmC | 2:1 | 64 |
| RB586 | mCmCmCmAmGmUmCmCmGmCmAmUmC | 8:1 | 65 |

SEQ ID INOs refer to the unmodified versions of the modulators described in the column titled, “Modified Sequence”; Sequences are listed in a 5′-3′ direction
mU = 2′-O-methyl uridine; mA = 2′-O-methyl adenosine; 2′-O-methyl guanosine; mC = 2′-O-methyl cytosine

### Example 7

### Methods for Evaluating Antiplatelet Activity of Anti-CLEC-2 Ligands

### Washed Platelet Preparation (WP) and Aggregation Studies:

Human washed platelets were prepared essentially as described by Mustard et al. (1972; Br. J. Haematol 22, 193-204). Briefly, human blood was collected into one-sixth volume of acid/citrate/dextrose (ACID) buffer (85 mM sodium citrate, 65 mM citric acid, and 110 mM glucose), placed in a water bath at 37° C. for 30 min then centrifuged at 250×g for 16 min at room temperature. Platelet-rich plasma was removed and centrifuged at 2200×g for 13 min at room temperature then resuspended in 40 mL of HEPES-buffered Tyrode's solution (136.5 mM NaCl, 2.68 mM KCl, 1 mM MgCl₂, 2 mM CaCl₂, 12 mM NaHCO₃, 0.43 mM NaH₂PO₄, 5.5 mM glucose, 5 mM HEPES pH 7.4, 0.35% bovine serum albumin) containing 10 U/mL heparin and 5 μM (final concentration) prostaglandin I2 (PGI2). The platelet suspension was incubated in a 37° C. water bath for 10 min, 5 μM. (final concentration) PGI2 added and the mixture centrifuged at 1900×g for 8 min. The resulting pellet was resuspended in 40 mL of HEPES buffered Tyrode's solution containing 5 μM (final concentration) PGI2 and then incubated for 10 mM in a 37° C. water bath, and centrifuged at 1900×g for 8 min. The pellet is resuspended at a density of 3×10⁸ platelets/mL in HEPES-buffered Tyrode's solution containing 0.1 U/mL potato apyrase and incubated in a 37° C. water bath for 2 h prior to use in aggregometry studies.

Rhodocytin (Aggretin; purchased from Frankfurt University Hospital)-induced WP platelet aggregation was determined by measuring the transmission of light through a 0.5 ml suspension of stirred (1200 rpm) washed platelets (425 μl washed platelets, 25 μl fibrinogen, 25 μl of inhibitors or controls and 25 μl of rhodocytin) in a lumi-aggregometer at 37° C. (Chrono-Log Corp. Havertown, Pa.). The baseline of the instrument was set using 0.5 ml of Hepes-buffered Tyrode's solution. Prior to aggregation measurements, the platelet suspension was supplemented with 1 mg/ml fibrinogen final concentration. Platelet aggregation was initiated by the addition of indicated concentrations of rhodocytin (to yield a percent aggregation between 70-90%), and the light transmission was continuously recorded for at least 6 min. For screening anti-CLEC-2 ligands or controls for the ability to block Rhodocytin Induced Platelet Aggregation, anti-CLEC-2 ligands were added to the platelet suspension at a dilution to yield the desired final concentration, and incubated for 3 min before addition of rhodocytin, and the response was recorded for 4-6 min after rhodocytin addition.

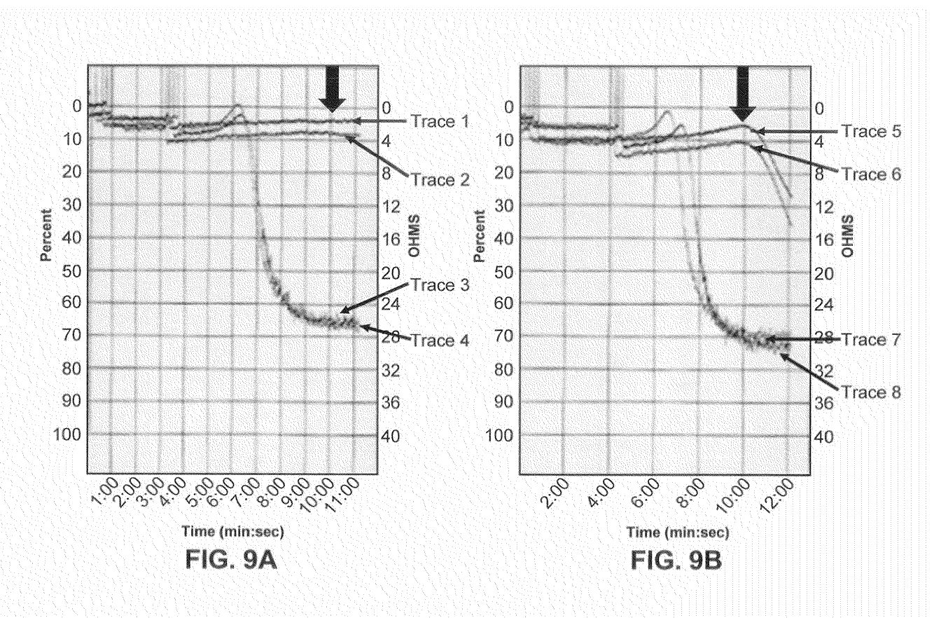

The potency of rhodocytin was determined for each donor from the maximal extent of percentage aggregation obtained from a dose response curve using serial dilutions of rhodocytin from approximately 3-60 nM range in 20 mM Tris pH 8.0, 50 mM. NaCl buffer, to determine an appropriate challenge concentration. The ability of anti-CLEC-2 ligands to inhibit Rhodocytin Induced Platelet Aggregation was tested in WP preparations as described above, using a broad range of anti-CLEC-2 ligand concentrations. FIGS. 9A and 9B shows graphs of rhodocytin-induced WP platelet aggregation of CLEC-2 ligands. S2-20 (Trace 2 and Trace 6; 1 μM and 0.5 μM); RB587 (Trace 1 and Trace 5; 1 μM and 0.5 μM); RB588 (Trace 3 and Trace 7; 1 μM and 0.5 μM); and Controls (Trace 4 and Trace 8; 20 nM rhodocytin). The position of the 6 minutes post rhodocytin addition is marked on the trace by thick downward pointing arrow. As shown in FIGS. 9A and 9B, the S2-20 and RB587 aptamers were fully effective in inhibiting rhodocytin-induced WP aggregation in the presence of approximately 20-30 nM rhodocytin agonist.

### Example 8

### Methods for Evaluating Antiplatelet Activity of Anti-CLEC-2 Ligands S2-20 Full Length Clone and RB587 Truncate in Rhodocytin-Induced Platelet Aggregation Assays

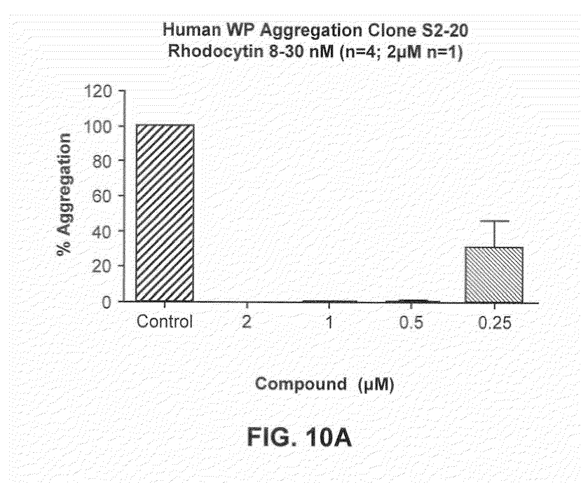

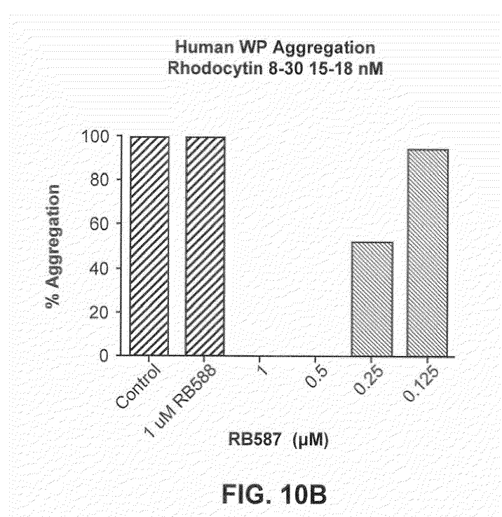

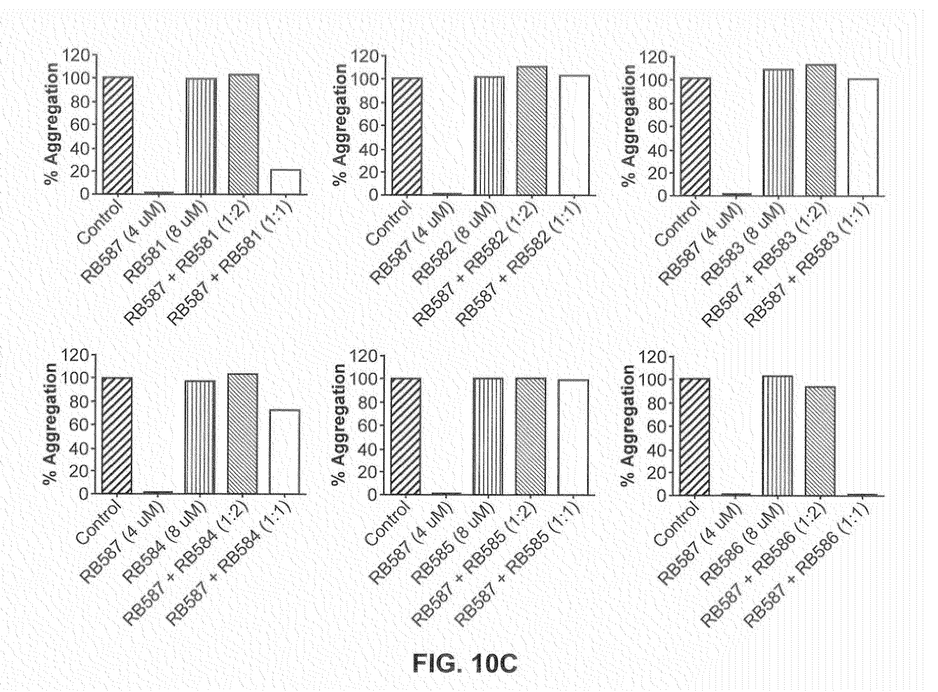

Human washed platelets were prepared essentially as described above. The potency of rhodocytin was determined for each donor from the maximal extent of percentage aggregation obtained from a dose response curve using serial dilutions of rhodocytin from approximately 3-60 nM, to determine an appropriate challenge concentration as described in Example 7. The ability of anti-CLEC-2 ligands to inhibit Rhodocytin Induced Platelet Aggregation was tested in WP preparation as described above. Data from the dose response analysis of S2-20 and RB587 is presented in FIGS. 10A and 10B. FIG. 10A is a graph of rhodocytin-induced platelet aggregation expressed as a percentage of control for varying concentrations of CLEC-2 nucleic acid ligand S2-20 clone in human washed platelets. FIG. 10B is a graph of rhodocytin-induced platelet aggregation expressed as a percentage of control for varying concentrations of CLEC-2 nucleic acid ligands RB587 and the inactive mutant RB588 in human washed platelets, Essentially full inhibition of aggregation in the presence of 0.5 μM. S2-20 or RB587 for 6 minutes after rhodocytin addition is observed.

### Rhodocytin-Induced Platelet Aggregation Assays for Nucleic Acid Modulators Testing of CLEC-2 Nucleic Acid Ligands WP:

Human Washed Platelets (WP) was prepared as described earlier. When a broad range of concentrations of the CLEC-2 nucleic acid ligand inhibitors were tested, an IC₉₅₋₁₀₀ value is obtained. The IC₉₅₋₁₀₀ values represent the concentration of inhibitor necessary to inhibit by about 95-100% the aggregation elicited by a given concentration of challenged agonists. When modulators are tested for their reversibility of anti-platelet activity, platelets are incubated for 3 min with 2-4 μM CLEC-2 aptamers (the IC₉₅₋₁₀₀) or buffer-F followed by the addition of different molar concentrations of modulators (4:1; 2:1; 1:1 modulators:aptamers) for 10 min before addition of rhodocytin, and the response is recorded for 6 minutes. FIG. 10C shows graphs of rhodocytin-induced platelet aggregation expressed as a percentage of control for varying concentrations of CLEC-2 nucleic acid ligand RB587 alone, or in combination with CLEC-2 ligand modulators RB581, RB582, RB583, RB584, RB585, and RB586 in human washed platelets.

### Example 9

### Platelet Activation Function Analysis Using Flow Cytometry (P-Selectin; CD62-P Expression) with Rhodocytin (Aggretin)

### Flow Cytometry Studies:

Flow cytometry, using a P-selectin-specific antibody, was performed to assess the ability of the CLEC-2 aptamers to inhibit rhodocytin-induced human P-selectin expression. P-selectin expression on the platelet surface is a marker for platelet activation. Human washed platelet suspension (density of 3×10⁸ cells/mL) was diluted to 1/10 in Tyrode's buffer and kept at 37° C., 5 μL of each test compound in buffer-F (SEL2 Pool, S2-20, S2-20 T4, S2-20 T5, S2-20 T6; RB587, and RB588 20 μM stock) was added in 5 mL assay tubes. Immediately, 50 μL of diluted platelets were added and mixed gently. The tubes were incubated at room temperature for 3 min. 2.5 μL of rhodocytin (3.331 μM stock was made in 20 mM Iris, 50 mM NaCl buffer, pH 8.0) was added to each tube, mixed and incubated for another 5 min. The tubes were transferred on ice and chilled for a minute. 20 μL of CD62P-PE antibody (Becton Dickinson; Cat #550561) was added to each tube and incubated for 20 min on ice. The platelet suspensions in each tube were further diluted with 0.6 mL of ice cold PBS and analyzed in FACS-Calibur.

### Detection CD62 Expression Using FACS-Calibur:

Two separate dot plots (one log SSC vs ESC and log FSC vs FL2-H) and a histogram were created on the flow viewing area. The platelets were identified using the dot plot of SSC vs ESC (the instrument was adjusted for proper viewing). A region of interest 1 (R1) around the platelets was created (this is to avoid debris counted as platelets). The platelets in region 1 (R1) was observed on the dot plot ISC vs FL2-H (adjusted FL2 using control). The platelets were also viewed on the histogram plotted counts vs FL2-H. A quadrant statistic marker was set on the FSC vs FL2-H dot blot using control tube and the statistical parameters were defined (control; unstained platelets). All the samples starting with resting control (plus antibody) were collected. The data is plotted as % of control (150 nM rhodocytin activation; lower right quadrant treated as the 100% activation total) using GraphPad Prism.

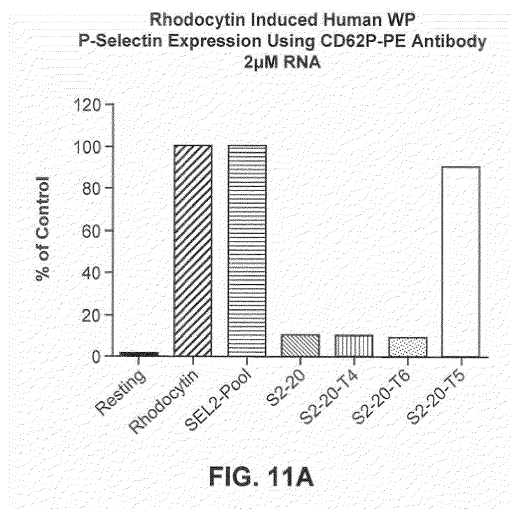

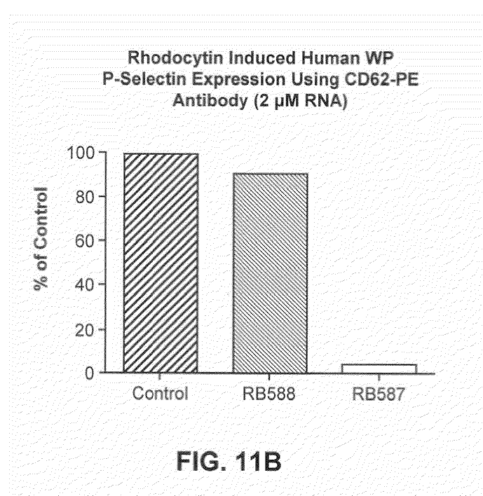

FACS analysis as described above was used to test the ability of the aptamers S2-20, RB587, and 3 truncates of S2-20, to inhibit rhodocytin-induced P-selectin expression in WP. The resultant data are illustrated in FIGS. 11A and 11B, which provide graphs of rhodocytin-induced human P-selectin expression using flow cytometry with P-selectin-PE antibody. The activity is expressed as a percentage of control for 2 μM of CLEC-2 nucleic acid ligand S2-20 clone and truncates in human washed platelets (FIG. 11A) or 2 μM of CLEC-2 nucleic acid ligand RB587 and the inactive mutant RB588 in human washed platelets (FIG. 11B).

CLEC-2 ligands S2-20, RB587, S2-20-T4, and S2-20-T6 were each able to significantly reduce rhodocytin-induced P-selectin expression on the surface of human WP, while truncates S2-20-T5 and RB588, which do not bind CLEC-2, showed no inhibitory activity, demonstrating the correlation between ligand affinity for CLEC-2 and inhibition of CLEC-2 activity.

### Example 10

### In Vitro Flow Based Platelet Adhesion Assay in Whole Blood for CLEC-2 Aptamer Activity Using Bioflux™ 200 (Fluxion Biosciences, Inc.)

CLEC-2 ligands were characterized for their ability to inhibit CLEC-2 mediated platelet aggregation using an in vitro flow-based platelet adhesion assay in the presence of whole blood.

### Whole Blood Preparation for Perfusion and Flow Experiment:

The blood was drawn from healthy volunteer into PPACK (0.3 mM) anticoagulant into 60 mL syringes using a 19 G×¾″ needle. The blood was immediately fluorescently labeled with 4 μM Calcein-AM (invitrogen P/N C3100 MP) for 1 hr at 37° C. (Calcein-AM was added to the blood very gently by inverting the tube a few times to mix and the blood was used within 3.5 h of draw). When the experiment was performed with fibrillar collagen or rhodocytin coated wells, the experiment was initiated by adding 200 μL of labeled blood to the inlet well, and perfusion was began immediately using 5 dyn/cm² whole blood flow settings at 37° C. using Bioflux™ software. When the experiment was performed with rat-tail collagen coated surface, the whole blood was activated with 165 nM rhodocytin for 2 minutes and then the experiment was initiated by adding 200 μL of labeled activated blood to the inlet well, and perfusion was began immediately using 5 dyn/cm² whole blood flow settings at 37° C. using Bioflux™ software. The data (fluorescence images of platelet aggregates) were collected using a time lapse fluorescence inverted microscope (Zeiss 200M Axiovert Microscope attached to an Axiocam Charged-Coupled Device camera and Axiovision software) for every 6 seconds for a total duration of 6-10 minutes. For testing the test articles (CLEC-2 aptamer RB587, and inactive ligand control RB588) a 200 μL, of labeled blood was incubated with indicated concentrations of aptamers (or buffer-F; in 10 μL volume) for 3-5 minutes at RT before addition to the inlet well. The tagged image file (tiff) formatted images were used to calculate fluorescence intensity using Bioflux Montage™ software and then the data table was exported to Microsoft excel and was plotted using Graphpad Prism (FIG. 12A and FIG. 12B).

Experiments with Rhodocytin Coated Surface:

Rhodocytin effectively stimulates CLEC-2 dependent platelet aggregation (Example 7; FIGS. 9 and 10). We analyzed platelet aggregate formation on a rhodocytin coated surface for studying CLEC-2 aptamers activity under flow conditions. For the flow experiments Bioflux 48 well plates (P/N 900-0017) were routinely used. For coating with rhodocytin, the plates were primed with 20 mM Tris pH 8.0, 50 in NI NaCl buffer for 5 min at 5 dyn/cm² and then 50 μg/ml of diluted rhodocytin was perfused from the outlet well for 10 min at 5 dyn/cm². Fibrillar collagen coated plates were used as controls. For collagen coating the plates were primed with 0.02 M acetic acid for 5 min at 5 dyn/cm² and then 25 μg/ml of diluted Fibrillar Collagen (Chrono-Log P/N 385) in 0.02 M acetic acid was perfused from outlet well for 10 min at 5 dyn/cm². The flow was stopped and the plate was incubated at room temp for 1 h. The coating proteins were washed with PBS at 5 dyn/cm² for 10 mM. The plate was blocked by completely filling the inlet well (1 ml) with 5% w/v BSA/PBS and was perfused into the channel at 5 dyn/cm² for 15 min. The flow was stopped and the plate was incubated for an additional 10 min at room temp. Excess PBS BSA was removed from all wells and the plate was kept at room temp for use the same day, or stored at 4° C. in PBS BSA for up to two days.

FIG. 12A shows video still fluorescent end-point images of the effects of CLEC-2 ligands on platelet accumulation on a rhodocytin-coated surface exposed to the flowing whole blood (A=50 μg/mL rhodocytin coating; B=25 μg/mL fibrillar collagen coating control; C through F coated with 50 μg/mL rhodocytin coating, C=3 μM RB587; D=3 μM RB588; E=4 μM RB588; and F=4 μM RB587). The graph G shows the measurement of adherent platelet surface coverage data calculated by Fluxion Montage™ software. The data is expressed as the percent maximum response of inactive mutant RB588 on the rhodocytin-coated surface. As shown in FIG. 12A, rhodocytin supported the accumulation of activated platelets in platelet aggregates under flow conditions in whole blood, RB587, but not the inactive mutant control RB588, specifically blocked this activity (FIG. 2A, compare panels C to D) and F to E).

Experiments with Soluble Rat-Tail Collagen-I Coated Surface:

We also analyzed the ability of the rhodocytin (CLEC-2 specific agonist) activated platelets to form larger platelet aggregates on rat-tail collagen coated surface compared to non-activated platelets under flow. We then analyzed the possible function of CLEC-2 in this process using RB587. For this assay, the plates were coated with 100 μg/mL soluble Rat-Tail Collagen (Invitrogen; Cat #A10483-01) as described above. The plates were primed with 0.02 acetic acid for 5 min at 5 dyn/cm² and then 25 μg/ml of either diluted Fibrilar Collagen (Chrono-Log P/N 385) or 100 μg/mL soluble Rat-Tail Collagen in 0.02 M acetic acid was perfused from outlet well for 10 min at 5 dyn/cm². The flow was stopped and the plate was incubated at room temp for 1 h. The coating proteins were washed with PBS at 5 dyn/cm² for 10 min. The plate was blocked by completely filling the inlet (1 ml) with 5% v/v BSA/PBS and was perfused into the channel 5 dyn/cm² for 15 min. The flow was stopped and the plate was incubated for an additional 10 minutes at room temp. Excess PBS BSA was removed from all wells and the plate was kept at room temp for use the same day, or store at 4° C. in PBS BSA for up to two days.

FIG. 12B shows video still fluorescent end-point images of the effects of CLEC-2 ligands on platelet accumulation on a rat-tail soluble collagen-coated surface exposed to the flowing whole blood in the presence and absence of rhodocytin agonist treatment (A=25 μg/mL fibrillar collagen; B=100 μg/mL rat-tail collagen; C=100 μg/mL rat-tail collagen plus 165 nM rhodocytin treatment; D=100 μg/mL rat-tail collagen plus buffer F; E=100 μg/mL rat-tail collagen plus 3 μM RB587 plus 165 nM rhodocytin treatment; F=100 μg/mL rat-tail collagen plus 3 μM RB588 plus 165 nM rhodocytin treatment). The graph G shows the measurement of adherent platelet coverage data calculated by Fluxion Montage™ software. The data is expressed as the percent maximum response of inactive mutant RB588 on a rat-tail collagen-coated surface in the presence of rhodocytin. As shown in FIG. 12B, the presence of the rhodocytin agonist significantly increased platelet aggregate size attaching to rat-tail collagen coated surface compared to untreated whole blood. When the whole blood was pre-incubated with RB587, followed by rhodocytin activation, the platelet aggregate size was reduced back to the baseline aggregates seen with soluble rat tail collagen in the absence of rhodocytin (FIG. 12B—images B, D, E, and the graph 6). The inactive mutant control, RB588, had no effect on platelet aggregate size (FIG. 12B panel F).

1. A nucleic acid ligand that binds CLEC-2, wherein said ligand comprises a nucleic acid sequence and wherein said nucleic acid sequence forms at least one stem structure and at least one loop structure, or a pharmaceutically acceptable salt thereof.

2. The ligand of claim 1, wherein the ligand comprises, in a 5′ to 3′ direction:
a first stem which is 5-10 basepairs in length;
a first trinucleotide loop which comprises the sequence 5′-GNC-3′;
a second stem which is 4 basepairs in length, wherein said second stem comprises a wobble pair at the base of the second stem; and
a second loop comprising the nucleotide sequence 5′-YUYNNRYU-3′.

3. The ligand claim 1, wherein said nucleic acid sequence comprises a sequence which is at least 80% identical to SEQ NO:7, SEQ ID NO:8 or SEQ ID NO:9.

4. The ligand of claim 1, wherein said nucleic acid sequence comprises a sequence which is at least 80% identical to SEQ ID NO:24.

5. The ligand of claim 1, wherein said ligand binds CLEC-2 with dissociation constant ranging from about 0.1 nM to 10 nM.

6. The nucleic acid ligand of claim 1, wherein said ligand comprises at least one modified nucleotide.

7. A modulator which binds specifically to a CLEC-2 ligand,
wherein said modulator comprises a second nucleic acid sequence; and
wherein said CLEC-2 ligand comprises a first nucleic acid sequence.

8. A method for treating a CLEC-2-mediated disorder comprising administering to a host in need thereof a therapeutically effective amount of a CLEC-2 ligand, or a pharmaceutically acceptable salt thereof.

9. The method of claim 8, wherein the CLEC-2-mediated disorder is a platelet-mediated disorder.

10. The method of claim 9, wherein the platelet-mediated disorder is selected from the group consisting of a vascular disorder, a cardiovascular disorder, a peripheral vascular disorder, a cerebrovascular disorder, a platelet-mediated inflammatory disorder, a diabetes-related disorder, a cancer, and HIV infection.

11. The method of claim 10, wherein the vascular disorder is selected from the group consisting of acute coronary syndromes, thrombosis, thromboembolism, thrombocytopenia, peripheral vascular disease, and transient ischemic attack.

12. The method of claim 10, wherein the cerebrovascular disorder is selected from the group consisting of transient ischemic attack, ischemic stroke, and embolism.

13. The method of claim 10, wherein the platelet-mediated inflammatory disorder selected from the group consisting of arthritis, rheumatoid arthritis, psoriatic arthritis, reactive arthritis, inflammatory bowed disease, ankylosing spondylitis, and scleroderma.

14. The method of claim 10, wherein the cancer is selected from the group consisting of lung cancer, breast cancer, prostate cancer, testicular cancer, pancreatic cancer, brain cancer, bone cancer and liver cancer.

15. The method of claim 10, wherein the diabetes-related disorder is selected from the group consisting of diabetic retinopathy, diabetic vasculopathy, atherosclerosis, ischemic stroke, peripheral vascular disease, acute renal injury and chronic renal failure.

16. A method for determining whether a CLEC-2 ligand activates or inhibits CLEC-2-dependent platelet aggregate formation, comprising
(a) mixing a CLEC-2 ligand with a blood sample to prepare a treated blood sample;
(b) contacting the treated blood sample with a facilitator molecule, wherein said facilitator molecule is immobilized on a solid support;
(c) measuring platelet aggregate formation after the contacting;
(d) comparing the degree of platelet aggregate formation detected in step (c) with the degree of platelet aggregation obtained when a control Hood sample with no CLEC-2 ligand is used to contact the facilitator molecule.

17. The method of claim 16, wherein the facilitator molecule is an activator of CLEC-2.

18. The method of claim 16, wherein the facilitator molecule is soluble collagen type I, II or III, and wherein the method further comprises adding rhodocytin to the blood sample.

19. The method of claim 16, further comprising mixing the treated blood sample with a modulator molecule prior to step (b), wherein said modulator molecule is capable of binding said regulating ligand molecule.

20. A kit comprising a CLEC-2 ligand and a pharmaceutical excipient.

21. The kit according to claim 20, further comprising a modulator which specifically binds the CLEC-2 ligand.

22. A kit comprising a modulator which specifically binds to a CLEC-2 ligand.
